SEQUENCE LISTING (130 sequences)

SEQ ID NO: 1 - DNA, 58 bp, Artificial sequence
  Single strand DNA oligonucleotide
ttctaatacgactcactataggggctatagctcagctgggagagcgcttgcatggcat

SEQ ID NO: 2 - DNA, 58 bp, Artificial sequence
  Single strand DNA oligonucleotide
tggtggagctaagcgggatcgaaccgctgacctcttgcatgccatgcaagcgctctcc

SEQ ID NO: 3 - DNA, 59 bp, Artificial sequence
  Single strand DNA oligonucleotide
ttctaatacgactcactatagcgcccgtagctcagctggatagagcgctgccctccgga

SEQ ID NO: 4 - DNA, 59 bp, Artificial sequence
  Single strand DNA oligonucleotide
tggcgcgcccgacaggattcgaacctgagacctctgcctccggagggcagcgctctatc

SEQ ID NO: 5 - DNA, 57 bp, Artificial sequence
  Single strand DNA oligonucleotide
ttctaatacgactcactataggaggactgatgagtcggaaacgacgaaacgcgaaag

SEQ ID NO: 6 - DNA, 55 bp, Artificial sequence
  Single strand DNA oligonucleotide
ccgccgttctaccgactgaactacagaggagacgctttcgcgtttcgtcgtttcc

SEQ ID NO: 7 - DNA, 47 bp, Artificial sequence
  Single strand DNA oligonucleotide
cggtagaacggcggactgttaatccgtatgtcactggttcgagtcca

SEQ ID NO: 8 - DNA, 34 bp, Artificial sequence
  Single strand DNA oligonucleotide
tggctcctctgactggactcgaaccagtgacata

SEQ ID NO: 9 - DNA, 60 bp, Artificial sequence
  Single strand DNA oligonucleotide
ttctaatacgactcactataggagcggtagttcagtcggttagaatacctgcctgtcacg

SEQ ID NO: 10 - DNA, 60 bp, Artificial sequence
  Single strand DNA oligonucleotide
tggcggaacggacgggactcgaacccgcgaccccctgcgtgacaggcaggtattctaacc

SEQ ID NO: 11 - DNA, 57 bp, Artificial sequence
  Single strand DNA oligonucleotide
ttctaatacgactcactataggcgcgttaacaaagcggttatgtagcggattgcaaa

SEQ ID NO: 12 - DNA, 57 bp, Artificial sequence
  Single strand DNA oligonucleotide
tggaggcgcgttccggagtcgaaccggactagacggatttgcaatccgctacataac

SEQ ID NO: 13 - DNA, 59 bp, Artificial sequence
  Single strand DNA oligonucleotide
ttctaatacgactcactataggcggggtggagcagcctggtagctcgtcgggctcataa

SEQ ID NO: 14 - DNA, 59 bp, Artificial sequence
  Single strand DNA oligonucleotide
tggttgcgggggccggatttgaaccgacgatcttcgggttatgagcccgacgagctacc

SEQ ID NO: 15 - DNA, 33 bp, Artificial sequence
  Single strand DNA oligonucleotide
ttctaatacgactcactataggccccactgatg

SEQ ID NO: 16 - DNA, 50 bp, Artificial sequence
  Single strand DNA oligonucleotide
cagacgctttcgcgtttcgtcgtttccgactcatcagtggggcctatagt

SEQ ID NO: 17 - DNA, 59 bp, Artificial sequence
  Single strand DNA oligonucleotide
acgcgaaagcgtctggggtatcgccaagcggtaaggcaccggattctgattccggcatt

SEQ ID NO: 18 - DNA, 57 bp, Artificial sequence
  Single strand DNA oligonucleotide
tggctggggtacgaggattcgaacctcggaatgccggaatcagaatccggtgcctta

SEQ ID NO: 19 - DNA, 39 bp, Artificial sequence
  Single strand DNA oligonucleotide
taatacgactcactatagggacctgatgagtcggaaacg

SEQ ID NO: 20 - DNA, 60 bp, Artificial sequence
  Single strand DNA oligonucleotide
gacctgatgagtcggaaacgacgaaacgcgaaagcgtcgtccccttcgtctagaggccca

SEQ ID NO: 21 - DNA, 57 bp, Artificial sequence
  Single strand DNA oligonucleotide
attcgaacccctgttaccgccgtgagagggcggtgtcctgggcctctagacgaaggg

SEQ ID NO: 22 - DNA, 35 bp, Artificial sequence
  Single strand DNA oligonucleotide
tggcgtcccctaggggattcgaacccctgttaccg

SEQ ID NO: 23 - DNA, 58 bp, Artificial sequence
  Single strand DNA oligonucleotide
ttctaatacgactcactatagcgggaatagctcagttggtagagcacgaccttgccaa

SEQ ID NO: 24 - DNA, 58 bp, Artificial sequence
  Single strand DNA oligonucleotide
tggagcgggaaacgagactcgaactcgcgaccccgaccttggcaaggtcgtgctctac

SEQ ID NO: 25 - DNA, 58 bp, Artificial sequence
  Single strand DNA oligonucleotide
ttctaatacgactcactatagtggctatagctcagttggtagagccctggattgtgat

SEQ ID NO: 26 - DNA, 58 bp, Artificial sequence
  Single strand DNA oligonucleotide
tggggtggctaatgggattcgaacccacgacaactggaatcacaatccagggctctac

SEQ ID NO: 27 - DNA, 59 bp, Artificial sequence
  Single strand DNA oligonucleotide
ttctaatacgactcactatagggcttgtagctcaggtggttagagcgcacccctgataa

SEQ ID NO: 28 - DNA, 59 bp, Artificial sequence
  Single strand DNA oligonucleotide
tggtgggcctgagtggacttgaaccaccgacctcacccttatcaggggtgcgctctaac

SEQ ID NO: 29 - DNA, 36 bp, Artificial sequence
  Single strand DNA oligonucleotide
ttctaatacgactcactatagccgaggtggtggaat

SEQ ID NO: 30 - DNA, 39 bp, Artificial sequence
  Single strand DNA oligonucleotide
acctcaaggtagcgtgtctaccaattccaccacctcggc

SEQ ID NO: 31 - DNA, 44 bp, Artificial sequence
  Single strand DNA oligonucleotide
acacgctaccttgaggtggtagtgcccaatagggcttacgggtt

SEQ ID NO: 32 - DNA, 43 bp, Artificial sequence
  Single strand DNA oligonucleotide
tggtaccgaggacgggacttgaacccgtaagccctattgggca

SEQ ID NO: 33 - DNA, 58 bp, Artificial sequence
  Single strand DNA oligonucleotide
ttctaatacgactcactatagggtcgttagctcagttggtagagcagttgactcttaa

SEQ ID NO: 34 - DNA, 58 bp, Artificial sequence
  Single strand DNA oligonucleotide
tggtgggtcgtgcaggattcgaacctgcgaccaattgattaagagtcaactgctctac

SEQ ID NO: 35 - DNA, 58 bp, Artificial sequence
  Single strand DNA oligonucleotide
ttctaatacgactcactataggctacgtagctcagttggttagagcacatcactcata

SEQ ID NO: 36 - DNA, 58 bp, Artificial sequence
  Single strand DNA oligonucleotide
tggtggctacgacgggattcgaacctgtgaccccatcattatgagtgatgtgctctaa

SEQ ID NO: 37 - DNA, 58 bp, Artificial sequence
  Single strand DNA oligonucleotide
ttctaatacgactcactatagcccggatagctcagtcggtagagcaggggattgaaaa

SEQ ID NO: 38 - DNA, 58 bp, Artificial sequence
  Single strand DNA oligonucleotide
tggtgcccggactcggaatcgaaccaaggacacggggattttcaatcccctgctctac

SEQ ID NO: 39 - DNA, 55 bp, Artificial sequence
  Single strand DNA oligonucleotide
ttctaatacgactcactataggcaccgctgatgagtcggaaacgacgaaacgcga

SEQ ID NO: 40 - DNA, 52 bp, Artificial sequence
  Single strand DNA oligonucleotide
ccaggctgcgccaatcaccggacgctttcgcgtttcgtcgtttccgactcat

SEQ ID NO: 41 - DNA, 53 bp, Artificial sequence
  Single strand DNA oligonucleotide
ggcgcagcctggtagcgcacttcgttcgggacgaaggggtcggaggttcgaat

SEQ ID NO: 42 - DNA, 36 bp, Artificial sequence
  Single strand DNA oligonucleotide
tggtcggtgatagaggattcgaacctccgacccctt

SEQ ID NO: 43 - DNA, 41 bp, Artificial sequence
  Single strand DNA oligonucleotide
ttctaatacgactcactataggagagatgccggagcggctg

SEQ ID NO: 44 - DNA, 47 bp, Artificial sequence
  Single strand DNA oligonucleotide
gcccctactccggttttcgagaccggtccgttcagccgctccggcat

SEQ ID NO: 45 - DNA, 51 bp, Artificial sequence
  Single strand DNA oligonucleotide
accggagtaggggcaactctaccgggggttcaaatccccctctctccgcca

SEQ ID NO: 46 - DNA, 19 bp, Artificial sequence
  Single strand DNA oligonucleotide
tggcggagagagggggatt

SEQ ID NO: 47 - DNA, 58 bp, Artificial sequence
  Single strand DNA oligonucleotide
ttctaatacgactcactatagccgatatagctcagttggtagagcagcgcattcgtaa

SEQ ID NO: 48 - DNA, 58 bp, Artificial sequence
  Single strand DNA oligonucleotide
tggtgccgataataggagtcgaacctacgaccttcgcattacgaatgcgctgctctac

SEQ ID NO: 49 - DNA, 32 bp, Artificial sequence
  Single strand DNA oligonucleotide
ttctaatacgactcactataggcccctctgat

SEQ ID NO: 50 - DNA, 57 bp, Artificial sequence
  Single strand DNA oligonucleotide
cgcccctgacgctttcgcgtttcgtcgtttccgactcatcagaggggcctatagtga

SEQ ID NO: 51 - DNA, 52 bp, Artificial sequence
  Single strand DNA oligonucleotide
gcgtcaggggcgtagttcaattggtagagcaccggtctccaaaaccgggtgt

SEQ ID NO: 52 - DNA, 48 bp, Artificial sequence
  Single strand DNA oligonucleotide
tggcaggggcggagagactcgaactcccaacacccggttttggagacc

SEQ ID NO: 53 - DNA, 42 bp, Artificial sequence
  Single strand DNA oligonucleotide
ttctaatacgactcactataggtggggttcccgagcggccaa

SEQ ID NO: 54 - DNA, 37 bp, Artificial sequence
  Single strand DNA oligonucleotide
cggcagatttacagtctgctccctttggccgctcggg

SEQ ID NO: 55 - DNA, 42 bp, Artificial sequence
  Single strand DNA oligonucleotide
gcagactgtaaatctgccgtcatcgacttcgaaggttcgaat

SEQ ID NO: 56 - DNA, 38 bp, Artificial sequence
  Single strand DNA oligonucleotide
tggtggtgggggaaggattcgaaccttcgaagtcgatg

SEQ ID NO: 57 - DNA, 59 bp, Artificial sequence
  Single strand DNA oligonucleotide
ttctaatacgactcactatagcgtccgtagctcagttggttagagcaccaccttgacat

SEQ ID NO: 58 - DNA, 59 bp, Artificial sequence
  Single strand DNA oligonucleotide
tggtgcgtccgagtggactcgaaccaccgacccccaccatgtcaaggtggtgctctaac

SEQ ID NO: 59 - DNA, 43 bp, Artificial sequence
  Single strand DNA oligonucleotide
ttctaatacgactcactataggatcgaaagatttccgcatccc

SEQ ID NO: 60 - DNA, 43 bp, Artificial sequence
  Single strand DNA oligonucleotide
acctaacgccatgtaccctttcggggatgcggaaatctttcga

SEQ ID NO: 61 - DNA, 43 bp, Artificial sequence
  Single strand DNA oligonucleotide
ttctaatacgactcactataggatcgaaagatttccgcggccc

SEQ ID NO: 62 - DNA, 38 bp, Artificial sequence
  Single strand DNA oligonucleotide
acctaacgctaatcccctttcggggccgcggaaatctt

SEQ ID NO: 63 - DNA, 49 bp, Artificial sequence
  Single strand DNA oligonucleotide
ggcgtaatacgactcactatagggttaactttaagaaggagatatacca

SEQ ID NO: 64 - DNA, 40 bp, Artificial sequence
  Single strand DNA oligonucleotide
ttaactttaagaaggagatataccaatgaagtacgacaag

SEQ ID NO: 65 - DNA, 38 bp, Artificial sequence
  Single strand DNA oligonucleotide
cgaagctcagtcgtacttgtcgtacttcattggtatat

SEQ ID NO: 66 - DNA, 41 bp, Artificial sequence
  Single strand DNA oligonucleotide
ttaactttaagaaggagatataccaatgaagaagtacgact

SEQ ID NO: 67 - DNA, 43 bp, Artificial sequence
  Single strand DNA oligonucleotide
cgaagcttacatccgcgagtcgtacttcttcattggtatatct

SEQ ID NO: 68 - DNA, 42 bp, Artificial sequence
  Single strand DNA oligonucleotide
ttaactttaagaaggagatataccaatgaagtggctcccgaa

SEQ ID NO: 69 - DNA, 43 bp, Artificial sequence
  Single strand DNA oligonucleotide
cgaagcttaggccgtctgcttcgggagccacttcattggtata

SEQ ID NO: 70 - DNA, 44 bp, Artificial sequence
  Single strand DNA oligonucleotide
ttaactttaagaaggagatataccaatgttcgagtgccacaacg

SEQ ID NO: 71 - DNA, 43 bp, Artificial sequence
  Single strand DNA oligonucleotide
cgaagcttacttgccgatcttgacgttgtggcactcgaacatt

SEQ ID NO: 72 - DNA, 45 bp, Artificial sequence
  Single strand DNA oligonucleotide
ttaactttaagaaggagatataccaatgaaggtcaagtggcagcc

SEQ ID NO: 73 - DNA, 45 bp, Artificial sequence
  Single strand DNA oligonucleotide
cgaagcttacttgagcggctgcggctgcggctgccacttgacctt

SEQ ID NO: 74 - DNA, 56 bp, Artificial sequence
  Single strand DNA oligonucleotide
ttaactttaagaaggagatataccaatgatcacgcggaacgcctgccacgcccggg

SEQ ID NO: 75 - DNA, 53 bp, Artificial sequence
  Single strand DNA oligonucleotide
cgaagcttacatctccgtcgagtacgagccgttgatgccccgggcgtggcagg

SEQ ID NO: 76 - DNA, 99 bp, Artificial sequence
  L-nucleic acid oligo sequence
tggtggagctaagcgggatcgaaccgctgacctcttgcatgccatgcaagcgctctcccagctgagctatagcccctatagtgagtcgtattagaaccg

SEQ ID NO: 77 - DNA, 99 bp, Artificial sequence
  L-nucleic acid oligo sequence
tggagcgggaaacgagactcgaactcgcgaccccgaccttggcaaggtcgtgctctaccaactgagctattcccgctatagtgagtcgtattagaaccg

SEQ ID NO: 78 - DNA, 99 bp, Artificial sequence
  L-nucleic acid oligo sequence
tggtgggtcgtgcaggattcgaacctgcgaccaattgattaagagtcaactgctctaccaactgagctaacgaccctatagtgagtcgtattagaaccg

SEQ ID NO: 79 - DNA, 99 bp, Artificial sequence
  L-nucleic acid oligo sequence
tggtgcccggactcggaatcgaaccaaggacacggggattttcaatcccctgctctaccgactgagctatccgggctatagtgagtcgtattagaaccg

SEQ ID NO: 80 - DNA, 24 bp, Artificial sequence
  nucleic acid oligo sequence
cggttctaatacgactcactatag

SEQ ID NO: 81 - DNA, 23 bp, Artificial sequence
  nucleic acid oligo sequence
cggttctaatacgactcactata

SEQ ID NO: 82 - RNA, 46 nt, Artificial sequence
  L-flexizyme nucleic acid sequence
ggaucgaaagauuuccgcauccccgaaaggguacauggcguuaggu

SEQ ID NO: 83 - DNA, 393 bp, Artificial sequence
  Chicken lysozyme
atgaaggtcttcggccggtgcgagctcgccgccgccatgaagcggcacggcctcgacaactaccggggctactcgctcggcaactgggtctgcgccgccaagttcgagtcgaacttcaacacgcaggccacgaaccggaacacggacggctcgacggactacggcatcctccagatcaactcgcggtggtggtgcaacgacggccggacgccgggctcgcggaacctctgcaacatcccgtgctcggccctcctctcgtcggacatcacggcctcggtcaactgcgccaagaagatcgtctcggacggcaacggcatgaacgcctgggtcgcctggcggaaccggtgcaagggcacggacgtccaggcctggatccggggctgccggctctaa

SEQ ID NO: 84 - DNA, 510 bp, Artificial sequence
  Gaussia luciferase
atgaagccgacggagaacaacgaggacttcaacatcgtcgccgtcgcctcgaacttcgccacgacggacctcgacgccgaccggggcaagctcccgggcaagaagctcccgctcgaggtcctcaaggagatggaggccaacgcccggaaggccggctgcacgcggggctgcctcatctgcctctcgcacatcaagtgcacgccgaagatgaagaagttcatcccgggccggtgccacacgtacgagggcgacaaggagtcggcccagggcggcatcggcgaggccatcgtcgacatcccggagatcccgggcttcaaggacctcgagccgatggagcagttcatcgcccaggtcgacctctgcgtcgactgcacgacgggctgcctcaagggcctcgccaacgtccagtgctcggacctcctcaagaagtggctcccgcagcggtgcgccacgttcgcctcgaagatccagggccaggtcgacaagatcaagggcgccggcggcgactaa

SEQ ID NO: 85 - DNA, 1005 bp, Escherichia coli
atgacgaagccgatcgtcttctcgggcgcccagccgtcgggcgagctcacgatcggcaactacatgggcgccctccggcagtgggtcaacatgcaggacgactaccactgcatctactgcatcgtcgaccagcacgccatcacggtccggcaggacgcccagaagctccggaaggccacgctcgacacgctcgccctctacctcgcctgcggcatcgacccggagaagtcgacgatcttcgtccagtcgcacgtcccggagcacgcccagctcggctgggccctcaactgctacacgtacttcggcgagctctcgcggatgacgcagttcaaggacaagtcggcccggtacgccgagaacatcaacgccggcctcttcgactacccggtcctcatggccgccgacatcctcctctaccagacgaacctcgtcccggtcggcgaggaccagaagcagcacctcgagctctcgcgggacatcgcccagcggttcaacgccctctacggcgagatcttcaaggtcccggagccgttcatcccgaagtcgggcgcccgggtcatgtcgctcctcgagccgacgaagaagatgtcgaagtcggacgacaaccggaacaacgtcatcggcctcctcgaggacccgaagtcggtcgtcaagaagatcaagcgggccgtcacggactcggacgagccgccggtcgtccggtacgacgtccagaacaaggccggcgtctcgaacctcctcgacatcctctcggccgtcacgggccagtcgatcccggagctcgagaagcagttcgagggcaagatgtacggccacctcaagggcgaggtcgccgacgccgtctcgggcatgctcacggagctccaggagcggtaccaccggttccggaacgacgaggccttcctccagcaggtcatgaaggacggcgccgagaaggcctcggtccacgcctcgcggacgctcaaggccgtctacgaggccatcggcttcgtcgccaagccgtaa

SEQ ID NO: 86 - RNA, 35 nt, Artificial sequence
  Single strand oligonucleotide
aggggcguaguucaauugguagagcaccggucucc

SEQ ID NO: 87 - DNA, 41 bp, Artificial sequence
  Single strand oligonucleotide
  misc_feature (1)..(1): 5′ phosphorylated
  misc_feature (11)..(11): conjugated to Cy5
aaaaccgggutuugggaguucgagucucuccgccccugcca

SEQ ID NO: 88 - RNA, 76 nt, Artificial sequence
  tRNA sequence
ggggcuauagcucagcugggagagcgcuugcauggcaugcaagaggucagcgguucgaucccgcuuagcuccacca

SEQ ID NO: 89 - RNA, 77 nt, Artificial sequence
  tRNA sequence
gcgcccguagcucagcuggauagagcgcugcccuccggaggcagaggucucagguucgaauccugucgggcgcgcca

SEQ ID NO: 90 - RNA, 76 nt, Artificial sequence
  tRNA sequence
uccucuguaguucagucgguagaacggcggacuguuaauccguaugucacugguucgaguccagucagaggagcca

SEQ ID NO: 91 - RNA, 77 nt, Artificial sequence
  tRNA sequence
ggagcgguaguucagucgguuagaauaccugccugucacgcagggggucgcggguucgagucccguccguuccgcca

SEQ ID NO: 92 - RNA, 74 nt, Artificial sequence
  tRNA sequence
ggcgcguuaacaaagcgguuauguagcggauugcaaauccgucuaguccgguucgacuccggaacgcgccucca

SEQ ID NO: 93 - RNA, 77 nt, Artificial sequence
  tRNA sequence
ggcgggguggagcagccugguagcucgucgggcucauaacccgaagaucgucgguucaaauccggcccccgcaacca

SEQ ID NO: 94 - RNA, 75 nt, Artificial sequence
  tRNA sequence
ugggguaucgccaagcgguaaggcaccggauucugauuccggcauuccgagguucgaauccucguaccccagcca

SEQ ID NO: 95 - RNA, 75 nt, Artificial sequence
  tRNA sequence
gccccuucgucuagaggcccaggacaccgcccucucacggcgguaacagggguucgaauccccuaggggacgcca

SEQ ID NO: 96 - RNA, 76 nt, Artificial sequence
  tRNA sequence
gcgggaauagcucaguugguagagcacgaccuugccaaggucggggucgcgaguucgagucucguuucccgcucca

SEQ ID NO: 97 - RNA, 76 nt, Artificial sequence
  tRNA sequence
guggcuauagcucaguugguagagcccuggauugugauuccaguugucguggguucgaaucccauuagccacccca

SEQ ID NO: 98 - RNA, 77 nt, Artificial sequence
  tRNA sequence
gggcuuguagcucaggugguuagagcgcaccccugauaagggugaggucggugguucaaguccacucaggcccacca

SEQ ID NO: 99 - RNA, 87 nt, Artificial sequence
  tRNA sequence
gccgaggugguggaauugguagacacgcuaccuugaggugguagugcccaauagggcuuacggguucaagucccguccucgguacca

SEQ ID NO: 100 - RNA, 76 nt, Artificial sequence
  tRNA sequence
gggucguuagcucaguugguagagcaguugacucuuaaucaauuggucgcagguucgaauccugcacgacccacca

SEQ ID NO: 101 - RNA, 77 nt, Artificial sequence
  tRNA sequence
ggcuacguagcucaguugguuagagcacaucacucauaaugauggggucacagguucgaaucccgucguagccacca

SEQ ID NO: 102 - RNA, 76 nt, Artificial sequence
  tRNA sequence
gcccggauagcucagucgguagagcaggggauugaaaauccccguguccuugguucgauuccgaguccgggcacca

SEQ ID NO: 103 - RNA, 77 nt, Artificial sequence
  tRNA sequence
cggugauuggcgcagccugguagcgcacuucguucgggacgaaggggucggagguucgaauccucuaucaccgacca

SEQ ID NO: 104 - RNA, 90 nt, Artificial sequence
  tRNA sequence
ggagagaugccggagcggcugaacggaccggucucgaaaaccggaguaggggcaacucuaccggggguucaaaucccccucucuccgcca

SEQ ID NO: 105 - RNA, 76 nt, Artificial sequence
  tRNA sequence
gccgauauagcucaguugguagagcagcgcauucguaaugcgaaggucguagguucgacuccuauuaucggcacca

SEQ ID NO: 106 - RNA, 76 nt, Artificial sequence
  tRNA sequence
aggggcguaguucaauugguagagcaccggucuccaaaaccggguguugggaguucgagucucuccgccccugcca

SEQ ID NO: 107 - RNA, 85 nt, Artificial sequence
  tRNA sequence
ggugggguucccgagcggccaaagggagcagacuguaaaucugccgucaucgacuucgaagguucgaauccuucccccaccacca

SEQ ID NO: 108 - RNA, 77 nt, Artificial sequence
  tRNA sequence
gcguccguagcucaguugguuagagcaccaccuugacauggugggggucggugguucgaguccacucggacgcacca

SEQ ID NO: 109 - protein, 19 aa, Artificial sequence
  Detected peptide sequence of aaRS-free translated E. coli TrpRS by LC-MS/MS
  MISC_FEATURE (13)..(13): Cysteine Carbamidomethylation (CAM)
KATLDTLALYLAXGIDPEK

SEQ ID NO: 110 - protein, 12 aa, Artificial sequence
  Detected peptide sequence of aaRS-free translated E. coli TrpRS by LC-MS/MS
AVYEAIGFVAKP

SEQ ID NO: 111 - protein, 12 aa, Artificial sequence
  Detected peptide sequence of aaRS-free translated E. coli TrpRS by LC-MS/MS
AVTDSDEPPVVR

SEQ ID NO: 112 - protein, 10 aa, Artificial sequence
  Detected peptide sequence of aaRS-free translated E. coli TrpRS by LC-MS/MS
FNALYGEIFK

SEQ ID NO: 113 - RNA, 77 nt, Artificial sequence; identical to SEQ ID NO: 91
  tRNA sequence

SEQ ID NO: 114 - RNA, 77 nt, Artificial sequence; identical to SEQ ID NO: 93
  tRNA sequence

SEQ ID NO: 115 - RNA, 76 nt, Artificial sequence
  tRNA sequence
guccccuucgucuagaggcccaggacaccgcccucuaacggcgguaacagggguucgaauccccuaggggacgcca

SEQ ID NO: 116 - RNA, 76 nt, Artificial sequence; identical to SEQ ID NO: 100
  tRNA sequence

SEQ ID NO: 117 - RNA, 76 nt, Artificial sequence; identical to SEQ ID NO: 102
  tRNA sequence

SEQ ID NO: 118 - RNA, 85 nt, Artificial sequence; identical to SEQ ID NO: 107
  tRNA sequence

SEQ ID NO: 119 - DNA, 103 bp, Artificial sequence
  DNA template sequences for in vitro translation
ggcgtaatacgactcactatagggttaactttaagaaggagatataccaatgaagaagaagttcttcttcgactacaaggacgacgacgacaagtaagcttcg

SEQ ID NO: 120 - DNA, 100 bp, Artificial sequence
  DNA template sequences for in vitro translation
ggcgtaatacgactcactatagggttaactttaagaaggagatataccaatgaagaagaagtagtaggactacaaggacgacgacgacaagtaagcttcg

SEQ ID NO: 121 - DNA, 103 bp, Artificial sequence
  DNA template sequences for in vitro translation
ggcgtaatacgactcactatagggttaactttaagaaggagatataccaatgaagaagaagtagtagtaggactacaaggacgacgacgacaagtaagcttcg

SEQ ID NO: 122 - DNA, 103 bp, Artificial sequence; identical to SEQ ID NO: 121
  DNA template sequences for in vitro translation

SEQ ID NO: 123 - protein, 20 aa, Artificial sequence
  short message peptide encoded into mRNA #6
MITRNACHARGINGSYSTEM

SEQ ID NO: 124 - protein, 18 aa, Artificial sequence
  peptide drug previously shown to inhibit human factor XIIa
  MISC_FEATURE (1)..(1): acetyl-D-Tyr
  MISC_FEATURE (8)..(8): 2-aminocyclohexanecarboxylic acid (2-ACHC)
  MISC_FEATURE (13)..(13): 2-aminocyclohexanecarboxylic acid (2-ACHC)
  MISC_FEATURE (17)..(17): D-Cys
  MOD_RES (18)..(18): C′ amidated
YFAYDRRXLSNNXRNYCG

SEQ ID NO: 125 - protein, 7 aa, Artificial sequence
  heptapeptides
  MISC_FEATURE (1)..(1): fluorescein (FAM)-labeled
FKYDKYD

SEQ ID NO: 126 - protein, 352 aa, Artificial sequence
  L-Dpo4-5m-Y12S, (Y12S, C31S, S86C, N123A, S207A, S313A)
MIVLFVDFDYFSAQVEEVLNPSLKGKPVVVSVFSGRFEDSGAVATANYEARKFGVKAGIPIVEAKKILPNAVYLPMRKEVYQQVSCRIMNLLREYSEKIEIASIDEAYLDISDKVRDYREAYALGLEIKNKILEKEKITVTVGISKNKVFAKIAADMAKPNGIKVIDDEEVKRLIRELDIADVPGIGNITAEKLKKLGINKLVDTLAIEFDKLKGMIGEAKAKYLISLARDEYNEPIRTRVRKSIGRIVTMKRNSRNLEEIKPYLFRAIEESYYKLDKRIPKAIHVVAVTEDLDIVSRGRTFPHGISKETAYAESVKLLQKILEEDERKIRRIGVRFSKFIEAIGLDKFFDT

SEQ ID NO: 127 - protein, 14 aa, Artificial sequence
  short peptide (translation of mRNA #7)
  MISC_FEATURE (1)..(1): fluorescein-labeled L-phenylalanine
  MISC_FEATURE (5)..(6): D-phenylalanine
FKKKFFDYKDDDDK

SEQ ID NO: 128 - protein, 15 aa, Artificial sequence
  short peptide (translation of mRNA #7)
FKKKFFFDYKDDDDK

SEQ ID NO: 129 - protein, 15 aa, Artificial sequence
  short peptide (translation of mRNA #7)
  MISC_FEATURE (1)..(1): fluorescein-labeled L-phenylalanine
  MISC_FEATURE (5)..(7): D-phenylalanine
FKKKFFFDYKDDDDK

SEQ ID NO: 130 - protein, 15 aa, Artificial sequence
  short peptide (translation of mRNA #10)
  MISC_FEATURE (1)..(1): fluorescein-labeled L-phenylalanine
  MISC_FEATURE (5)..(7): betta-amino acid
FKKKQQQDYKDDDDK

(19) United States
(12) Patent Application Publication
(10) Pub. No.: US 2025/0263696 A1
(43) Pub. Date: Aug. 21, 2025

(54) PROTEIN TRANSLATION SYSTEM

(71) Applicant: Tsinghua University, Beijing (CN)

(72) Inventors: Ting ZHU, Beijing (CN); Ji CHEN, Beijing (CN); Mengyin CHEN, Beijing (CN)

(73) Assignee: Tsinghua University, Beijing (CN)

(21) Appl. No.: 18/277,438

(22) PCT Filed: Feb. 17, 2022
(86) PCT No.: PCT/IB2022/051419

§ 371 Date: Aug. 16, 2023

Related U.S. Application Data
(60) Provisional application No. 63/150,641, filed on Feb. 18, 2021.

(51) Int. Cl. C12N 15/113; C12N 9/00; C12N 9/02; C12N 9/36

(52) U.S. Cl. CPC C12N 15/113; C12N 9/0069; C12N 9/2462; C12N 9/93; C12Y 113/12005; C12Y 302/01017; C12Y 601/01002; C12N 2310/12

(57) ABSTRACT

Provided herein is a cell-free and aaRS-free protein translation systems, and uses thereof in the production of proteins and active enzymes.

### RELATED APPLICATIONS

This application claims the benefit of priority of U.S. Provisional Patent Application No. 63/150,641 filed on 18 Feb. 2021, the contents of which are incorporated herein by reference in their entirety.

### SEQUENCE LISTING STATEMENT

The ASCII file, entitled 96672ReplacementSequenceListing.txt, created on Jan. 26, 2024, comprising 36,517 bytes, is incorporated herein by reference.

### FIELD AND BACKGROUND OF THE INVENTION

The present invention, in some embodiments thereof, relates to a cell-free protein translation system and more particularly, but not exclusively, to aminoacyl-tRNA synthetase-free methods of synthesizing proteins and their mirror-image counterparts, and uses thereof.

Cell-free protein synthesis is an important tool for molecular biologists in basic and applied sciences. It is increasingly being used in high-throughput functional genomics and proteomics, with significant advantages compared to protein expression in live cells.Cell-free protein synthesis is essential for the generation of protein arrays, such as nucleic acid programmable protein array (NAPPA) and enzyme engineering using display technologies. The cell-free approach provides the fastest way to correlate phenotype (function of expressed protein) to genotype. Protein synthesis can be performed in a few hours using either mRNA template in translational systems or DNA template (plasmid DNA or PCR fragments) in coupled transcription and translation systems. Furthermore, cell-free protein expression systems are indispensable for the expression of toxic proteins, membrane proteins, viral proteins and for proteins that undergo rapid proteolytic degradation by intracellular proteases.

Most cell-free protein expression are based on lysates, which are generated from cells engaged in a high rate of protein synthesis. The most frequently used cell-free expression systems require the macromolecular components for translation, such as ribosomes, tRNAs, aminoacyl-tRNA synthetases, initiation, elongation and termination factors. To ensure efficient translation, commercial extracts have to be supplemented with amino acids, energy sources (ATP, GTP), energy regenerating systems and salts (Mg2+, K+, etc.). For eukaryotic systems creatine phosphate and creatine phosphokinase serve as energy regenerating system, whereas prokaryotic systems are supplemented with phosphoenol pyruvate and pyruvate kinase. Coupled transcription and translation systems are supplemented with phage-derived RNA polymerase allowing the expression of genes cloned downstream of the polymerase promoter.

The emergence of protein enzymes is key to the transition from RNA-based life to contemporary biology. The discovery of tRNA-aminoacylation ribozymes suggested the possibility of synthesizing protein enzymes from highly simplified translation systems with tRNAs charged by ribozymes. Meanwhile, other systems using pre-charged tRNAs prepared by aaRS, urzymes, and chemical acylation have also been reported. Among them, a highly robust and versatile tRNA-aminoacylating ribozyme system, named the flexizyme, discovered through in vitro selection has been shown capable of charging a wide variety of amino acids to tRNAs. With tRNAs charged by flexizyme and aaRS, incorporation of multiple unnatural amino acids into translated peptides was achieved, enabling the practical selection of peptide drugs. However, in part due to the low translation yield, when using exclusively flexizyme-charged tRNAs in the absence of aaRS (hereinafter referred to as “aaRS-free”), only short peptides were translated (less than 7 amino-acid residues long), whereas the ribosomal production of full-length, functional protein enzymes with all 20 proteinogenic amino acids under aaRS-free conditions has remained undemonstrated thus far.

Terasaka, N. et al. [Terasaka, N., Hayashi, G., Katoh, T., and Suga, H. (2014). An orthogonal ribosome-tRNA pair via engineering of the peptidyl transferase center. Nat. Chem. Biol. 10, 555-557]report an engineered system using pairs of rRNAs and tRNAs with the compensatory mutations, which specifically uses a genetic code that is programmed distinctly from the naturally occurring genetic code and so is able to synthesize peptides orthogonally to the wild-type counterpart. By means of these translation machineries, a single mRNA produces two different peptides according to the artificially programmed genetic codes.

### SUMMARY OF THE INVENTION

Aspects of the present invention are drawn to cell-free and aaRS-free protein translation/expression/synthesis systems and methods, and uses thereof. The present disclosure provides a successful translation of multiple proteins, including active enzymes with distinct functions, using exclusively flexizyme-charged tRNAs, through improving the translation yield by reducing Mg²⁺concentration and increacing tRNAs concentration. Demonstrated is an aaRS-free translation system that produces an active aaRS (TrpRS), which in turn catalyzed the charging of more tRNAs. Also demonstrated in a mirror-image tRNA charged with D-amino acids by a synthetic L-flexizyme. The present disclosure demonstrates the feasibility of translating protein enzymes from a highly simplified translation apparatus without aaRS, and relaxes the requirement to chemically synthesize dozens of large aaRS proteins for realizing mirror-image translation. The cation-depleted flexizyme-charged tRNAs is useful in the translation of complete or partial unnatural peptides when used in conjunction with or without other aaRS proteins.

Thus, according to an aspect of some embodiments of the present invention there is provided a system for producing a protein, which includes:
an mRNA molecule encoding the protein;
a plurality of charged tRNA molecules; and
a cell-free translation mix,
wherein a concentration of Mg⁺² in the system is less than 100 mM.

According to some embodiments, the system is essentially devoid of an aminoacyl tRNA synthetase.

According to some embodiments, the concentration of the charged tRNA molecules is greater than 60 μM.

According to some embodiments, the concentration of the charged tRNA molecules is more than 160 μM and the concentration of Mg⁺² is more less than 100 mM.

According to some embodiments, the at least one tRNA molecule of the plurality of charged tRNA molecules is charged by a flexizyme.

According to some embodiments, the tRNA molecule is charged with an unnatural amino acid residue.

According to some embodiments, the unnatural amino acid residue is a D-amino acid residue.

According to some embodiments, the tRNA molecule comprises L-ribonucleic acid residues (L-tRNA).

According to some embodiments, the L-tRNA is prepared using a D-polymerase.

According to some embodiments, the D-polymerase is a mirror-image protein of Dpo4 (D-Dpo4).

According to some embodiments, the D-Dpo4 is D-Dpo4-5m-Y12S (SEQ ID No. 126).

According to some embodiments, the flexizyme comprises L-ribonucleic acid residues (L-flexizyme).

According to some embodiments, the protein is selected from the group consisting of an active L-protein enzyme, and an active D-protein enzyme.

According to another aspect of some embodiments of the present invention, there is provided a method of producing a protein using the system provided herein, the method includes: providing a plurality of charged tRNA molecules having no more than the concentration of Mg⁺²; and contacting the charged tRNA molecules with an mRNA molecule encoding a protein in a cell-free translation mix, to thereby obtain the protein.

According to some embodiments, the system used in the method is essentially devoid of an aminoacyl tRNA synthetase.

According to some embodiments, providing a plurality of charged tRNA molecules includes, prior to the contacting step, adjusting (lowering or depleting) the concentration of Mg⁺².

According to some embodiments, adjusting the concentration of Mg⁺² includes using a technique such as, for example, chromatography, alcohol precipitation and pellet washing, ultrafiltration and dialysis.

According to some embodiments, providing a plurality of charged tRNA molecules includes further includes adjusting the concentration of the charged tRNA molecules to a concentration greater than 2-fold of a charged tRNA concentration in other protein translation systems that include aaRS enzyme(s).

According to some embodiments, the concentration of the charged tRNA molecules is more than 160 μM.

According to another aspect of some embodiments of the present invention, there is provided a method of charging an L-tRNA with a D-amino acid, the method is effected by:
preparing the L-tRNA molecule using a D-polymerase;
providing an activated D-amino acid;
providing an L-aminoacylation ribozyme; and
contacting the L-tRNA, the L-aminoacylation ribozyme and the activated D-amino acid to thereby obtain a D-amino acid-charged L-tRNA molecule.

According to some embodiments, the L-aminoacylation ribozyme is an L-flexizyme.

According to some embodiments, the method can be analyzed by a PAGE analysis of the reaction mixture of the D-amino acid-charged L-tRNA molecule, wherein the PAGE gel is characterized by a distinct peak for a charged tRNA species and a distinct peak for an uncharged tRNA species.

According to another aspect of some embodiments of the present invention, there is provided an L-flexizyme that includes L-ribonucleotide residues.

In some embodiments, the L-flexizyme includes at least 40%, 50%, 60%, 70%, 80%, or 90% L-ribonucleotide residues.

In some embodiments, the L-flexizyme consists of L-ribonucleotide residues.

In some embodiments, the L-flexizyme is having a sequence that exhibits at least 80% identity to 5′-ggaucgaaagauuuccgcauccccgaaaggguacauggcguuaggu-3′(SEQ ID No. 82).

According to another aspect of some embodiments of the present invention, there is provided a protein prepared by the method provided herein.

In some embodiments, the protein is selected from the group consisting of a protein that comprises at least one non-canonical amino acid residue, a protein that comprises at least one D-amino acid residue, an L-protein and a D-protein.

In some embodiments, the protein is selected from the group consisting of chicken lysozyme, Gaussia luciferase, and E. coli TrpRS.

In some embodiments, the protein is having a sequence that can be decoded into textual and/or numerical information, and comprising natural amino acids and/or unnatural amino acids.

In some embodiments, the protein is encoded by mRNA #6.

According to another aspect of some embodiments of the present invention, there is provided a library of randomized or partially randomized peptides, obtained by the method provided, wherein at least of the peptides comprise at least one unnatural amino acid.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying figures. With specific reference now to the figures in detail, it is stressed that the particulars shown are by way of example and for purposes of illustrative discussion of embodiments of the invention. In this regard, the description taken with the figures makes apparent to those skilled in the art how embodiments of the invention may be practiced.

In the figures:

FIG. 1 presents a schematic overview illustration of some aspects of the present invention, and in particular an aaRS-free translation of proteins using flexizyme-charged tRNAs (10), wherein tRNAs 11 are charged by flexizyme system 12, generating a population of charged tRNAs 13 representing proteinogenic amino acids for the translation of protein enzymes, and including step 14a wherein charged tRNAs are purified by HPLC to reduce Mg²⁺, 14b contamination, and including step 15 wherein charged tRNAs 13 are concentrated for aaRS-free translation of mRNA 16 in ribosome 17 a translated polypeptide 18 that can fold into active protein enzymes 19a including aaRS 19b, which can be used to charge tRNAs to complete the cycle;

FIG. 2. Presents an acid PAGE analysis of tRNA charging yields before and after HPLC purification, wherein “U” represents uncharged tRNA, “C” represents crude charged tRNA, “P” represents purified charged tRNA, whereas the tRNA charging yields were determined by software package IMAGEJ using the integrated peak area of charged tRNAs relative to the total tRNAs;

FIGS. 3A-E present concept and results of flexizyme charging of tRNAs en route to an aaRS-free charging of mirror-image tRNAs, according to some embodiments of the present invention, showing D-tRNA charging catalyzed by D-flexizyme, and its mirror-image version, mirror-image tRNA charging catalyzed by L-flexizyme (PDB sources: 1EHZ (tRNA), 3CUL (flexizyme)(FIG. 3A), L-flexizyme charging of D-alanine onto enzymatically transcribed mirror-image tRNA^{ALa}, with the natural-chirality counterparts shown for comparison (FIG. 3B), L-flexizyme charging of glycine onto enzymatically transcribed mirror-image tRNAGly, with the natural-chirality counterparts shown for comparison (FIG. 3C) L-flexizyme charging of D-lysine onto enzymatically transcribed mirror-image tRNA^{LYs}, with the natural-chirality counterparts shown for comparison (FIG. 3D), L-flexizyme charging of D-phenylalanine onto enzymatically transcribed mirror-image tRNA^{Phe}, with the natural-chirality counterparts shown for comparison (FIG. 3E), whereas the tRNA charging yields were determined using software package IMAGEJ using the integrated peak area of charged tRNAs relative to the total tRNAs;

FIGS. 4A-G present the results of an aaRS-free translation of multiple short peptides, according to some embodiments of the present invention, showing MALDI-TOF-MS analysis of translated short peptides from mRNA #1 (FIG. 4A), aaRS-free translation yield of short peptides, analyzed by Tricine-SDS-PAGE, showing uncharged tRNA concentrations ranged from 160-540 M while the flexizyme-charged tRNA concentration remained at 70 M, resulting in charging yields ranging from 44-13% (upper part of FIG. 4B), and total tRNA concentrations ranged from 16-1003 μM while the charging yield remained at 56% (lower part of FIG. 4B)(error bar represents standard deviations from three independent experiments), MALDI-TOF-MS analysis of translated short peptides from mRNA #2 (FIG. 4C), mRNA #3 (FIG. 4D), mRNA #4 (FIG. 4E), mRNA #5 (FIG. 4F), and mRNA #6 (FIG. 4G);

FIGS. 5A-D present results of aaRS-free translation of mRNA #1 under various conditions, showing total tRNA concentrations ranged from 20-644 μM, with charging yield remained at 44% (FIG. 5A), total flexizyme concentrations ranged from 240-525 μM, with total tRNA concentration remained at 160 M (FIG. 5B), flexizyme and uncharged tRNAs from 0-380 μM were mixed in (FIG. 5C) 10 mM MgCl2 and (FIG. 5D) 100 mM MgCl2, desalted by ethanol precipitation, and added to the aaRS-free translation mix, wherein the concentration of flexizyme-charged tRNA was remained at 70 M (error bar, standard deviations from three independent experiments);

FIGS. 6A-E present tricine-SDS-PAGE gel analysis for calculating the aaRS-free translation yields, showing gel images corresponding to FIG. 4B, FIG. 5A, FIG. 5B, FIG. 5C, and FIG. 5D (FIGS. 6A-E respectively), for calculating the aaRS-free translation yields, wherein “M” is a synthetic peptide standard (Fph-K-Y-D-K-Y-D (SEQ ID No. 125));

FIGS. 7A-B presents the results of an in vitro translation experiment in the presence of LysRS, TyrRS and AspRS, showing tricine-SDS-PAGE analysis of translation products with uncharged, unmodified total tRNA concentrations ranging from 22-680 μM in the presence of LysRS, TyrRS and AspRS, and Fph-tRNAfMet pre-charged by enhanced flexizyme (FIG. 7A, and the calculated translation yield (FIG. 7B)(error bar, standard deviations from three independent experiments);

FIGS. 8A-B present flexizyme-charging yields of 21 tRNAs with their cognate proteinogenic amino acids, showing the charging yield determined after ethanol precipitation (FIG. 8A), and the charging yield determined after HPLC purification of 14 flexizyme-charged tRNAs. N/A, purification of flexizyme-charged tRNAs not performed (FIG. 8B);

FIG. 9 presents MALDI-TOF MS analysis of aaRS-free translated mRNA #6, showing that with a higher total tRNA concentration (520 μM) in the aaRS-free translation system, a mistranslated product was observed with a M.W. of 2,252.7 Da, whereas the correctly translated product had a M.W. of 2,240.7 Da. a.u., arbitrary units; C, O: calculated and observed m/z values, respectively;

FIGS. 10A-C present the amino acid sequences of aaRS-free translated protein enzymes: chicken lysozyme (FIG. 10A), Gaussia luciferase (FIG. 10B), and E. coli TrpRS (FIG. 10C), whereas positions translated by the flexizyme-charged tRNAs were purified either by ethanol precipitation or by HPLC (underlined);

FIGS. 11A-G present SDS-PAGE analysis of aaRS-free translated protein enzymes, showing the entire gel image shown in FIG. 12A (FIG. 11A), a samples of 400 ng commercial chicken lysozyme purified from chicken egg white that were analyzed in 15% SDS-PAGE, and stained by Coomassie Brilliant Blue (FIG. bIB), the entire gel image shown in FIG. 12C (FIG. 11C), samples of 400 ng recombinant Gaussia luciferase, expressed and purified from E. coli strain BL21 that were analyzed 15% SDS-PAGE, and stained by Coomassie Brilliant Blue (FIG. bID), the entire gel image shown in FIG. 14A (FIG. 11E), samples of 300 ng recombinant E. coli TrpRS, expressed and purified from E. coli strain BL21 that were analyzed 15% SDS-PAGE, and stained by Coomassie Brilliant Blue (FIG. 11F), and samples of 5 M Fph-CME, 1 M Fph-tRNA^{fMet}, and 5 μM of Fph-tRNA^{fMet} that were analyzed by 15% SDS-PAGE with or without being heated to 98° C. for 3 min, and scanned by Typhoon FLA 9500 under Cy2 mode (FIG. 11G), wherein M is a benchmark fluorescent protein standard;

FIGS. 12A-D present results of experimental proof of concept of aaRS-free translation of protein enzymes, according to some embodiments of the present invention, showing aaRS-free translation of N-terminal FAM-labeled chicken lysozyme, analyzed by 15% SDS-PAGE, and scanned by Typhoon FLA 9500 under Cy2 mode (M represents a benchmark fluorescent protein standard) (FIG. 12A), enzymatic assay of crude aaRS-free translated chicken lysozyme, with fluorescently labeled bacterial (Micrococcus lysodeikticus) cell wall materials as substrates (FIG. 12B), aaRS-free translation of N-terminal FAM-labeled Gaussia luciferase, analyzed by 15% SDS-PAGE, and scanned by Typhoon FLA 9500 under Cy2 mode (FIG. 12C), and enzymatic assay of crude aaRS-free translated Gaussia luciferase, with coelenterazine as substrate (FIG. 12D)(RFU, relative fluorescence unit. RLU, relative luminescence unit);

FIG. 13 presents yield estimate values of aaRS-free translated Gaussia luciferase, wherein the standard curve plotted using 0, 25 nM, 50 nM, 100 nM, and 250 nM recombinant Gaussia luciferase (denoted by squares), and the yield of the translated Gaussia luciferase was estimated to be −25 nM (denoted by a triangle);

FIGS. 14A-C presents aaRS-free translation of TrpRS, showing aaRS-free translation of N-terminal FAM-labeled E. coli TrpRS, analyzed by 15% SDS-PAGE, and scanned by Typhoon FLA 9500 under Cy2 mode (M represents a benchmark fluorescent protein standard (FIG. 14A), sequence and secondary structure of internally Cy5-labeled tRNA^{Trp} (FIG. 14B), and enzymatic assay of crude aaRS-free translated TrpRS, with Cy5-tRNA^{Trp} as substrate, analyzed by 8% acid PAGE, and scanned by Typhoon FLA 9500 under Cy5 mode (FIG. 14C);

FIGS. 15A-B present results of the transcription of mirror-image tRNA^{Lys} by D-Dpo4-5m-Y12S, showing the extension of a 5′-FAM labeled L-universal primer on an L-ssDNA template, polymerized by the synthetic D-Dpo4-5m-Y12S polymerase, and the reaction aliquots that were terminated at different time points and analyzed by 12% denaturing PAGE gel in 7 μM urea (FIG. 15A), and showing mirror-image transcription and I2-mediated cleavage of the tRNA^{LYs} transcript, analyzed by 10% denaturing PAGE gel in 7 μM urea, and stained by SYBR-Green II by Thermo Fisher Scientific, MA, U.S. (FIG. 15B);

FIGS. 16A-B present results of the biochemical characterization of enzymatically transcribed natural and mirror-image tRNAs, showing RNase A digestion of enzymatically transcribed D- and L-tRNA^{Ala} (FIG. 16A), and AaRS-catalyzed aminoacylation of enzymatically transcribed D- and L-tRNA^{Ala} (FIG. 16B);

FIGS. 17A-C present MALDI-TOF MS analysis of I2-mediate cleavage, showing synthetic DNA-RNA chimeric oligo cleaved at the phosphorothioate modification site by 12 (FIG. 17A), MALDI-TOF MS spectrum of the uncleaved oligo under negative linear mode (FIG. 17B), MALDI-TOF MS spectrum of I2-cleaved oligo under negative linear mode (m/z >4000) and negative reflectron mode (m/z<4000) (FIG. 17C), wherein the upper-case letters denote DNA nucleotides, lower-case letters denote RNA nucleotides, “*” denotes phosphorothioate modification. a.u., arbitrary units; C, O, calculated and observed m/z values, respectively;

FIGS. 18A-B present translation of complete or partial unnatural peptides using cation-depleted flexizyme-charged tRNAs, showing translation of peptide drugs and unnatural proteins using the cation-depleted flexizyme-charged tRNAs in in vitro translation systems (FIG. 18A), and translation of complete or partial unnatural proteins, data storage, and ribosome/mRNA display using the cation-depleted flexizyme-charged tRNAs in in vitro translation systems (FIG. 18B);

FIGS. 19A-B present 8% acid PAGE photographs and analysis of the experimental proof-of-concept of charging fully functional L-tRNA molecules, which was enzymatically transcribed by a mirror-image enzyme (D-Dpo4-5m-Y12S), with pre-activated amino-acids, wherein FIG. 19A shows the results charging enzymatically transcribed L-tRNA and FIG. 19B shows the results charging synthetically generated L-tRNA;

FIGS. 20A-C present the result of the in vitro translation of a short peptide containing two consecutive D-phenylalanine, wherein FIG. 20A shows MALDI-TOF-MS analysis of translated short peptides from mRNA #7, FIG. 20B shows MALDI-TOF-MS analysis of translated short peptides from mRNA #8, and FIG. 20C shows Tricine-SDS-PAGE analysis of translation products of mRNA #7 or mRNA #8 with uncharged tRNAPhe only (mRNA #7), 20 μM LPhe-tRNAPhe (mRNA #7), 20 μM DPhe-tRNAGluE2CUA (mRNA #8), or 200 μM DPhe-tRNAGluE2CUA (mRNA #8), scanned by Typhoon FLA 9500 under Cy2 mode;

FIGS. 21A-B present the result of the in vitro translation of a short peptide containing three consecutive D-phenylalanine, wherein FIG. 21A shows MALDI-TOF-MS analysis of translated short peptides from mRNA #9, and FIG. 21B shows Tricine-SDS-PAGE analysis of translation products of mRNA #9 with uncharged tRNAPhe only, 30 μM LPhe-tRNAPhe, 30 μM DPhe-tRNAGluE2CUA, or 300 μM DPhe-tRNAGluE2CUA, scanned by Typhoon FLA 9500 under Cy2 mode; and FIG. 22 presents the results of the in vitro translation of a short peptide containing three consecutive 3-Gln, showing the Tricine-SDS-PAGE analysis of translation products of mRNA #10 with uncharged tRNA only, 30 μM PGln-tRNAGluE2CUA, or 300 μM PGln-tRNAGluE2_{CUA}, scanned by Typhoon FLA 9500 under Cy2 mode.

### DESCRIPTION OF SOME SPECIFIC EMBODIMENTS OF THE INVENTION

The present invention, in some embodiments thereof, relates to a cell-free protein translation system and more particularly, but not exclusively, to aminoacyl-tRNA synthetase-free methods of synthesizing proteins and their mirror-image counterparts, and uses thereof.

The principles and operation of the present invention may be better understood with reference to the figures and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As discussed hereinabove, despite the discovery of tRNA-aminoacylating ribozymes such as the flexizyme, synthesizing protein enzymes from highly simplified translation systems in the absence of aaRS remains undemonstrated. One of the main reasons for the low yield of aaRS-free translation is that, compared with tRNA aminoacylation by aaRS, the flexizyme-charging of tRNAs lacks recycling. In addition, the use of in vitro transcribed, unmodified tRNAs for aaRS-free charging may also contribute to the low translation yield.

While conceiving the present invention, the inventors set out to test the ability of aaRS-free systems to translate protein enzymes with all 20 proteinogenic amino acids using tRNAs charged exclusively by the flexizyme. The preliminary results showed that, with increasing the concentration of flexizyme-charged tRNAs and reducing the concentration of the cation Mg2+by purification, multiple protein enzymes of distinct functions such as the lysozyme, luciferase, and even aaRS itself can be synthesized. Charging of mirror-image L-tRNAs with mirror-image D-amino acids by a synthetic mirror-image L-flexizyme, has been demonstrated as well, which eventually enables the realization of a mirror-image translation apparatus.

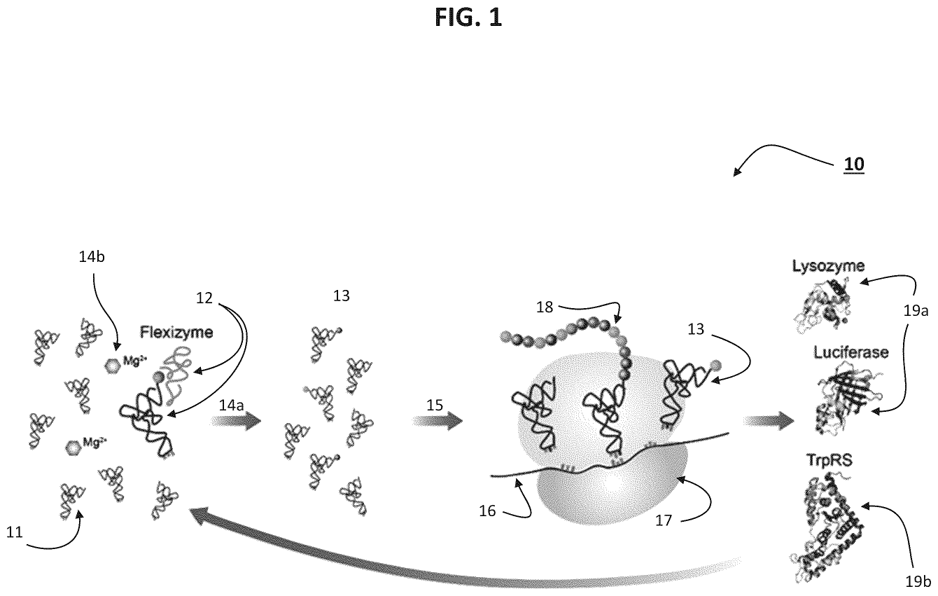

FIG. 1 presents a schematic overview illustration of some aspects of the present invention, and in particular an aaRS-free translation of proteins using flexizyme-charged tRNAs (10), wherein tRNAs 11 are charged by flexizyme system 12, generating a population of charged tRNAs 13 representing proteinogenic amino acids for the translation of protein enzymes, and including step 14 wherein charged tRNAs are purified by HPLC to reduce Mg²⁺contamination, and including step 15 wherein charged tRNAs 13 are concentrated for aaRS-free translation of mRNA 16 in ribosome 17 a translated polypeptide 18 that can fold into active protein enzymes 19a including aaRS 19b, which can be used to charge tRNAs to complete the cycle.

Demonstrated herein is the aaRS-free translation of protein enzymes with an exclusive set of ribozyme-charged tRNAs. Shown is a finding that neither aaRS-catalyzed tRNA charging nor tRNA recycling are required for processive and faithful ribosome translation which led to the revelation that protein enzymes, possessing more structural motifs and hence more catalytic functions than short peptides, could be translated from the highly simplified aaRS-free translation system. Notably, the average size of modem natural proteins is about 270-470 aa. The aaRS-free translation of proteins as large as TrpRS suggests the possibility of producing other important protein enzymes, such as the tRNA modifying enzymes, to further improve the translation efficiency and fidelity. Also shown herein is the discovery that high concentrations of ribozyme-charged tRNAs greatly improves the yield of aaRS-free translation may shed light on the possible conditions for the emergence of protein enzymes on prebiotic earth, where abundant feedstocks of ribozyme-charged tRNAs might be important for primitive translation systems to operate efficiently.

One of the limitations of the current aaRS-free translation system is that the charging of tRNAs must be decoupled from translation in that they were pre-charged before being added to the translation system, since the flexizyme is a non-specific catalyst that charges various amino acids to tRNAs. The methodology of using high concentrations of flexizyme-charged tRNAs and removal of Mg2+contamination by purification, which is shown to have greatly improved the yield of aaRS-free translation, can be applied to other in vitro translation systems using pre-charged tRNAs (with or without aaRS) for producing peptides or proteins from all or partial unnatural amino acids, enabling immediate applications in many fields of synthetic biology and drug discovery.

The realization of aaRS-free translation of protein enzymes establishes a path to a translation apparatus without any aaRS, as a more feasible model for realizing mirror-image translation, since all the aaRS proteins combined represent 29% (about 1.4 μMDa) in molecular weight of the E. coli translation apparatus including the ribosome, translation factors, aaRSs, and tRNAs (with a total molecular weight of about 4.9 μMDa). Moreover, the translation of the small 169-aa Gaussia lucifierase, demonstrated herein, provides a sensitive and chiral-specific assay for testing mirror-image translation.

AaRS-free cell-free translation system:

As discussed hereinabove, cell-free protein synthesis offers a facile and rapid method for synthesizing, monitoring, analyzing, and purifying proteins from a DNA template, and at the same time open the path to genetic code expansion methods that inter-alia allow site-specifically incorporation of unnatural amino acids (UAAs; also known as noncanonical amino acids) into proteins via ribosomal translation. While known systems are based on the exogenous addition of an orthogonal translation system (OTS), comprising an orthogonal tRNA, and orthogonal aminoacyl tRNA synthetase (aaRS), to the cell-free reaction mixture, the herein-provided protein translation system expands this concept even further, by permitting the efficient production of proteins without the presence of any aminoacyl tRNA synthetase (aaRS), hence an aaRS-free translation system is provided herein.

In the context of the present disclosure, the term “aaRS-free”, as used herein, refers to a ribosomal translation system and/or method and/or platform for preparing proteins from a transcription template (e.g., ribonucleic acid molecule), that is essentially devoid of an aminoacyl tRNA synthetase (aaRS). By essentially devoid of an aminoacyl tRNA synthetase, it is meant that none of the steps of the protein production involves the use or the presence of an aaRS. The only exception to the definition of an aaRS-free translation system/method/platform, according to some embodiments of the present invention, is the embodiment wherein an aminoacyl tRNA synthetase is the protein product that is being produced thereby. By being essentially devoid of any tRNA synthetase enzyme, it is meant that the system does not include the means to charge amino acid residues to tRNA, and that aaRS enzymes are not introduced into the system at any stage of the translation, and that the entire supply of amino acid residues comes from pre-charged tRNA molecules.

Thus, according to an aspect of some embodiments of the present invention, there is provided a system for producing a protein, which includes:
an mRNA molecule encoding the protein;
a plurality of charged tRNA molecules; and
an cell-free translation mix,
   wherein the system is essentially devoid of an aminoacyl tRNA synthetase, a concentration of Mg⁺² in the system is lower than 100 mM.

The term “system”, as used herein, refers to a reaction mixture (i.e., solvent, solutes, reactants, and optional detection markers) and reaction conditions (concentrations, temperature, and mixing) which are conducive and essential for effecting a complex chemical reaction such as protein synthesis.

In the context of some embodiments of the present invention, the phrase “a cell-free translation mix” refers to an in vitro protein translation mixture that does not involve the use of intact/viable cells, and includes ribosomes and ribosomal translation factors that are essential for cell-free in vitro protein translation reaction, as these terms are known in the art. In the context of some embodiments of the present invention, the phrase “aaRS-free translation mix”, refers to a cell-free translation mix, as known in the art, with the exception that the cell-free (in vitro) translation mix is essentially devoid of aaRS proteins, unless stated otherwise.

The protein translation system includes a messenger RNA molecule that encodes the amino-acid sequence of the desired protein to be produced by the system. Alternatively, the system may include the means to transcribe a DNA template into the mRNA molecule, namely a DNA template and the transcription factors to effect DNA-to-RNA transcription (e.g., RNA nucleotides, RNA polymerase and general transcription factors).

The protein translation system includes a plurality of charged tRNA molecules, which are also referred to herein in the context of some embodiments of the invention, as pre-charged tRNA transcripts. In some embodiments the tRNA molecules are synthetically prepared polynucleotides, and in other embodiments the tRNA molecules are enzymatically prepared transcripts, and the relevant differences between the two categories are discussed hereinbelow.

According to some embodiments of the present invention, this plurality of charged tRNA molecules includes at least tRNA molecules that are charged with amino acid residues that are encoded for by the mRNA, and are going to be present in the protein sequence, as encoded by the mRNA molecule. The plurality of charged tRNA molecules also includes tRNA molecules charged with unnatural amino acid residues, including residues of D-amino acids and other non-canonical amino acid residues, as presented in Tables A and B below. Preferably, the frequency and amount of each of the individual charged tRNA molecules matches the frequency of each amino acid in the sequence of the protein. For example, if the frequency of serine residues in the protein sequence is 8%, and the frequency of methionine is 1%, the plurality of charged tRNA molecules in the system will reflect that frequency, and include about eight-times more tRNAs^{er}than tRNA^{Met}. In some embodiments, the tRNA molecules are made of L-nucleotides, rendering the tRNA molecules mirror-images of naturally occurring tRNA molecules. In some embodiments, the tRNA molecules are made of L-nucleotides and further charged with residues of D-amino acids.

As used herein, the terms “residue” and/or “moiety” describe a portion of a molecule, and typically a major portion thereof, or a group of atoms pertaining to a specific function. For example, the term “amino-acid residue” refers to an amino-acid in the context of a compound having an amino-acid attached thereto; a peptide is a chain of amino-acid residues linked to one-another; a tRNA molecule charged with a ribonucleic acid residue is a ribonucleic acid attached to a tRNA molecule.

Tables A-B present some of the optional amino acid residues that are relevant in the context of some embodiments of the present invention; noted, these are examples, and should not be seen as limiting.

TABLE A

| | Three-Letter | One-letter |
|---|---|---|
| Amino acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Iie | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

TABLE B

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane-carboxylate | Cpro | L-N-methylasparagine | Nmasn |
| aminoisobutyric acid | Aib | L-N-methylaspartic acid | Nmasp |
| aminonorbornyl-carboxylate | Norb | L-N-methylcysteine | Nmcys |
| Cyclohexylalanine | Chexa | L-N-methylglutamine | Nmgin |
| Cyclopentylalanine | Cpen | L-N-methylglutamic acid | Nmglu |
| D-alanine | Dal | L-N-methylhistidine | Nmhis |
| D-arginine | Darg | L-N-methylisolleucine | Nmile |
| D-aspartic acid | Dasp | L-N-methylleucine | Nmleu |
| D-cysteine | Dcys | L-N-methyllysine | Nmlys |
| D-glutamine | Dgln | L-N-methylmethionine | Nmmet |
| D-glutamic acid | Dglu | L-N-methylnorleucine | Nmnle |
| D-histidine | Dhis | L-N-methylnorvaline | Nmnva |
| D-isoleucine | Dile | L-N-methylornithine | Nmorn |
| D-leucine | Dleu | L-N-methylphenylalanine | Nmphe |
| D-lysine | Dlys | L-N-methylproline | Nmpro |
| D-methionine | Dmet | L-N-methylserine | Nmser |
| D/L-ornithine | D/Lorn | L-N-methylthreonine | Nmthr |
| D-phenylalanine | Dphe | L-N-methyltryptophan | Nmtrp |
| D-proline | Dpro | L-N-methyltyrosine | Nmtyr |
| D-serine | Dser | L-N-methylvaline | Nmval |
| D-threonine | Dthr | L-N-methylethylglycine | Nmetg |
| D-tryptophan | Dtrp | L-N-methyl-t-butylglycine | Nmtbug |
| D-tyrosine | Dtyr | L-norleucine | Nle |
| D-valine | Dval | L-norvaline | Nva |
| D-α-methylalanine | Dmala | α-methyl-aminoisobutyrate | Maib |
| D-α-methylarginine | Dmarg | α-methyl-□-aminobutyrate | Mgabu |
| D-α-methylasparagine | Dmasn | α-methylcyclohexylalanine | Mchexa |
| D-α-methylaspartate | Dmasp | α-methylcyclopentylalanine | Mcpen |
| D-α-methylcysteine | Dmcys | α- methylα-naphthylalanine | Manap |
| D-α-methylglutamine | Dmgln | α-methylpenicillamine | Mpen |
| D-α-methylhistidine | Dmhis | N-(4-aminobutyl)glycine | Nglu |
| D-α-methylisoleucine | Dmile | N-(2-aminoethyl)glycine | Naeg |
| D-α-methylleucine | Dmleu | N-(3-aminopropyl)glycine | Norn |
| D-α-methyllysine | Dmlys | N-amino-a-methylbutyrate | Nmaabu |
| D-α-methylmethionine | Dmmet | □-napthylalanine | Anap |
| D-α-methylornithine | Dmorn | N-benzylglycine | Nphe |
| D-α-methylphenylalanine | Dmphe | N-(2-carbamylethyl)glycine | Ngln |
| D-α-methylproline | Dmpro | N-(carbamylmethyl)glycine | Nasn |
| D-α-methylserine | Dmser | N-(2-carboxyethyl)glycine | Nglu |
| D-α-methylthreonine | Dmthr | N-(carboxymethyl)glycine | Nasp |
| D-α-methyltryptophan | Dmtrp | N-cyclobutylglycine | Ncbut |
| D-α-methyltyrosine | Dmty | N-cycloheptylglycine | Nchep |
| D-α-methylvaline | Dmval | N-cyclohexylglycine | Nchex |
| D-α-methylalnine | Dnmala | N-cyclodecylglycine | Ncdec |
| D-α-methylarginine | Dnmarg | N-cyclododeclglycine | Ncdod |
| D-α-methylasparagine | Dnmasn | N-cyclooctylglycine | Ncoct |
| D-α-methylasparatate | Dnmasp | N-cyclopropylglycine | Ncpro |
| D-α-methylcysteine | Dnmcys | N-cycloundecylglycine | Ncund |
| D-N-methylleucine | Dnmleu | N-(2,2-diphenylethyl)glycine | Nbhm |
| D-N-methyllysine | Dnmlys | N-(3,3-diphenylpropyl)glycine | Nbhe |
| N-methylcyclohexylalanine | Nmchexa | N-(3-indolylyethyl) glycine | Nhtrp |
| D-N-methylornithine | Dnmorn | N-methyl-□-aminobutyrate | Nmgabu |
| N-methylglycine | Nala | D-N-methylmethionine | Dnmmet |
| N-methylaminoisobutyrate | Nmaib | N-methylcyclopentylalanine | Nmcpen |
| N-(1-methylpropyl)glycine | Nile | D-N-methylphenylalanine | Dnmphe |
| N-(2-methylpropyl)glycine | Nile | D-N-methylproline | Dnmpro |
| N-(2-methylpropyl)glycine | Nleu | D-N-methylserine | Dnmser |
| D-N-methyltryptophan | Dnmtrp | D-N-methylserine | Dnmser |
| D-N-methyltyrosine | Dnmtyr | D-N-methylthreonine | Dnmthr |
| D-N-methylvaline | Dnmval | N-(1-methylethyl)glycine | Nva |
| □-aminobutyric acid | Gabu | N- methylα-naphthylalanine | Nmanap |
| L-t-butylglycine | Tbug | N-methylpenicillamine | Nmpen |
| L-ethylglycine | Etg | N-(p-hydroxyphenyl)glycine | Nhtyr |
| L-homophenylalanine | Hphe | N-(thiomethyl)glycine | Ncys |
| L-α-methylarginine | Marg | penicillamine | Pen |
| L-α-methylaspartate | Masp | L-α-methylalanine | Mala |
| L-α-methylcysteine | Mcys | L-α-methylasparagine | Masn |
| L-α-methylglutamine | Mgln | L-α-methyl-t-butylglycine | Mtbug |
| L-α-methylhistidine | Mhis | L-methylethylglycine | Metg |
| L-α-methylisoleucine | Mile | L-α-methylglutamate | Mglu |
| D-N-methylglutamine | Dnmgln | L-α-methylhomo phenylalanine | Mhphe |
| D-N-methylglutamate | Dnmglu | N-(2-methylthioethyl)glycine | Nmet |
| D-N-methylhistidine | Dnmhis | N-(3-guanidinopropyl)glycine | Narg |
| D-N-methylisoleucine | Dnmile | N-(1-hydroxyethyl)glycine | Nthr |
| D-N-methylleucine | Dnmleu | N-(hydroxyethyl)glycine | Nser |
| D-N-methyllysine | Dnmlys | N-(imidazolylethyl)glycine | Nhis |
| N-methylcyclohexylalanine | Nmchexa | N-(3-indolylyethyl)glycine | Nhtrp |
| D-N-methylornithine | Dnmorn | N-methyl-□-aminobutyrate | Nmgabu |
| N-methylglycine | Nala | D-N-methylmethionine | Dnmmet |
| N-methylaminoisobutyrate | Nmaib | N-methylcyclopentylalanine | Nmcpen |
| N-(1-methylpropyl)glycine | Nile | D-N-methylphenylalanine | Dnmphe |
| N-(2-methylpropyl)glycine | Nleu | D-N-methylproline | Dnmpro |
| D-N-methyltryptophan | Dnmtrp | D-N-methylserine | Dnmser |
| D-N-methyltyrosine | Dnmtyr | D-N-methylthreonine | Dnmthr |
| D-N-methylvaline | Dnmval | N-(1-methylethyl)glycine | Nval |
| □-aminobutyric acid | Gabu | N-methyla- naphthylalanine | Nmanap |
| L-t-butylglycine | Tbug | N-methylpenicillamine | Nmpen |
| L-ethylglycine | Etg | N-(p-hydroxyphenyl)glycine | Nhtyr |
| L-homophenylalanine | Hphe | N-(thiomethyl)glycine | Ncys |
| L-α-methylarginine | Marg | penicillamine | Pen |
| L-α-methylaspartate | Masp | L-α-methylalanine | Mala |
| L-α-methylcysteine | Mcys | L-α-methylasparagine | Masn |
| L-α-methylglutamine | Mgln | L-α-methyl-t-butylglycine | Mtbug |
| L-α-methylhistidine | Mhis | L-methylethylglycine | Metg |
| L-α-methylisoleucine | Mile | L-α-methylglutamate | Mglu |
| L-α-methylleucine | Mleu | L-α-methylhomophenylalanine | Mhphe |
| L-α-methylmethionine | Mmet | N-(2-methylthioethyl)glycine | Nmet |
| L-α-methylnorvaline | Mnva | L-α-methyllysine | Mlys |
| L-α-methylphenylalanine | Mphe | L-α-methylnorleucine | Mnle |
| L-α-methylserine | mser | L-α-methylornithine | Morn |
| L-α-methylvaline | Mtrp | L-α-methylproline | Mpro |
| L-α-methylleucine | Mval Nnbhm | L-α-methylthreonine | Mthr |
| N-(N-(2,2-diphenylethyl)carbamylmethyl-glycine | Nnbhm | L-α-methyltyrosine | Mtyr |
| 1-carboxy-1-(2,2-diphenyl ethylamino)cyclopropane | Nmbc | L-N-methylhomophenylalanine | Nmhphe |
| N-(N-(3,3-diphenylpropyl)carbamylmethyl(1)glycine | Nnbhe | D/L-citrulline | D/Lctr |

Cation-depleted system:

As disclosed hereinabove, the cell-free aaRS-free system for producing proteins, according to embodiments of the present invention, is effective in low cation concentration, and more specifically, low magnesium ion concentration. Magnesium is present in relatively high concentration in most cell-free protein translation mixtures, including commercial mixtures.

Magnesium is also present in most charged tRNA preparations, particularly flexizyme-charged tRNA preparations. As presented in the Examples section that follows below, the present inventors have surprisingly found that reducing the magnesium concentration to a practical minimum in the cell-free aaRS-free protein translation system greatly improved the efficiency and fidelity of protein production. Therefore, the inherent presence of magnesium ions that is carried over from the various components in known protein translation systems had to be reduced purposefully by the inventors in order to arrive at the improved performance of the herein-disclosed system.

Thus, according to some embodiments of the present invention, the system for producing a protein is characterized by a low Mg⁺² concentration compared to any known cell-free protein translation system hitherto. More specifically, the magnesium concentration in the system, according to the present invention, is lower than the Mg⁺² concentration in the charged-tRNA preparation. In absolute values, the concentration of Mg⁺² in the system is lower than 100 mM, 90 mM, 80 mM, 70 mM, 60 mM, 50 mM, 40 mM, 30 mM, 20 mM or lower than 10 mM.

In some embodiments, the concentration of magnesium ions in the system is the minimal concentration that is practically possible to obtain by ion-depletion methods, such as, without limitation, chromatography (HPLC), precipitation in alcohol and pellet wash, ultrafiltration and dialysis.

Charged-tRNA concentration:

The tRNA molecules of the presently disclosed system may be pre-charged by any method known in the art, and in some preferred embodiments, the tRNA is charged by a flexizyme. The concentration of the charged-tRNA molecules that are present in system is also subject to modification, compared to their concentration in known cell-free protein translation systems.

According to some embodiments, the concentration of the charged-tRNA is at least 2-times higher than in other known cell-free protein translation system. According to some embodiments, the concentration of the charged-tRNA is greater than 50 μM, 60 μM, 80 μM, 90 μM, 100 μM, 110 μM, 120 μM, 130 μM, 140 μM, 150 μM, 160 μM, 170 μM, 180 μM, 190 μM, or greater than 200 μM.

Inverse chirality elements:

Since there is no requirement for aaRS enzymes in the system provided herein, the system is particularly suitable for translating protein with unnatural/non-canonical amino acid residues, and among these, D-amino acid residues. The system can be used to insert D-amino acid residues into any polypeptide chain, including the translation of an mRNA into an all D-aa chain. As demonstrated hereinbelow, the system has been used to translate a complete mirror image protein.

As presented hereinbelow, the system was used successfully with tRNA molecule that include or consist of L-ribonucleic acid residues (L-tRNA). Hence, according to some embodiments of the present invention, the system includes L-tRNA molecules. Without limitation, the L-tRNA is prepared using a D-polymerase, such as D-Dpo4-5m-Y12S; however, other methods of producing L-tRNA molecules are also contemplated within the scope of the present invention.

In some embodiments of the present invention, the system comprises L-tRNA molecules, pre-charged by L-flexizyme with D-amino acid residues, to translate a D-protein (a mirror image protein).

L-aminoacylation ribozyme:

According to some embodiments of the present invention, the system includes L-tRNA that are pre-charged by a ribozyme having an aminoacyl-tRNA synthetase (aaRS) activity, namely an aminoacylation ribozyme. In some embodiments, the aminoacylation ribozyme is a flexizyme. In some preferred embodiments, the L-tRNA is charged with an L-flexizyme, which is a ribozyme made entirely or substantially from L-ribonucleotides.

Thus, according to an aspect of some embodiments of the present invention, there is provided a polyribonucleic acid molecule (RNA) comprising L-ribonucleotides (mirror-image with respect to comparable naturally-occurring RNA molecules) having a catalytic activity (a ribozyme) that aminoacylate RNA by using activated amino acids (tRNA charging activity); namely provided herein is an L-flexizyme.

As demonstrated in the Examples section below, charging L-tRNA molecules with D-amino acid residues is more efficient and more consistent using an L-flexizyme.

The L-flexizymes provided herein are having substantially the same sequence as their mirror-image counterparts (D-aaRS ribozymes; D-flexizymes), or exhibit at least 80% sequence identity with respect to the D-flexizyme known in the art. For example, according to some embodiments, the L-flexizyme is having a sequence that exhibits at least 80% identity to 5′-ggaucgaaagauuuccgcauccccgaaaggguacauggcguuaggu-3′(SEQ ID No. 82).

Stemming from the aspect of the L-flexizyme is the use of the L-flexizyme to charge-L-tRNA molecules with pre-activated D-amino acid residues. Thus, according to another aspect of some embodiments of the present invention, there is provided a method charging an L-tRNA with a D-amino acid, which is effected by:
providing an activated D-amino acid;
providing an L-tRNA molecule;
providing an L-flexizyme; and
reacting the L-tRNA, the L-flexizyme and an activated D-amino acid to thereby obtain a D-amino acid-charged L-tRNA molecule.

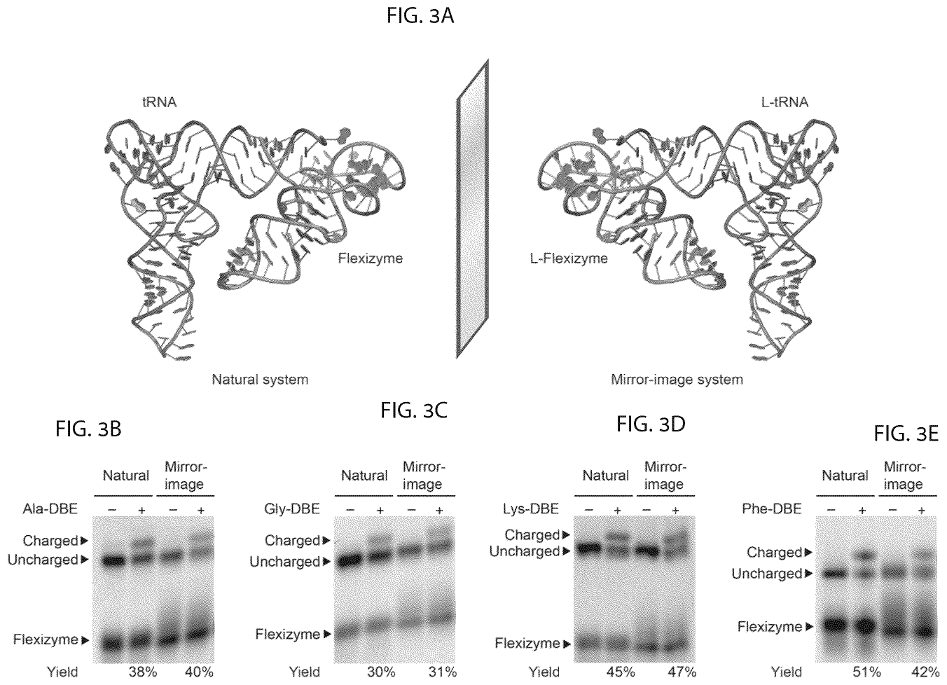

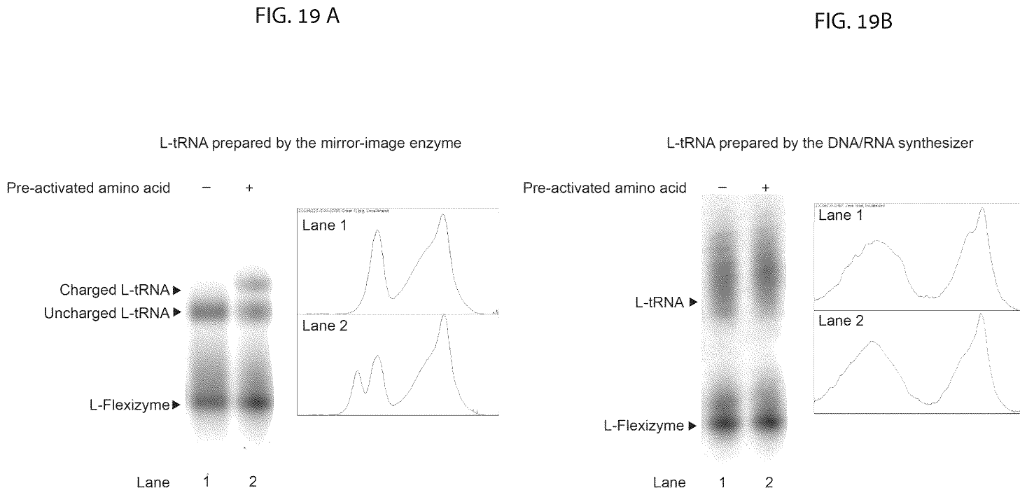

According to some embodiments of the present invention, the L-tRNA molecules are prepared using a D-polymerase, as opposed to a synthesizing machine product. As demonstrated in the Examples section that follows below, the reaction of L-flexizyme with L-tRNA showed a notable improvement in efficiency and fidelity of the aaRS activity (amino-acid charging) reaction when the source of the L-tRNA was enzymatic rather than synthetic (see, discussion regarding FIG. 3A-E and FIG. 19A-B hereinbelow). This advantage can be seen and identified using a PAGE analysis of the reaction mixture of the D-amino acid-charged L-tRNA molecule, that is characterized by a distinct peak for a charged tRNA species and a distinct peak for a uncharged tRNA species, whereas in a reaction using machine-synthesized L-tRNA molecules, the reaction mixture exhibits a continuous large peak, indicating a plurality of intermediates, mismatches and other side-reactions stemming from using L-tRNA of lower quality as a starting material.

A method of using the system:

The use of the system provided herein is different that the use of other known cell-free protein translation systems, and even different that so-far known aaRS-free protein translation systems, at least in the sense that the concentration of the pre-charged tRNA molecules is higher than that used in known systems, and the concentration of Mg⁺² is lower than that used in known systems.

Hence, according to another aspect of some embodiments of the present invention, there is provided a method of producing a protein using the cell-free aaRS-free protein translation system provided herein, which is effected by:
providing the plurality of pre-charged tRNA molecules having no more than the concentration of Mg⁺², as discussed hereinabove (less than half of the concentration of other known cell-free protein translation systems, or less than 100 mM); and
contacting this plurality of charged tRNA molecules with an mRNA molecule encoding the desired protein in the cell-free translation mix, to thereby obtain the protein of interest.

In some embodiments, the method further includes, prior to contacting the pre-charged tRNA preparation with the cell-free translation mix, adjusting the concentration of Mg⁺² to the desired low concentration. The depletion of ions, especially cations, from a system comprising macromolecules, particularly sensitive biomacromolecules, can be effected by any known procedure in the art. For example, Mg⁺² concentration can be lowered, without limitation, by chromatography (e.g., HPLC), alcohol precipitation and followed by washes of the precipitated pellet, ultrafiltration and dialysis; other procedures are also contemplated within the scope of the present invention.

In some embodiments, the method further includes, prior to contacting the pre-charged tRNA preparation with the cell-free translation mix, adjusting the concentration of pre-charged tRNA molecules to the desired high concentration, as this feature is discussed hereinabove. Thus, in some embodiments, the method further includes concentrating the charged tRNA molecules to a concentration that is at least 2-fold greater than the concentration in systems that include aaRS. In some embodiments, this concentration of the pre-charged tRNA molecules is at least 160 μM.

The Examples section that follows below provides a detailed presentation of several embodiments of the system disclosed herein and of the method for using the same to produce proteins in the herein disclosed cell-free, aaRS-free protein translation system.

Products of the disclosed system and method:

As demonstrated by the experimental proof-of-concept presented below, the system and method provided herein can be used to produce proteins that are characterized by exhibiting the structure and function of a comparable protein produced in any in vitro translation system, a cellular system or in any naturally occurring system. The protein produced by the provisions of the present invention can also be mirror-image proteins that have been produced from chirally-inverse elements, including fully active enzymes that catalyze reactions from mirror-image starting materials and produce mirror-image products.

Thus, according to an aspect of some embodiments of the present invention, there is provided a protein produced by the system and/or method provided herein. In some embodiments, the protein is a mirror-image protein (D-protein made substantially form D-amino acid residues).

Exemplary proteins that were demonstrated the use of the system provided herein include chicken lysozyme, Gaussia luciferase, and E. coli TrpRS.

### Libraries:

According to some embodiments, the herein-provided system and method can be used to produce a library of randomized or partially randomized peptides, wherein at least of the peptides comprise at least one unnatural amino acid.

One advantage of the aaRS-free system provided herein is that it requires 21 tRNAs to operate efficiently. There are more than 20 other natural tRNA transcripts available for assigning unnatural amino acids (genetic code reprogramming), and in other protein translation systems, these tRNAs are not usable because aaRS would charge them with natural amino acids. Hence, could the present invention provide the means to translate randomized peptides with multiple unnatural amino acids, while not running into the problem of mis-charged tRNA molecules.

The protein translation system provided herein can by applied to orthogonal ribosome-tRNA pair with compensatory mutations [Terasaka, N., Hayashi, G., Katoh, T., and Suga, H. “An orthogonal ribosome-tRNA pair via engineering of the peptidyl transferase center” Nat. Chem. Biol., 2014, 10, 555-557]. In such orthogonal systems, the orthogonal tRNAs are charged by flexizymes and not chargeable by any aaRS proteins, but suffer from the problem of inefficiencies that are solved by the provision of the present invention. For example, known aaRS-free systems suffers from low yield for translating heptapeptides such as (Fph)-Lys-Tyr-Asp- Lys-Tyr-Asp (SEQ ID No. 125), with yields of about 0.15 μM, and low processivity (7-aa). Under the improved condition afforded by the system according to embodiments of the present invention, the yield is about 2 μM for the same heptapeptide. Moreover, translation up to 334-amino-acid residues, 48 times longer than previously demonstrated, has been demonstrated using the provisions of the present invention. Thus, the improved cell-free/aaRS-free system, according to some embodiments of the present invention, is more adapted for peptide drug discovery due to better yield (more concentrated peptide pools) and longer products (higher sequence diversity).

Non-biologic uses:

In the search for ultra-dense, high fidelity information storing facilities, the present inventors have contemplated the use of the herein-disclosed system and method in the production of proteinous macromolecules having a sequence that can be encoded and decoded using known procedures, yet cannot be degraded by naturally occurring biochemical elements. The protection from biodegradation is afforded by using unnaturally occurring amino-acid residues in the protein.

Moreover, the provisions of the present invention can be used to maximize data density by incorporating unnatural amino acids, which are essentially letters of character-modifiers in the text-analogy.

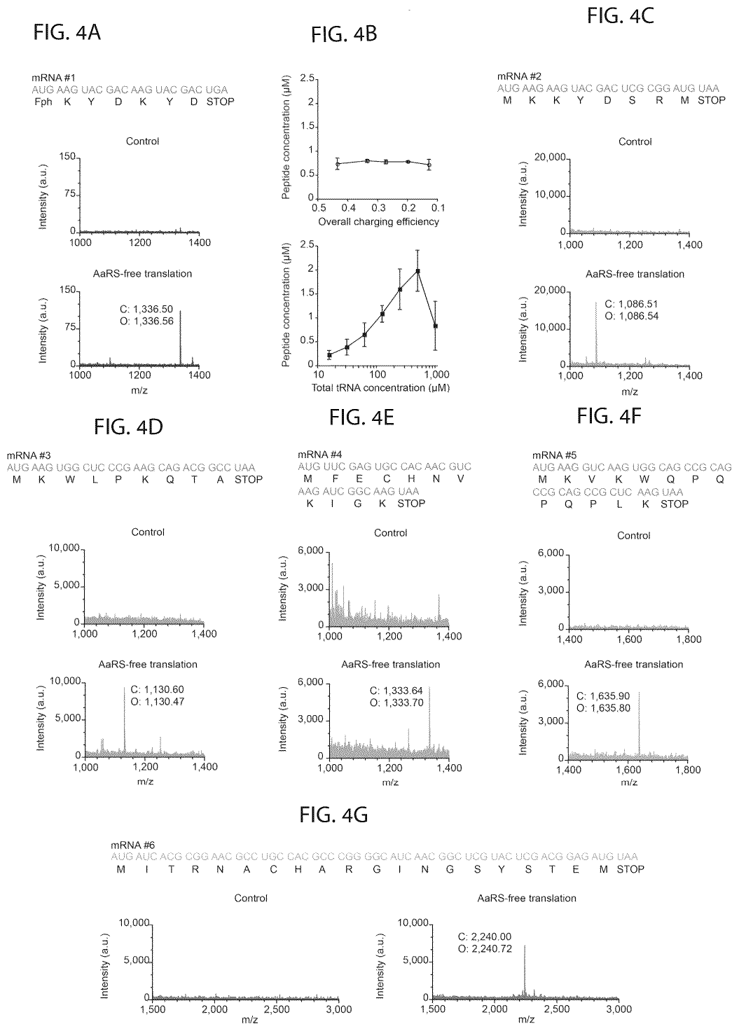

Thus, in some embodiments of the present invention, the protein that is the product of the use of the system provided herein is characterized by having an amino-acid sequence that can be decoded into textual and/or numerical information, and that includes at least some unnaturally occurring amino-acid residues. The realization of this concept requires the translation of peptides of arbitrary sequences. The inventors demonstrated this in FIGS. 4C-G with 20 proteinogenic amino acids.

This concept was realized in the demonstrative proof-of-concept experiment presented in the Examples section that follows below, wherein the inventors encoded a short message “MITRNACHARGINGSYSTEM” (SEQ ID No. 123) into mRNA #6 (see, FIG. 4G) and successfully translated the full-length information-carrying peptide.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

When applied to an original property, or a desired property, or an afforded property of an object or a composition, the term “substantially maintaining”, as used herein, means that the property has not changed by more than 20%, 10% or more than 5% in the processed object or composition.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

It is expected that during the life of a patent maturing from this application many relevant methods for aaRS-free protein translation systems will be developed and the scope of the phrase “aaRS-free protein translation system” is intended to include all such new technologies a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate some embodiments of the invention in a non-limiting fashion.

### Example 1

### Experimental Procedures

### Materials:

Amino acid substrates for flexizyme-charging were prepared as 3,5-dinitrobenzyl esters (DBEs), except for Asn and fluorescein (FAM)-labeled Phe (Fph), which was synthesized as 4-chlorobenzyl thioester (CBT) and cyanomethyl ester (CME), respectively. Amino acid DBEs were either ordered from Nantong Pptide Biotech Ltd (Jiangsu, China) or synthesized in house according to the previously reported method μMurakami, H., Ohta, A., Ashigai, H., and Suga, H. (2006). A highly flexible tRNA acylation method for non-natural polypeptide synthesis. Nat. Methods 3, 357]. All of the amino acid DBE substrates were verified by ¹H-NMR or high-resolution mass spectrometry. Fph-CME was synthesized and verified by high-resolution mass spectrometry as described [Terasaka, N., Hayashi, G., Katoh, T., and Suga, H. (2014). An orthogonal ribosome-tRNA pair via engineering of the peptidyl transferase center. Nat. Chem. Biol. 10, 555-557].

Asn-CBT was synthesized as following: a mixture of 0.5 mmol of Boc-Asn (Trt)-OH, 0.45 mmol of N,N-bis(2-oxo-3-oxazolidiyl)phosphorodiamidic chloride, 1.5 mmol of triethylamine, and 0.5 mmol of 4-chlorobenzyl mercaptan in 5 ml of dichloromethane was stirred for 4 hr at room temperature. The solution was washed with 0.5 μM HCl, 0.5 N NaOH, and brine. The organic layer was dried over anhydrous Na₂SO₄ and concentrated by rotary evaporation. To remove the Boc and trityl protection groups, a 2 ml solution containing 19:1 (v/v) trifluoroacetic acid (TFA)/ddH₂O was added and stirred for 4 hr at room temperature. The solution was neutralized with saturated NaHCO₃, extracted by dichloromethane, and concentrated by rotary evaporation. The crude product was dissolved in methanol and purified by a C18 HPLC column (Inertsil ODS-3, 5 μm, 10×150 mm, GL Sciences, Japan) using a gradient of 30-80% acetonitrile in 0.1% TFA. Fractions containing the product were pooled, lyophilized and verified: ¹H NMR (400 μMHz, DMSO-d₆) δ 8.40 (s, 3H), 7.74 (d, J=9.3 Hz, 1H), 7.44-7.35 (m, 4H), 7.33 (d, J=9.5 Hz, 1H), 4.54-4.46 (m, 1H), 4.25 (d, J=9.4 Hz, 2H), 2.78 (dh, J=14.3, 7.5, 6.4 Hz, 2H). D-DNA oligos were ordered from Genewiz (Jiangsu, China).

RNA oligos and the DNA-RNA chimeric oligo were ordered from Tsingke (Beijing, China). L-DNA oligos and L-flexizyme were synthesized on a H-8 DNA synthesizer (K&A Laborgeraete, Germany) using L-deoxynucleoside and L-2′-t-butyldimethylsilyl (TBDMS) phosphoramidites (Chemgenes, MA, U.S.). Phosphorothioate modification was introduced using Sulfur 42 reagents (Sigma-Aldrich, MO, U.S.). Synthesized L-oligos were cleaved from CPG by concentrated ammonium hydroxide at 65° C. for 2 hr. For the synthesis of L-flexizyme, 2′-TBDMS protecting groups were removed by treatment with 1:1 (v/v) triethylamine trihydrofluoride/DMSO at 65° C. for 2.5 hr.

L-NTPs for mirror-image transcription were prepared from the unprotected L-nucleosides (Chemgenes, MA, U.S.) according to the previously reported method [Caton-Williams, J., Hoxhaj, R., Fiaz, B., and Huang, Z. (2013). Use of a 5′-regioselective phosphitylating reagent for one-pot synthesis of nucleoside 5′-triphosphates from unprotected nucleosides. Curr. Protoc. Nucleic Acid Chem. 52, 1.30.1-1.30.21]. L-DNA oligos and L-NTPs were purified by denaturing PAGE and HPLC, respectively. L-flexizyme was precipitated by ethanol and purified by HPLC.

Genes for AlaRS, AspRS, LysRS, TrpRS and TyrRS were amplified and cloned from E. coli K12 μMG1655 genomic DNA. The gene for Gaussia luciferase was synthesized by Genewiz (Jiangsu, China). Recombinant aaRS proteins and Gaussia luciferase with an N-terminal TEV-cleavable His-tag were expressed and purified from the E. coli strain BL21 as described in the literature [Shimizu, Y., and Ueda, T. (2010). PURE technology. In Cell-Free Protein Production: Methods and Protocols, Y. Endo, K. Takai and T. Ueda, eds. (Totowa, NJ: Humana Press), pp. 11-21]. After purification, the His tag was cleaved by the TEV protease.

Purified chicken egg white lysozyme was purchased from Sigma-Aldrich (MO, U.S.).

In vitro transcription:

The double-stranded DNA (dsDNA) templates for in vitro transcription were prepared by cross-extending two partially overlapped primers (1F and 2R) (2 μM forward primer iF, 3 μM reverse primer 2R, 0.2 mM each dNTP and 5 U EasyTaq (TransGen Biotech, Beijing, China) per 100 l reaction) in a 5-cycle PCR, or by a 25-cycle assembly PCR using four primers (1F, 2R, 3F and 4R) (2 μM each primers iF and 4R, 0.05 μM each primers 2R and 3F, 0.2 mM each dNTP and 5 U EasyTaq per 100 l reaction).

The PCR products were purified by phenol-chloroform followed by ultrafiltration. For the tRNA sequences starting with a 5′ nucleotide other than guanosine, a self-cleaving hammerhead motif is placed upstream of the tRNA sequence [Cui, Z., Stein, V., Tnimov, Z., Mureev, S., and Alexandrov, K. (2015). Semisynthetic tRNA complement mediates in vitro protein synthesis. J. Am. Chem. Soc. 137, 4404-4413]. All primer DNA oligo sequences for assembling dsDNA templates in vitro transcription are listed in Table 1 below.

TABLE 1

| tRNA | Oligo | Sequence | SEQ ID No. |
|---|---|---|---|
| tRNA^{Ala} | 1F | 5′- | 1 |
| | | TTCTAATACGACTCAC | |
| | | TATAGGGGCTATAGCT | |
| | | CAGCTGGGAGAGCGC | |
| | | TTGCATGGCAT-3′ | |
| | 2R | 5′- | 2 |
| | | TGGTGGAGCTAAGCG | |
| | | GGATCGAACCGCTGA | |
| | | CCTCTTGCATGCCATG | |
| | | CAAGCGCTCTCC-3′ | |
| tRNA^{Arg} | 1F | 5′- | 3 |
| | | TTCTAATACGACTCAC | |
| | | TATAGCGCCCGTAGCT | |
| | | CAGCTGGATAGAGCG | |
| | | CTGCCCTCCGGA-3′ | |
| | 2R | 5′- | 4 |
| | | TGGCGCGCCCGACAG | |
| | | GATTCGAACCTGAGA | |
| | | CCTCTGCCTCCGGAG | |
| | | GGCAGCGCTCTATC-3′ | |
| tRNA^{Asn} | 1F | 5′- | 5 |
| | | TTCTAATACGACTCAC | |
| | | TATAGGAGGACTGATG | |
| | | AGTCGGAAACGACGA | |
| | | AACGCGAAAG-3′ | |
| | 2R | 5′- | 6 |
| | | CCGCCGTTCTACCGAC | |
| | | TGAACTACAGAGGAG | |
| | | ACGCTTTCGCGTTTCG | |
| | | TCGTTTCC-3′ | |
| | 3F | 5′- | 7 |
| | | CGGTAGAACGGCGGA | |
| | | CTGTTAATCCGTATGT | |
| | | CACTGGTTCGAGTCC | |
| | | A-3′ | |
| | 4R | 5′- | 8 |
| | | TGGCTCCTCTGACTGG | |
| | | ACTCGAACCAGTGAC | |
| | | ATA-3′ | |
| tRNA^{Asp} | 1F | 5′- | 9 |
| | | TTCTAATACGACTCAC | |
| | | TATAGGAGCGGTAGTT | |
| | | CAGTCGGTTAGAATAC | |
| | | CTGCCTGTCACG-3′ | |
| | 2R | 5′- | 10 |
| | | TGGCGGAACGGACGG | |
| | | GACTCGAACCCGCGA | |
| | | CCCCCTGCGTGACAG | |
| | | GCAGGTATTCTAACC- | |
| | | 3 | |
| tRNA^{Cys} | 1F | 5′- | 11 |
| | | TTCTAATACGACTCAC | |
| | | TATAGGCGCGTTAACA | |
| | | AAGCGGTTATGTAGCG | |
| | | GATTGCAAA-3′ | |
| | 2R | 5′- | 12 |
| | | TGGAGGCGCGTTCCG | |
| | | GAGTCGAACCGGACT | |
| | | AGACGGATTTGCAATC | |
| | | CGCTACATAAC-3′ | |
| tRNA^{fMet} | 1F | 5′- | 13 |
| | | TTCTAATACGACTCAC | |
| | | TATAGGCGGGGTGGA | |
| | | GCAGCCTGGTAGCTC | |
| | | GTCGGGCTCATAA-3′ | |
| | 2R | 5′- | 14 |
| | | TGGTTGCGGGGGCCG | |
| | | GATTTGAACCGACGAT | |
| | | CTTCGGGTTATGAGCC | |
| | | CGACGAGCTACC-3′ | |
| tRNA^{Gln} | 1F | 5′- | 15 |
| | | TTCTAATACGACTCAC | |
| | | TATAGGCCCCACTGAT | |
| | | G-3′ | |
| | 2R | 5′- | 16 |
| | | CAGACGCTTTCGCGTT | |
| | | TCGTCGTTTCCGACTC | |
| | | ATCAGTGGGGCCTATA | |
| | | GT-3′ | |
| | 3F | 5′- | 17 |
| | | ACGCGAAAGCGTCTG | |
| | | GGGTATCGCCAAGCG | |
| | | GTAAGGCACCGGATTC | |
| | | TGATTCCGGCATT-3′ | |
| | 4R | 5′- | 18 |
| | | TGGCTGGGGTACGAG | |
| | | GATTCGAACCTCGGA | |
| | | ATGCCGGAATCAGAAT | |
| | | CCGGTGCCTTA-3′ | |
| tRNA^{Glu} | 1F | 5′- | 19 |
| | | TAATACGACTCACTAT | |
| | | AGGGACCTGATGAGT | |
| | | CGGAAACG-3′ | |
| | 2R | 5′- | 20 |
| | | GACCTGATGAGTCGG | |
| | | AAACGACGAAACGCG | |
| | | AAAGCGTCGTCCCCTT | |
| | | CGTCTAGAGGCCCA-3′ | |
| | 3F | 5′- | 21 |
| | | ATTCGAACCCCTGTTA | |
| | | CCGCCGTGAGAGGGC | |
| | | GGTGTCCTGGGCCTCT | |
| | | AGACGAAGGG-3′ | |
| | 4R | 5′- | 22 |
| | | TGGCGTCCCCTAGGG | |
| | | GATTCGAACCCCTGTT | |
| | | ACCG-3′ | |
| tRNA^{Gly} | 1F | 5′- | 23 |
| | | TTCTAATACGACTCAC | |
| | | TATAGCGGGAATAGCT | |
| | | CAGTTGGTAGAGCAC | |
| | | GACCTTGCCAA-3′ | |
| | 2R | 5′- | 24 |
| | | TGGAGCGGGAAACGA | |
| | | GACTCGAACTCGCGA | |
| | | CCCCGACCTTGGCAA | |
| | | GGTCGTGCTCTAC-3′ | |
| tRNA^{His} | 1F | 5′- | 25 |
| | | TTCTAATACGACTCAC | |
| | | TATAGTGGCTATAGCT | |
| | | CAGTTGGTAGAGCCCT | |
| | | GGATTGTGAT-3′ | |
| | 2R | 5′- | 26 |
| | | TGGGGTGGCTAATGG | |
| | | GATTCGAACCCACGA | |
| | | CAACTGGAATCACAAT | |
| | | CCAGGGCTCTAC-3′ | |
| tRNA^{Ile} | 1F | 5′- | 27 |
| | | TTCTAATACGACTCAC | |
| | | TATAGGGCTTGTAGCT | |
| | | CAGGTGGTTAGAGCG | |
| | | CACCCCTGATAA-3′ | |
| | 2R | 5′- | 28 |
| | | TGGTGGGCCTGAGTG | |
| | | GACTTGAACCACCGA | |
| | | CCTCACCCTTATCAGG | |
| | | GGTGCGCTCTAAC-3′ | |
| tRNA^{Leu} | 1F | 5′- | 29 |
| | | TTCTAATACGACTCAC | |
| | | TATAGCCGAGGTGGTG | |
| | | GAAT-3′ | |
| | 2R | 5′- | 30 |
| | | ACCTCAAGGTAGCGT | |
| | | GTCTACCAATTCCACC | |
| | | ACCTCGGC-3′ | |
| | 3F | 5′- | 31 |
| | | ACACGCTACCTTGAG | |
| | | GTGGTAGTGCCCAATA | |
| | | GGGCTTACGGGTT-3′ | |
| | 4R | 5′- | 32 |
| | | TGGTACCGAGGACGG | |
| | | GACTTGAACCCGTAA | |
| | | GCCCTATTGGGCA-3′ | |
| tRNA^{Lys} | 1F | 5′- | 33 |
| | | TTCTAATACGACTCAC | |
| | | TATAGGGTCGTTAGCT | |
| | | CAGTTGGTAGAGCAG | |
| | | TTGACTCTTAA-3′ | |
| | 2R | 5′- | 34 |
| | | TGGTGGGTCGTGCAG | |
| | | GATTCGAACCTGCGA | |
| | | CCAATTGATTAAGAGT | |
| | | CAACTGCTCTAC-3′ | |
| tRNA^{Met} | 1F | 5′- | 35 |
| | | TTCTAATACGACTCAC | |
| | | TATAGGCTACGTAGCT | |
| | | CAGTTGGTTAGAGCA | |
| | | CATCACTCATA-3′ | |
| | 2R | 5′- | 36 |
| | | TGGTGGCTACGACGG | |
| | | GATTCGAACCTGTGAC | |
| | | CCCATCATTATGAGTG | |
| | | ATGTGCTCTAA-3′ | |
| tRNA^{Phe} | 1F | 5′- | 37 |
| | | TTCTAATACGACTCAC | |
| | | TATAGCCCGGATAGCT | |
| | | CAGTCGGTAGAGCAG | |
| | | GGGATTGAAAA-3′ | |
| | 2R | 5′- | 38 |
| | | TGGTGCCCGGACTCG | |
| | | GAATCGAACCAAGGA | |
| | | CACGGGGATTTTCAAT | |
| | | CCCCTGCTCTAC-3′ | |
| tRNA^{Pro} | 1F | 5′- | 39 |
| | | TTCTAATACGACTCAC | |
| | | TATAGGCACCGCTGAT | |
| | | GAGTCGGAAACGACG | |
| | | AAACGCGA-3′ | |
| | 2R | 5′- | 40 |
| | | CCAGGCTGCGCCAAT | |
| | | CACCGGACGCTTTCG | |
| | | CGTTTCGTCGTTTCCG | |
| | | ACTCAT-3′ | |
| | 3F | 5′- | 41 |
| | | GGCGCAGCCTGGTAG | |
| | | CGCACTTCGTTCGGG | |
| | | ACGAAGGGGTCGGAG | |
| | | GTTCGAAT-3′ | |
| | 4R | 5′- | 42 |
| | | TGGTCGGTGATAGAG | |
| | | GATTCGAACCTCCGAC | |
| | | CCCTT-3′ | |
| tRNA^{Ser} | 1F | 5′- | 43 |
| | | TTCTAATACGACTCAC | |
| | | TATAGGAGAGATGCCG | |
| | | GAGCGGCTG-3′ | |
| | 2R | 5′- | 44 |
| | | GCCCCTACTCCGGTTT | |
| | | TCGAGACCGGTCCGT | |
| | | TCAGCCGCTCCGGCAT | |
| | | -3′ | |
| | 3F | 5′- | 45 |
| | | ACCGGAGTAGGGGCA | |
| | | ACTCTACCGGGGGTTC | |
| | | AAATCCCCCTCTCTCC | |
| | | GCCA-3′ | |
| | 4R | 5′- | 46 |
| | | TGGCGGAGAGAGGGG | |
| | | GATT-3′ | |
| tRNA^{Thr} | 1F | 5′- | 47 |
| | | TTCTAATACGACTCAC | |
| | | TATAGCCGATATAGCT | |
| | | CAGTTGGTAGAGCAG | |
| | | CGCATTCGTAA-3′ | |
| | 2R | 5′- | 48 |
| | | TGGTGCCGATAATAGG | |
| | | AGTCGAACCTACGAC | |
| | | CTTCGCATTACGAATG | |
| | | CGCTGCTCTAC-3′ | |
| tRNA^{Trp} | 1F | 5′- | 49 |
| | | TTCTAATACGACTCAC | |
| | | TATAGGCCCCTCTGAT- | |
| | | 3′ | |
| | 2R | 5′- | 50 |
| | | CGCCCCTGACGCTTTC | |
| | | GCGTTTCGTCGTTTCC | |
| | | GACTCATCAGAGGGG | |
| | | CCTATAGTGA-3′ | |
| | 3F | 5′- | 51 |
| | | GCGTCAGGGGCGTAG | |
| | | TTCAATTGGTAGAGCA | |
| | | CCGGTCTCCAAAACC | |
| | | GGGTGT-3′ | |
| | 4R | 5′- | 52 |
| | | TGGCAGGGGCGGAGA | |
| | | GACTCGAACTCCCAA | |
| | | CACCCGGTTTTGGAG | |
| | | ACC-3′ | |
| tRNA^{Tyr} | 1F | 5′- | 53 |
| | | TTCTAATACGACTCAC | |
| | | TATAGGTGGGGTTCCC | |
| | | GAGCGGCCAA-3′ | |
| | 2R | 5′- | 54 |
| | | CGGCAGATTTACAGTC | |
| | | TGCTCCCTTTGGCCGC | |
| | | TCGGG-3′ | |
| | 3F | 5′- | 55 |
| | | GCAGACTGTAAATCTG | |
| | | CCGTCATCGACTTCGA | |
| | | AGGTTCGAAT-3′ | |
| | 4R | 5′- | 56 |
| | | TGGTGGTGGGGGAAG | |
| | | GATTCGAACCTTCGAA | |
| | | GTCGATG-3′ | |
| tRNA^{Val} | 1F | 5′- | 57 |
| | | TTCTAATACGACTCAC | |
| | | TATAGCGTCCGTAGCT | |
| | | CAGTTGGTTAGAGCA | |
| | | CCACCTTGACAT-3′ | |
| | 2R | 5′- | 58 |
| | | TGGTGCGTCCGAGTG | |
| | | GACTCGAACCACCGA | |
| | | CCCCCACCATGTCAAG | |
| | | GTGGTGCTCTAAC-3′ | |
| dinitro- | 1F | 5′- | 59 |
| flexizyme | | TTCTAATACGACTCAC | |
| | | TATAGGATCGAAAGAT | |
| | | TTCCGCATCCC-3′ | |
| | 2R | 5′- | 60 |
| | | ACCTAACGCCATGTAC | |
| | | CCTTTCGGGGATGCGG | |
| | | AAATCTTTCGA-3′ | |
| enhanced | 1F | 5′- | 61 |
| flexizyme | | TTCTAATACGACTCAC | |
| | | TATAGGATCGAAAGAT | |
| | | TTCCGCGGCCC-3′ | |
| | 2R | 5′- | 62 |
| | | ACCTAACGCTAATCCC | |
| | | CTTTCGGGGCCGCGG | |
| | | AAATCTT-3′ | |

The 1 ml transcription reaction systems contained 20 tg of purified dsDNA template, 2 mM each NTP, 0.1 mg/mi of T7 RNA polymerase, 400 U of RiboLock RNase inhibitor (Thermo Fisher Scientific, MA, U.S.) in 1 × transcription buffer containing 25 mM MgCl2, 40 mM Tris-HCl pH 8.0, 2 mM DTT, and 1 mM spermnidine. Transcription reactions were incubated at 37° C. for 2 hr, treated with 10 μ of DNase I (New England Biolabs, MA, U.S.), and incubated for another 0.5 hr, before being quenched by the addition of 60 tl of 0.5 μM EDTA, followed by ethanol precipitation. The transcribed RNAs were gel-purified using the “crush and soak” method,!desalted and concentrated by ultrafiltration. Both 5′-triphosphate (tRNA sequences starting with G) and 5′-hydroxyl-terminated tRNAs (tRNA sequences starting with A/U/C) were prepared using this method, which were previously shown to be functionally equivalent to the physiological 5′-monophosphate-terminated tRNAs with tRNA^{H}is as an exception [Cui, Z., Stein, V., Tnimov, Z., Mureev, S., and Alexandrov, K. (2015). Semisynthetic tRNA complement mediates in vitro protein synthesis. J. Am. Chem. Soc. 137, 4404-4413]. Thus, two versions of tRNA^{H}is were prepared: 5′-triphosphate-terminated tRNA^{H}is was used in all flexizyme-charging assays; 5′-monophosphate-terminated tRNA^{H}is carrying an additional G at position -1 was prepared by the previously reported method and used in controls that required aaRS activity.

Flexizyme-catalyzed tRNA-aminoacylation:

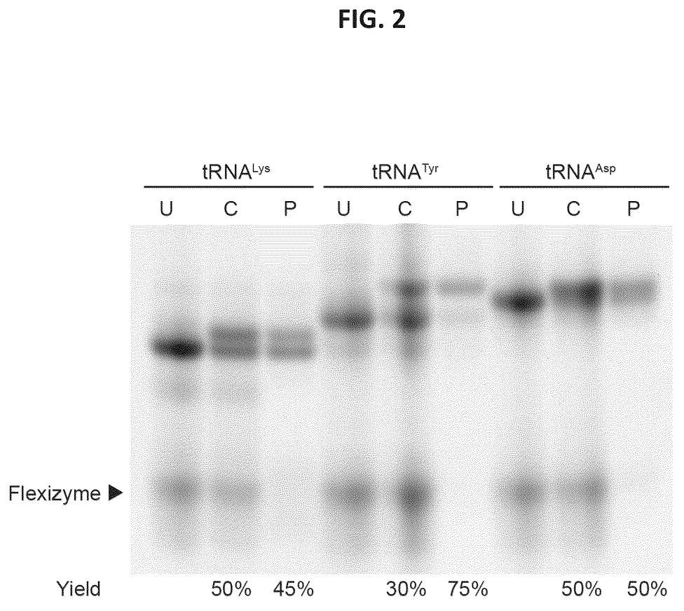

The procedures for performing flexizyme-catalyzed tRNA charging are adapted from the previously reported method [Goto, Y., Katoh, T., and Suga, H. (2011). Flexizymes for genetic code reprogramming. Nat. Protoc. 6, 779-790]: 20 μM tRNA was mixed with 30 μM dinitro-flexizyme in the presence of 1×folding buffer containing 50 mM HEPES-KOH and 100 mM KCl at pH 7.5. The mix was heated at 95° C. for 2 min, cooled to 25° C. for 10 min, and followed by the addition of 100 mM MgCl₂. The mix was incubated for 10 min at room temperature and 3 min on a refrigerated metal block. 5 mM DBE substrate was added to the system on a cold metal block to initiate the charging reaction. The reaction was incubated at 4° C. for 6 hr, and quenched with 2×volumes of 0.6 μM NaOAc at pH 5.2 and precipitated by ethanol. Adjustments to the general procedure were made for the following amino acids: for Ile-DBE and Val-DBE, the refolding buffer was changed to 50 mM bicine-KOH at pH 9.0; for Met-DBE and Cys-DBE, 5 mM of DTT was supplemented with the substrate; for Pro-DBE, substrate concentration was increased from 5 to 40 mM; for Fph-CME, 30 μM of enhanced flexizyme was used, the Fph-CME substrate concentration was reduced from 5 to 1 mM, and the MgCl₂ concentration was increased from 100 to 400 mM; for Asn-CBT, the substrate concentration was increased from 5 to 25 mM; for Trp-DBE, an additional 20% DMSO was added. Charging yields were determined by acid PAGE analysis: the precipitated charging reaction was dissolved in a loading buffer containing 93% formamide, 100 mM NaOAc at pH 5.2, 10 mM EDTA, and trace amounts of bromophenol blue. Acid gel was prepared by 8% acrylamide, 100 mM NaOAc at pH 5.2, and 7 μM urea. The gel was run inside a 4° C. refrigerator with an aluminum cooling plate for 16 hr with 100 mM NaOAc as running buffer at pH 5.2. The gel was stained by SYBR-Green II (Thermo Fisher Scientific, MA, U.S.), scanned by Typhoon FLA 9500 operated under Cy2 mode, and analyzed by the software package ImageJ [Schneider, C. A.; Rasband, W. S. & Eliceiri, K. W. (2012), “NIH Image to ImageJ: 25 years of image analysis”, Nature methods 9(7): 671-675, PMID 22930834]. Peak area was integrated for calculating the yield of charged tRNAs with the exception of tRNAAs. Peak height was used for estimating the yield of Asp-tRNA^{Asp}, which migrated very closely with the uncharged tRNA^{Asp} (see, FIG. 2), and the estimate of Asp-tRNA^{Asp} charging yield (-50%) was consistent with the previously reported results μMurakami, H., Ohta, A., Ashigai, H., and Suga, H. (2006). A highly flexible tRNA acylation method for non-natural polypeptide synthesis. Nat.

### Methods 3, 357].

FIG. 2. Presents an acid PAGE analysis of tRNA charging yields before and after HPLC purification, wherein “U” represents uncharged tRNA, “C” represents crude charged tRNA, “P” represents purified charged tRNA, whereas the tRNA charging yields were determined by software package ImageJ using the integrated peak area of charged tRNAs relative to the total tRNAs.

AaRS-free translation of multiple short peptides:

The dsDNA templates for aaRS-free peptide translation were prepared by 25-cycle assembly PCR using the primers listed in Table 2.

TABLE 2

| mRNA | Oligo | Sequence | SEQ ID No. |
|---|---|---|---|
| | Universal_F | 5′- | 63 |
| | | GGCGTAATACGACT | |
| | | CACTATAGGGTTAA | |
| | | CTTTAAGAAGGAGA | |
| | | TATACCA-3′ | |
| mRNA #1 | 7mer_F | 5′- | 64 |
| | | TTAACTTTAAGAAG | |
| | | GAGATATACCAATG | |
| | | AAGTACGACAAG-3′ | |
| | 7mer_R | 5′- | 65 |
| | | CGAAGCTCAGTCGT | |
| | | ACTTGTCGTACTTC | |
| | | ATTGGTATAT-3′ | |
| mRNA #2 | 8mer_F | 5′- | 66 |
| | | TTAACTTTAAGAAG | |
| | | GAGATATACCAATG | |
| | | AAGAAGTACGACT- | |
| | | 3′ | |
| | 8mer_R | 5′- | 67 |
| | | CGAAGCTTACATCC | |
| | | GCGAGTCGTACTTC | |
| | | TTCATTGGTATATC | |
| | | T-3′ | |
| mRNA #3 | 9mer_F | 5′- | 68 |
| | | TTAACTTTAAGAAG | |
| | | GAGATATACCAATG | |
| | | AAGTGGCTCCCGA | |
| | | A-3′ | |
| | 9mer_R | 5′- | 69 |
| | | CGAAGCTTAGGCCG | |
| | | TCTGCTTCGGGAGC | |
| | | CACTTCATTGGTAT | |
| | | A-3′ | |
| mRNA #4 | 11mer F | 5′- | 70 |
| | | TTAACTTTAAGAAG | |
| | | GAGATATACCAATG | |
| | | TTCGAGTGCCACAA | |
| | | CG-3′ | |
| | 11mer_R | 5′- | 71 |
| | | CGAAGCTTACTTGC | |
| | | CGATCTTGACGTTG | |
| | | TGGCACTCGAACAT | |
| | | T-3′ | |
| mRNA #5 | 13mer_F | 5′- | 72 |
| | | TTAACTTTAAGAAG | |
| | | GAGATATACCAATG | |
| | | AAGGTCAAGTGGC | |
| | | AGCC-3′ | |
| | 13mer_R | 5′- | 73 |
| | | CGAAGCTTACTTGA | |
| | | GCGGCTGCGGCTGC | |
| | | GGCTGCCACTTGAC | |
| | | CTT-3′ | |
| mRNA #6 | 20mer_F | 5′- | 74 |
| | | TTAACTTTAAGAAG | |
| | | GAGATATACCAATG | |
| | | ATCACGCGGAACGC | |
| | | CTGCCACGCCCGG | |
| | | G-3′ | |
| | 20mer_R | 5′- | 75 |
| | | CGAAGCTTACATCT | |
| | | CCGTCGAGTACGAG | |
| | | CCGTTGATGCCCCG | |
| | | GGCGTGGCAGG-3′ | |

All DNA templates were purified by 10% denaturing PAGE. To translate mRNA #1, the reaction mix was adjusted so that each codon would be decoded by a cognate flexizyme-charged tRNA ranging from 1.25-80 tM, which correspond to 16-1000 tM total tRNAs. HPLC purification of flexizyme-charged tRNAs was performed as described in the reference [lZhang, J., and Ferre-D′Amare, Adrian R. (2014). Direct evaluation of tRNA aminoacylation status by the T-Box riboswitch using tRNA-mRNA stacking and steric readout. Mol. Cell 55, 148-155]. The charged tRNAs were stored and remained stable as dried pellets at −80° C. for up to 3 days, as determined by acid PAGE analysis. The charged tRNA pellets were dissolved in 0.5×translation volume of 1 mM pH 5.2 NaOAc. Successive dilution was performed to generate tRNAs that were twice as concentrated as indicated in Table 3. Table 3 presents the total tRNA concentrations used for aaRS-free translation.

TABLE 3

| | Total | Overall | Translation | |
|---|---|---|---|---|
| | tRNA | charging | reaction | |
| | concentration | yield | volume | Detection |
| Template | (μM) | (%) | (μl) | method |
| mRNA #1 | 80 | 43.5 | 20 | MALDI-TOF-MS |
| | 161-541 | 12.9-43.5 | 5 | Tricine- |
| | 16-1003 | 55.8 | 5 | SDS-PAGE |
| | 20-644 | 43.5 | 5 | |
| | 161 | 43.5 | 5 | |
| | 161-536 | 13.1-43.5 | 5 | |
| | 161-536 | 13.1-43.5 | 5 | |
| | 21-680 | N/A | 5 | |
| mRNA #2 | 170 | 47.1 | 20 | MALDI-TOF-MS |
| mRNA #3 | 272 | 33.1 | 20 | |
| mRNA #4 | 263 | 41.8 | 20 | |
| mRNA #5 | 414 | 31.4 | 20 | |
| mRNA #6 | 263 | 38.0 | 10 | |
| | 527 | 38.0 | 20 | |
| Chicken | 328 | 39.6 | 20 | SDS-PAGE |
| lysozyme | | | 50 | Lysozyme |
| | | | | assay |
| Gaussia | 428 | 39.5 | 20 | SDS-PAGE |
| luciferase | | | 20 | Luciferase |
| | | | | assay |
| E. coli | 169 | 41.8 | 20 | SDS-PAGE |
| TrpRS | | | 20 | LC-MS/MS |
| | | | 20 | Charging |
| | | | | assay |

An equal volume of 2×aaRS-free translation mix that had been preincubated at 37° C. for 5 min was mixed with the tRNAs to initiate the translation reaction. All translation reactions were incubated at 37° C. for 2 hr. Translation was terminated by placing the reaction mix at −20° C., before analysis by 17% or 20% Tricine-SDS-PAGE to determine the translation yield of Fph-labeled peptides. Between 0.125-4 tM of peptide standards (Fph-K-Y-D-K-Y-D (SEQ ID No. 125), custom synthesized by Genscript, Jiangsu, China) were also loaded for calibration. The titration of uncharged tRNAs was performed with the molar ratio of tRNA^{fMet}: tRNA^{LYs}: tRNA^{Tyr}: tRNA^{Asp}=1: 2.5: 2.5: 2.5, mixed with flexizyme-charged tRNAs, incubated briefly at room temperature before being added to the aaRS-free translation system. The final concentrations of the uncharged tRNAs were between 90-470 μM. Flexizyme titration was performed by mixing dinitro-flexizyme with flexizyme-charged tRNAs in 1 mM NaOAc, before being mixed with equal volume of 2×aaRS-free translation mix to initiate the translation reaction. The final concentrations of flexizyme were between 240-520 μM. Titration of folded flexizyme and tRNA complex was performed in a system containing 50 μM of dinitro-flexizyme, 6 μM of tRNA^{fMet}, 15 M each tRNA^{LYs}, tRNA^{T}yr, and tRNA^{Asp}, heated to 95° C. for 2 min in the presence of 50 mM HEPES-KOH at pH 7.5 and 100 mM KCl. The mix was slowly cooled to 25° C., followed by the addition of either 100 mM MgCl₂ or 10 mM MgCl₂, incubated at room temperature for 10 min. The mix was then precipitated by ethanol, washed twice with 70% ethanol and air-dried. The pellet was dissolved in ddH₂O in a small volume and serial-diluted to reach 2×concentrations of flexizyme-tRNA complex at 750, 375, and 188 μM, respectively, before being added to an equal volume of aaRS-free translation system, along with the control containing only ddH₂O.

### MALDI-TOF MS:

MALDI-TOF MS was used to analyze the aaRS-free translation of mRNAs #2 to #6. To reduce peptide drop-off, the scale of each charging reaction was adjusted according to the codon abundance in each gene, so that each codon would match with an equal concentration of flexizyme-charged tRNAs (10 μM per codon for mRNA #2 to #5, 5 μM for mRNA #6). The controls with uncharged tRNAs contained 30 μM (each) of tRNA^{Asn}, tRNA^{Glu}, tRNA^{LYs}, tRNA^{Ile}, and 5 μM (each) of other tRNAs, as well as 100 μM of each amino acid. The charging reactions were quenched, precipitated, and washed once by 70% ethanol. The washed pellets of different flexizyme-charged tRNAs were dissolved in 0.3 μM NaOAc, mixed, precipitated again by ethanol, stored at −80° C., and washed once with 70% ethanol before use. AaRS-free translation was performed by mixing the flexizyme-charged tRNAs with an equal volume of 2×aaRS-free translation mix. After translation for 2 hr at 37° C., TFA was added to the translation system to lower the pH to <4, and the sample was briefly centrifuged with the supernatant desalted using a C18 spin column (Thermo Fisher Scientific, MA, U.S.). After elution, the sample volume was reduced to ˜2-3 μl by a centrifugal vacuum concentrator (Eppendorf, Germany), of which 0.5 l was used for MALDI-TOF analysis under positive reflectron mode (Applied Biosystems 4800 plus MALDI TOF/TOF analyzer, CA, U.S.). The control experiments with uncharged tRNAs and free amino acids (100 μM for each amino acid species) were performed, desalted, and analyzed by MALDI-TOF MS in parallel. The concentrations of uncharged tRNAs used in the control experiments were: 30 μM (each) for tRNA^{Asn}, tRNA^{Glu}, tRNA^{LYs}, tRNA^{I}Ie, and 5 μM (each) for the other tRNAs.

Protein identification by LC-MS/MS:

A volume of 20 μl of crude aaRS-free translated N-terminal FAM-labeled E. coli TrpRS was separated by 15% SDS-PAGE, and silver-stained by the ProteoSilver silver stain kit (Sigma-Aldrich, MO, U.S.). The protein band(s) between EF-Tu (43 kDa) and MTF (34 kDa) were excised from the gel, reduced by 5 mM of dithiotreitol, and alkylated by 11 mM iodoacetamide. In-gel digestion was carried out with sequencing grade trypsin in 50 mM ammonium bicarbonate at 37° C. overnight. The peptides were extracted twice with 0.1% TFA in 50% acetonitrile aqueous solution for 30 min. The extracts were then concentrated by a centrifugal vacuum concentrator. Tryptic peptides were dissolved in 20 l 0.1% TFA and analyzed by LC-MS/MS. The control experiments with uncharged tRNAs and free amino acids using the concentrations as described above were performed and analyzed in parallel.

Enzymatic transcription of mirror-image tRNAs:

The synthesis and folding of D-polymerase D-Dpo4-5m-Y12S for mirror-image transcription was previously reported [Jiang, W., Zhang, B., Fan, C., Wang, M., Wang, J., Deng, Q., Liu, X., Chen, J., Zheng, J., Liu, L., et al. (2017). Mirror-image polymerase chain reaction. Cell Discov. 3, 17037; Xu, W., Jiang, W., Wang, J., Yu, L., Chen, J., Liu, X., Liu, L., and Zhu, T.F. (2017). Total chemical synthesis of a thermostable enzyme capable of polymerase chain reaction. Cell Discov. 3, 17008; Wang, M., Jiang, W., Liu, X., Wang, J., Zhang, B., Fan, C., Liu, L., Pena-Alcantara, G., Ling, J.-J., Chen, J., et al. (2019). Mirror-image gene transcription and reverse transcription. Chem 5, 848-857]. All L-DNA primer, template sequences, and L-nucleic acid oligo sequences are listed in Table 4, wherein “*” denotes phosphorothioate modification, UPPER-case letters denote L-DNA nucleotides, and lower-case letters denote L-RNA nucleotides.

TABLE 4

| | | SEQ |
|---|---|---|
| Oligo | Sequence | ID No. |
| L-tRNA ^{Ala} | 5′- | 76 |
| template | TGGTGGAGCTAAGCGGGATCGAAC | |
| | CGCTGACCTCTTGCATGCCATGCAA | |
| | GCGCTCTCCCAGCTGAGCTATAGCC | |
| | CCTATAGTGAGTCGTATTAGAACCG | |
| | -3′ | |
| L-tRNA ^{Gly} | 5′- | 77 |
| template | TGGAGCGGGAAACGAGACTCGAA | |
| | CTCGCGACCCCGACCTTGGCAAGG | |
| | TCGTGCTCTACCAACTGAGCTATTC | |
| | CCGCTATAGTGAGTCGTATTAGAAC | |
| | CG-3′ | |
| L-tRNA ^{Lys} | 5′- | 78 |
| template | TGGTGGGTCGTGCAGGATTCGAAC | |
| | CTGCGACCAATTGATTAAGAGTCA | |
| | ACTGCTCTACCAACTGAGCTAACG | |
| | ACCCTATAGTGAGTCGTATTAGAAC | |
| | CG-3 | |
| L-tRNA ^{Phe} | 5′- | 79 |
| template | TGGTGCCCGGACTCGGAATCGAAC | |
| | CAAGGACACGGGGATTTTCAATCC | |
| | CCTGCTCTACCGACTGAGCTATCCG | |
| | GGCTATAGTGAGTCGTATTAGAACC | |
| | G-3′ | |
| L-universal | 5′- | 80 |
| primer | CGGTTCTAATACGACTCACTATA*g- | |
| | 3′ | |
| 5′-FAM labeled | 5′-FAM- | 81 |
| L-universal | CGGTTCTAATACGACTCACTATA-3′ | |
| primer | 5′- | 82 |
| L-flexizyme | ggaucgaaagauuuccgcauccccga | |
| | aaggguacauggcguuaggu-3′ | |

Mirror-image transcription was performed with a 24-nt primer-binding site tethered to the 3′-end of the mirror-image single-stranded DNA (L-ssDNA) template to facilitate the RNA purification by PAGE through different product lengths (so that the L-RNA transcripts would be 23-nt shorter than the 99-nt L-ssDNA templates, which can be separated on 12% denaturing PAGE in 7M urea). An L-primer was designed to include a phosphorothioate-modified L-RNA nucleotide at the 3′-end. The enzymatic transcription of mirror-image tRNA^{A}La, tRNA^{GY}, tRNA^{LYs}, and tRNA^{Ph}ie was performed. After mirror-image transcription, the L-primer was efficiently cleaved at the phosphorothioate site by 100 μM 12 in ethanol at 70° C. for 10 min, producing mature mirror-image tRNA transcripts. For the RNase A digestion, 0.4 μM of D- or L- tRNA^{A} l^{a} was mixed with 4 μM of RNase A, incubated at 37° C. for 15 min, and analyzed by 10% denaturing PAGE in 7 μM urea. For aaRS-catalyzed aminoacylation, 5 μM of D- or LAtRNA^{Ala} was mixed with 1 μM of AlaRS in the presence of 10 mM ATP and 100 μM L-or D-alanine, incubated at 37° C. for 1 hr, and analyzed by 8% acid PAGE.

Mirror-image tRNA charging:

Mirror-image tRNA charging was performed using the same aminoacylation method described above, except that L-tRNA and L-flexizyme concentrations were scaled down to 2 μM and 10 aM, respectively. The mirror-image tRNAs were transcribed by the synthetic D-Dpo4-5m-Y12S polymerase, and the natural tRNAs were synthesized either by a recombinant Y12S mutant of Dpo4 (L-Dpo4-5m-Y12S) (SEQ ID No. 126) (tRNA^{Ala}, tRNA^{G}lY and tRNA^{Lys}) or by the T7 RNA polymerase (tRNA^{Phe}). The tRNA charging yields were determined by the software package ImageJ using the integrated peak area of charged tRNAs relative to the total tRNAs.

### Example 2

### Flexizyme-Catalyzed tRNA-Aminoacylation

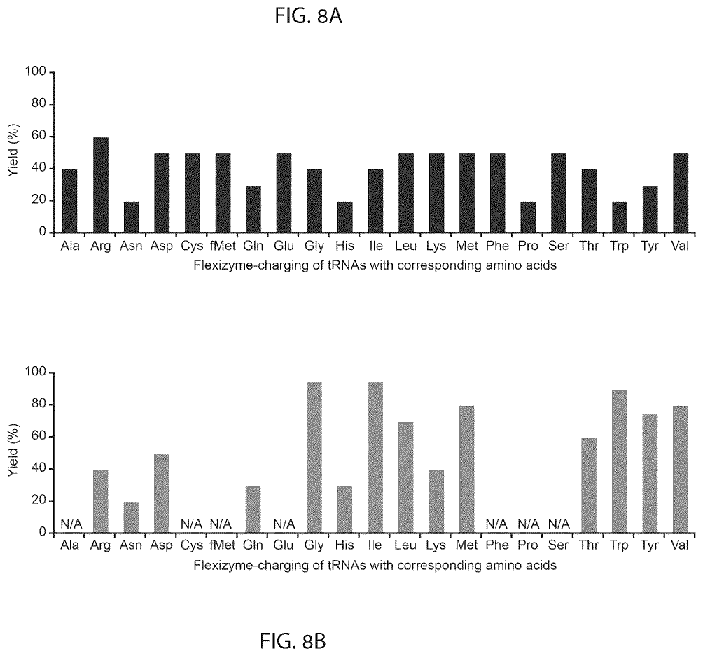

The 21 tRNAs were individually charged with cognate amino acids by the 46-nt dinitro-flexizyme or the 45-nt enhanced flexizyme. The charging reactions were quenched by 0.3 μM NaOAc and precipitated. The pellets were purified by either 70% ethanol wash or a Shimadzu Prominence HPLC system (Japan) as appropriate (see, FIG. 8A and FIG. 8B).

FIGS. 3A-E present concept and results of flexizyme charging of tRNAs en route to an aaRS-free charging of mirror-image tRNAs, according to some embodiments of the present invention, showing D-tRNA charging catalyzed by D-flexizyme, and its mirror-image version, mirror-image tRNA charging catalyzed by L-flexizyme (PDB sources: 1EHZ (tRNA), 3CUL (flexizyme)(FIG. 3A), L-flexizyme charging of D-alanine onto enzymatically transcribed mirror-image tRNAAla, with the natural-chirality counterparts shown for comparison (FIG. 3B), L-flexizyme charging of glycine onto enzymatically transcribed mirror-image tRNAGly, with the natural-chirality counterparts shown for comparison (FIG. 3C) L-flexizyme charging of D-lysine onto enzymatically transcribed mirror-image tRNALys, with the natural-chirality counterparts shown for comparison (FIG. 3D), L-flexizyme charging of D-phenylalanine onto enzymatically transcribed mirror-image tRNAPhe, with the natural-chirality counterparts shown for comparison (FIG. 3E), whereas the tRNA charging yields were determined using software package ImageJ using the integrated peak area of charged tRNAs relative to the total tRNAs.

Symmetry Shield RP18 columns (3.5 m, 4.6×150 mm and 3.5 m, 4.6×100 mm) (Waters Corp, MA, U.S.) were used for HPLC purification, with elution conditions adapted from the literature [Zhang, J., and Ferr6-D′Amard, Adrian R. (2014). Direct evaluation of tRNA aminoacylation status by the T-Box riboswitch using tRNA-mRNA stacking and steric readout. Mol. Cell 55, 148-155]. The fractions containing flexizyme-charged tRNAs were precipitated, dissolved in 10 mM NaOAc at pH 5.2, and the concentration was measured by the Nanodrop spectrophotometer (Thermo Fisher Scientific, MA, U.S.). The desired amounts of tRNAs were then mixed and precipitated again by ethanol. The pellets were air dried and stored at −80° C. until use.

### Example 3

### Cell-Free In Vitro Translation

The cell-free in vitro translation mix was prepared according to the previously reported method [Terasaka, N., Hayashi, G., Katoh, T., and Suga, H. (2014). An orthogonal ribosome-tRNA pair via engineering of the peptidyl transferase center. Nat. Chem. Biol. 10, 555-557]with the following modifications: recombinant IF1, IF2, IF3, EF-Ts, EF-Tu, EF-G, RF-2, RF-3, RRF, and MTF proteins were expressed in the E. coli strain BL21 with an N-terminal TEV-cleavable His-tag, purified by Ni-NTA Superflow resin (Senhui Microsphere Tech., Suzhou, China), cleaved by the TEV protease (Sigma-Aldrich, MO, U.S.), further purified by ion-exchange chromatography, and exchanged into a buffer containing 50 mM HEPES at pH 7.6, 100 mM potassium glutamate, 10 mM magnesium acetate, 7 mM P-mercaptoethanol, and 30% glycerol. Buffer components and small molecule ingredients were prepared as described in the literature [Goto, Y., Katoh, T., and Suga, H. (2011). Flexizymes for genetic code reprogramming. Nat. Protoc. 6, 779-790]. The aaRS-free E. coli ribosome was purchased from New England Biolabs (MA, U.S.).

### Example 4

### AaRS-Free Translation of Protein Enzymes

The 20-codon DNA templates for chicken lysozyme, Gaussia luciferase, and E. coli TrpRS were synthesized and assembled by Genewiz (Jiangsu, China) and cloned into the pUC-57 vector. Table 5 presents DNA template sequences for aaRS-free translation of chicken lysozyme, Gaussia luciferase, and E. coli TrpRS.

TABLE 5

| DNA templates | Sequence | SEQ ID No. |
|---|---|---|
| Chicken lysozyme | 5′- | 83 |
| | ATGAAGGTCTTCGGCCGGTGCGAGCTCGCCGCCGCCAT | |
| | GAAGCGGCACGGCCTCGACAACTACCGGGGCTACTCGC | |
| | TCGGCAACTGGGTCTGCGCCGCCAAGTTCGAGTCGAAC | |
| | TTCAACACGCAGGCCACGAACCGGAACACGGACGGCTC | |
| | GACGGACTACGGCATCCTCCAGATCAACTCGCGGTGGT | |
| | GGTGCAACGACGGCCGGACGCCGGGCTCGCGGAACCTC | |
| | TGCAACATCCCGTGCTCGGCCCTCCTCTCGTCGGACATC | |
| | ACGGCCTCGGTCAACTGCGCCAAGAAGATCGTCTCGGA | |
| | CGGCAACGGCATGAACGCCTGGGTCGCCTGGCGGAACC | |
| | GGTGCAAGGGCACGGACGTCCAGGCCTGGATCCGGGGC | |
| | TGCCGGCTCTAA-3′ | |
| Gaussia luciferase | 5′- | 84 |
| | ATGAAGCCGACGGAGAACAACGAGGACTTCAACATCGT | |
| | CGCCGTCGCCTCGAACTTCGCCACGACGGACCTCGACG | |
| | CCGACCGGGGCAAGCTCCCGGGCAAGAAGCTCCCGCTC | |
| | GAGGTCCTCAAGGAGATGGAGGCCAACGCCCGGAAGG | |
| | CCGGCTGCACGCGGGGCTGCCTCATCTGCCTCTCGCACA | |
| | TCAAGTGCACGCCGAAGATGAAGAAGTTCATCCCGGGC | |
| | CGGTGCCACACGTACGAGGGCGACAAGGAGTCGGCCC | |
| | AGGGCGGCATCGGCGAGGCCATCGTCGACATCCCGGAG | |
| | ATCCCGGGCTTCAAGGACCTCGAGCCGATGGAGCAGTT | |
| | CATCGCCCAGGTCGACCTCTGCGTCGACTGCACGACGG | |
| | GCTGCCTCAAGGGCCTCGCCAACGTCCAGTGCTCGGAC | |
| | CTCCTCAAGAAGTGGCTCCCGCAGCGGTGCGCCACGTT | |
| | CGCCTCGAAGATCCAGGGCCAGGTCGACAAGATCAAGG | |
| | GCGCCGGCGGCGACTAA-3′ | |
| E. coli TrpRS | 5′- | 85 |
| | ATGACGAAGCCGATCGTCTTCTCGGGCGCCCAGCCGTC | |
| | GGGCGAGCTCACGATCGGCAACTACATGGGCGCCCTCC | |
| | GGCAGTGGGTCAACATGCAGGACGACTACCACTGCATC | |
| | TACTGCATCGTCGACCAGCACGCCATCACGGTCCGGCA | |
| | GGACGCCCAGAAGCTCCGGAAGGCCACGCTCGACACG | |
| | CTCGCCCTCTACCTCGCCTGCGGCATCGACCCGGAGAA | |
| | GTCGACGATCTTCGTCCAGTCGCACGTCCCGGAGCACG | |
| | CCCAGCTCGGCTGGGCCCTCAACTGCTACACGTACTTCG | |
| | GCGAGCTCTCGCGGATGACGCAGTTCAAGGACAAGTCG | |
| | GCCCGGTACGCCGAGAACATCAACGCCGGCCTCTTCGA | |
| | CTACCCGGTCCTCATGGCCGCCGACATCCTCCTCTACCA | |
| | GACGAACCTCGTCCCGGTCGGCGAGGACCAGAAGCAG | |
| | CACCTCGAGCTCTCGCGGGACATCGCCCAGCGGTTCAA | |
| | CGCCCTCTACGGCGAGATCTTCAAGGTCCCGGAGCCGTT | |
| | CATCCCGAAGTCGGGCGCCCGGGTCATGTCGCTCCTCGA | |
| | GCCGACGAAGAAGATGTCGAAGTCGGACGACAACCGG | |
| | AACAACGTCATCGGCCTCCTCGAGGACCCGAAGTCGGT | |
| | CGTCAAGAAGATCAAGCGGGCCGTCACGGACTCGGACG | |
| | AGCCGCCGGTCGTCCGGTACGACGTCCAGAACAAGGCC | |
| | GGCGTCTCGAACCTCCTCGACATCCTCTCGGCCGTCACG | |
| | GGCCAGTCGATCCCGGAGCTCGAGAAGCAGTTCGAGGG | |
| | CAAGATGTACGGCCACCTCAAGGGCGAGGTCGCCGACG | |
| | CCGTCTCGGGCATGCTCACGGAGCTCCAGGAGCGGTAC | |
| | CACCGGTTCCGGAACGACGAGGCCTTCCTCCAGCAGGT | |
| | CATGAAGGACGGCGCCGAGAAGGCCTCGGTCCACGCCT | |
| | CGCGGACGCTCAAGGCCGTCTACGAGGCCATCGGCTTC | |
| | GTCGCCAAGCCGTAA-3′ | |

The DNA plasmids were double-digested and purified by 1% agarose gel prior to use. Upon retrieval from −80° C., the dried flexizyme-charged tRNA pellets were washed twice with 70% ethanol, and dissolved in 10-20 l of 1 mM NaOAc at pH 5.2. The dissolved tRNA mix was then added to the aaRS-free translation mix that had been pre-incubated at 37° C. for 5 min, with the final DNA template concentration at ˜10 ng/l. For the translation of lysozyme and luciferase, ˜1 μM of flexizyme-charged tRNAs were used for each translated codon; for the translation of TrpRS, ˜1 μM of flexizyme-charged FAM-labeled Fph-tRNA^{fMet}, ˜0.4 μM of flexizyme-charged tRNAs for each Cys and Pro codon, and ˜0.2 μM of flexizyme-charged tRNAs for each remaining codon were used (overall tRNA concentrations are provided in Table 3 hereinabove). The control experiments without DNA template were performed using identical flexizyme-charged tRNA concentrations, whereas the control experiments with uncharged tRNAs used 30 μM (each) for tRNA^{Asn}, tRNA^{Glu}, tRNA^{LYs}, t RNA^{Ile}, and 5 μM (each) for the other tRNAs, as well as 100 μM (each) for the free amino acids. Translation reactions were incubated at 37° C. for 2 hr for lysozyme and luciferase and 4 hr for TrpRS. For the analysis by 15% SDS-PAGE, a 10 l aliquot was sampled from the translation reaction, mixed with 2 μl of 6 × protein loading dye and heated at 98° C. for 3 min for loading. The Alexa Fluor 488-labeled Benchmark fluorescent protein standard was purchased from Thermo Fisher Scientific (MA, U.S.). The gels were scanned by Typhoon FLA 9500 (GE Healthcare, U.K.) operated under Cy2 mode.

### Example 5

### Biochemical Characterization of aaRS-Free Translated Protein Enzymes

AaRS-free translation of chicken lysozyme, Gaussia luciferase, and E. coli TrpRS were performed with Met-tRNA^{fMet}. The translation mix for chicken lysozyme was diluted with an equal volume of a 2×folding buffer containing 0.1 μM sodium phosphate and 0.1 μM NaCl at pH 7.5, incubated for 24 hr at room temperature, and assayed by the EnzChek Lysozyme Assay Kit (Thermo Fisher Scientific, MA, U.S.). The translation mix for Gaussia luciferase was diluted with an equal volume of a 2 × folding buffer containing 6 mM reduced and 4 mM oxidized glutathione at pH 7.3, shown previously to facilitate disulfide bond formation in recombinant Gaussia luciferase [Yu, T., Laird, J.R., Prescher, J. A., and Thorpe, C. (2018). Gaussia princeps luciferase: a bioluminescent substrate for oxidative protein folding. Protein Science 27, 1509-1517], incubated for 16 hr at room temperature, and assayed by the Pierce Gaussia Luciferase Glow Assay Kit (Thermo Fisher Scientific, MA, U.S.) according to manufacture's instructions. For the translation of E. coli TrpRS, Cy5-tRNA^{Trp} was prepared by enzymatic ligation of two synthetic oligos, and purified by 10% denaturing PAGE as described in the literature [Suddala, K. C., Cabello-Villegas, J., Michnicka, M., Marshall, C., Nikonowicz, E. P., and Walter, N. G. (2018). Hierarchical mechanism of amino acid sensing by the T-box riboswitch. Nat. Commun. 9, 1896]. Table 6 presents RNA oligo sequences for enzymatic ligation of internally Cy5 labeled tRNA^{Tr}.

TABLE 6

| Oligo | Modifications | Sequence | SEQ ID No. |
|---|---|---|---|
| Oligo 1 | Unmodified | 5′- | 86 |
| | | aggggcguaguucaauugguagagcaccgg | |
| | | ucucc-3′ | |
| Oligo 2 | 5′-phosphorylated | 5′-p-aaaaccgggu-(dT-Cy5)- | 87 |
| | and internally | uugggaguucgagucucuccgccccugcc | |
| | Cy5-labeled | a-3′ | |

### Substitute Specification

A mixture of 2 μM Cy5-tRNA^{Trp}, 250 μM tryptophan, and 1 mM ATP was added to the reaction mix after completion of translation, incubated for 1 hr at 37° C., quenched by 0.3 μM NaOAc, and phenol-chloroform extracted. The charged samples were analyzed by 8% acid PAGE (Supplemental Information) and scanned by Typhoon FLA 9500 operated under Cy5 mode. A sample of 2 μM of uncharged Cy5-tRNA^{Trp} and 2 μM of Cy5-tRNA^{Trp} charged by 100 nM of recombinant E. coli TrpRS were used as standards. All control experiments lacking DNA template, or with uncharged tRNAs and free amino acids, were assayed under identical conditions.

### Example 6

### Maximizing the Yield of aaRS-Free Translation

The aaRS-free translation system was studied to address the apparent low yield issue reported in the literature [Terasaka, N., Hayashi, G., Katoh, T., and Suga, H. (2014). An orthogonal ribosome-tRNA pair via engineering of the peptidyl transferase center. Nat. Chem. Biol. 10, 555-557]. The rationale was that the yield of aaRS-free translation might be improved by increasing the concentrations of flexizyme-charged tRNAs to compensate for the lack of tRNA recycling. Earlier studies showed that adding excessive tRNAs in Escherichia coli (E. coli) translation systems with aaRS inhibited the translation [Rojiani, M. V., Jakubowski, H., and Goldman, E. (1990). Relationship between protein synthesis and concentrations of charged and uncharged tRNA^{Trp} in Escherichia coli. Proc. Natl Acad. Sci. USA 87, 1511; Anderson, W. F. (1969). The effect of tRNA concentration on the rate of protein synthesis. Proc. Natl Acad. Sci. USA 62, 566], which was attributed to uncharged tRNAs competing out the charged tRNAs by occupying the ribosomal A-site, or cation imbalance from the addition of large amounts of tRNAs. However, all of these experiments were performed in the presence of aaRS, and thus the exact charging yields were not determined, making it difficult to differentiate between the influence of inefficient charging and altered cation (e.g., Mga+) concentrations.

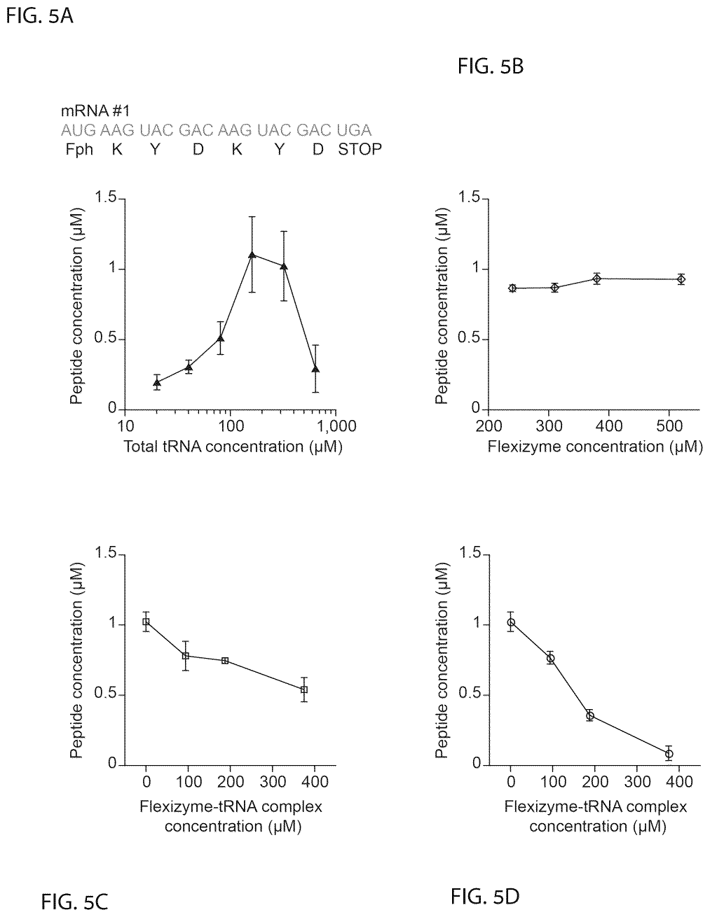

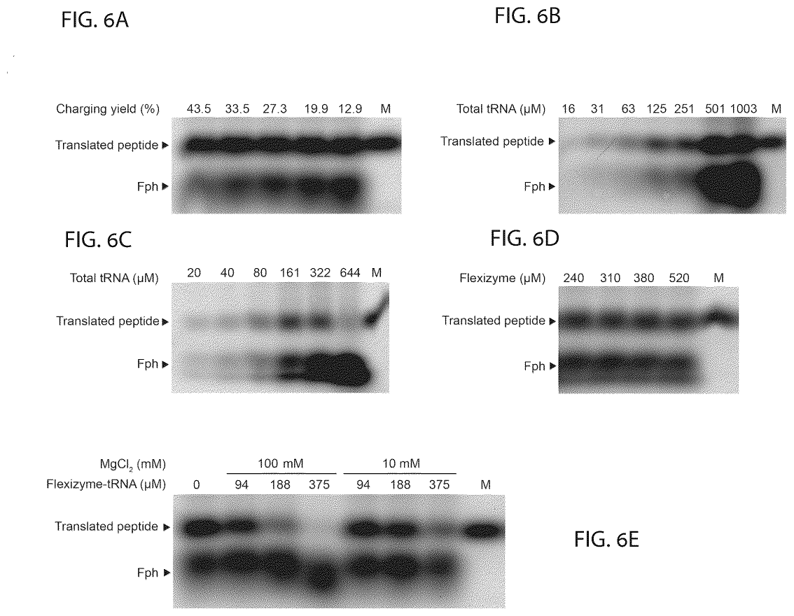

To evaluate the effect of tRNA concentrations and charging yields on aaRS-free translation, the aaRS-free translation of a short peptide using a fluorescein (FAM)-labeled phenylalanine (Fph-tRNA^{fMet}) was performed to facilitate the quantification of translation yields (see, FIGS. 4A-B, FIG. 5A-D and FIG. 6A-E).

FIGS. 4A-G present the results of an aaRS-free translation of multiple short peptides, according to some embodiments of the present invention, showing MALDI-TOF-MS analysis of translated short peptides from mRNA #1 (FIG. 4A), aaRS-free translation yield of short peptides, analyzed by Tricine-SDS-PAGE, showing uncharged tRNA concentrations ranged from 160-540 μM while the flexizyme-charged tRNA concentration remained at 70 uM, resulting in charging yields ranging from 44-13% (upper part of FIG. 4B), and total tRNA concentrations ranged from 16-1003 μM while the charging yield remained at 56% (lower part of FIG. 4B)(error bar represents standard deviations from three independent experiments), MALDI-TOF-MS analysis of translated short peptides from mRNA #2 (FIG. 4C), mRNA #3 (FIG. 4D), mRNA #4 (FIG. 4E), mRNA #5 (FIG. 4F), and mRNA #6 (FIG. 4G), with control of translation with 55-135 μM of total uncharged tRNAs and 100 μM of each amino acid (for mRNA #1, 5 μM of flexizyme-charged FAM-labeled Fph-tRNA^{fMet} was added to both the control and aaRS-free translation experiments), and aaRS-free translation of aaRS-free translation with 170-414 μM of total flexizyme-charged tRNAs (see, Table 3). a.u., arbitrary units; C, O: calculated and observed m/z values, (FIGS. 4C-G, respectively).

The mRNA template was decoded by tRNA^{fMet}, tRNA^{LYs}, tRNA^{Tyr}, and tRNA^{Asp}, among which tRNA^{fMet} was charged with Fph-tRNA^{fMet} by the 45-nt enhanced flexizyme, and the others were charged with their cognate amino acids by the 46-nt dinitro-flexizyme μMurakami, H., Ohta, A., Ashigai, H., and Suga, H. (2006). A highly flexible tRNA acylation method for non-natural polypeptide synthesis. Nat. Methods 3, 357]. Unmodified tRNAs transcribed in vitro by the T7 RNA polymerase were used as they have been shown to operate in ribosomal peptide synthesis assays. The individual charging yield for each tRNA was determined by polyacrylamide gel electrophoresis under acidic conditions (acid PAGE), which was used to deduce the weighted average (overall) charging yield of the translation system (see, Table 3). Titration of tRNA was first performed by adding charged total tRNAs with an overall charging yield of about 44% (mixed Fph-tRNA^{fMet}: Lys-tRNA^{Lys}: Tyr-tRNA^{Tyr}: Asp-tRNA^{Asp} at 1:2:2:2 molar ratio), and total tRNA concentrations from 20-644 μM in the final translation system, and discovered that the translation yield reached the highest level when the total tRNA concentration was at about 160 μM, and without plateauing, the translation yield decreased upon further increases of tRNA concentrations (see, FIG. 5A).

FIGS. 5A-D present results of aaRS-free translation of mRNA #1 under various conditions, showing total tRNA concentrations ranged from 20-644 μM, with charging yield remained at 44% (FIG. 5A), total flexizyme concentrations ranged from 240-525 μM, with total tRNA concentration remained at 160 μM (FIG. 5B), flexizyme and uncharged tRNAs from 0-380 μM were mixed in (FIG. 5C) 10 mM MgCl₂ and (FIG. 5D) 100 mM MgCl₂, desalted by ethanol precipitation, and added to the aaRS-free translation mix, wherein the concentration of flexizyme-charged tRNA was remained at 70 μM (error bar, standard deviations from three independent experiments).

FIGS. 6A-D present tricine-SDS-PAGE gel analysis for calculating the aaRS-free translation yields, showing gel images corresponding to FIG. 4A, FIG. 4B, FIG. 5A, FIG. 5B, FIG. 5C, and FIG. 5D (FIGS. 6A-E respectively), for calculating the aaRS-free translation yields, wherein “M” is a synthetic peptide standard (Fph-K-Y-D-K-Y-D).

The observed inhibition was not attributed to the flexizyme buildup in the translation system, because in a control experiment, the addition of purified dinitro-flexizyme to a fixed amount of total tRNAs did not inhibit the translation (see, FIG. 5B).

Next, 90-470 μM of uncharged tRNAs were added to the aaRS-free translation system in the presence of 70 μM charged tRNAs, with the overall charging yield decreased from 44 to 13%, but the overall translation yield remained largely unaffected (see, FIG. 4B).

It was reasoned that another factor that could be responsible for the observed translation inhibition was the increased cation concentration due to Mg²⁺carryover from the flexizyme-charged tRNAs. To test this theory, exogenous MgCl₂ was added to the flexizyme-charged tRNAs before addition to the aaRS-free translation system, and it was discovered that the translation was indeed inhibited by increased MgCl₂ carryover (see, FIG. 5C and FIG. 5D).

Based on the aforementioned observation, high-performance liquid chromatography (HPLC) equipped with a C18 column was used to purify the flexizyme-charged tRNAs to reduce the Mg²⁺concentrations (except Fph-tRNA^{fMet} which was treated by ultrafiltration instead to minimize fluorescence quenching). This process also removed most of the flexizyme and modestly improved the overall charging yield from 44 to 56% (see, FIG. 2).

The purified, flexizyme-charged tRNAs was thereafter added to the aaRS-free translation system and it was observed that the translation yield was significantly improved by 5-fold as a result of concentrating the flexizyme-charged tRNAs alone. An additional 2-fold improvement was observed upon reducing the Mg²⁺contamination by HPLC, resulting in a about 10-fold overall improvement of translation yield (see, FIG. 4B and FIG. 5A), with the optimal total tRNA concentration shifted from 160 to 500 μM.

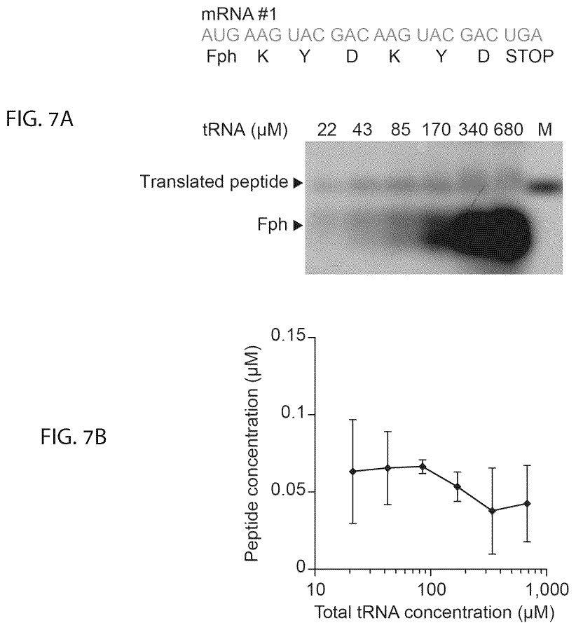

However, when tRNA concentrations further increased from 500 to 1000 μM, the overall translation yield reduced by about 50%, potentially resulting from the Mg²⁺associated with Fph-tRNA^{fMet}. Moreover, similar titration assays with high concentrations of uncharged tRNAs in the presence of aaRS did not lead to improvement in translation yield (see, FIG. 7A-B), suggesting that the improvement of aaRS-free translation yield likely resulted from the increased concentrations of flexizyme-charged tRNAs per se.

FIGS. 7A-B presents the results of an in vitro translation experiment in the presence of LysRS, TyrRS and AspRS, showing tricine-SDS-PAGE analysis of translation products with uncharged, unmodified total tRNA concentrations ranging from 22-680 μM in the presence of LysRS, TyrRS and AspRS, and Fph-tRNAfMet pre-charged by enhanced flexizyme (FIG. 7A, and the calculated translation yield (FIG. 7B)(error bar, standard deviations from three independent experiments).

### Example 7

### AaRS-Free Translation of Multiple Short Peptides

Having discovered that increasing tRNA concentrations improved the yield of aaRS-free translation, the present inventors have sought to test the aaRS-free translation on multiple short peptides and determine the translation fidelity under high flexizyme-charged tRNA concentrations. A minimal set of 21 E. coli tRNAs was obtained through in vitro transcription by the T7 RNA polymerase, including 1 tRNA (tRNA^{fMet}) for translation initiation and 20 others for translation elongation. Table 7 presents the relevant tRNA sequences.

TABLE 7

| tRNA | Anticodon | Sequence | SEQ ID No. |
|---|---|---|---|
| Ala | GGC | 5′- | 88 |
| | | ggggcuauagcucagcugggagagcgcuugcauggc | |
| | | augcaagaggucagcgguucgaucccgcuuagcucc | |
| | | acca-3′ | |
| Arg | CCG | 5′- | 89 |
| | | gcgcccguagcucagcuggauagagcgcugcccucc | |
| | | ggaggcagaggucucagguucgaauccugucgggcg | |
| | | cgcca-3′ | |
| Asn | GUU | 5′- | 90 |
| | | uccucuguaguucagucgguagaacggcggacuguu | |
| | | aauccguaugucacugguucgaguccagucagagga | |
| | | gcca-3′ | |
| Asp | GUC | 5′- | 91 |
| | | ggagcgguaguucagucgguuagaauaccugccugu | |
| | | cacgcagggggucgcggguucgagucccguccguuc | |
| | | cgcca-3′ | |
| Cys | GCA | 5′- | 92 |
| | | ggcgcguuaacaaagcgguuauguagcggauugcaa | |
| | | auccgucuaguccgguucgacuccggaacgcgccuc | |
| | | ca-3′ | |
| fMet | CAU | 5′- | 93 |
| | | gggggguggagcagccugguagcucgucgggcuca | |
| | | uaacccgaagaucgucgguucaaauccggcccccgca | |
| | | acca-3′ | |
| Gln | CUG | 5′- | 94 |
| | | ugggguaucgccaagcgguaaggcaccggauucuga | |
| | | uuccggcauuccgagguucgaauccucguaccccag | |
| | | cca-3′ | |
| Glu | CUC | 5′- | 95 |
| | | gtccccuucgucuagaggcccaggacaccgcccucuc | |
| | | acggcgguaacagggguucgaauccccuaggggacg | |
| | | cca-3′ | |
| Gly | GCC | 5′- | 96 |
| | | gcgggaauagcucaguugguagagcacgaccuugcc | |
| | | aaggucggggucgcgaguucgagucucguuucccgc | |
| | | ucca-3′ | |
| His | GUG | 5′- | 97 |
| | | guggcuauagcucaguugguagagcccuggauugu | |
| | | gauuccaguugucguggguucgaaucccauuagcca | |
| | | cccca-3′ | |
| Ile | GAU | 5′- | 98 |
| | | gggcuuguagcucaggugguuagagcgcaccccuga | |
| | | uaagggugaggucggugguucaaguccacucaggcc | |
| | | cacca-3′ | |
| Leu | GAG | 5′- | 99 |
| | | gccgaggugguggaauugguagacacgcuaccuuga | |
| | | ggugguagugcccaauagggcuuacggguucaaguc | |
| | | ccguccucgguacca-3′ | |
| Lys | CUU | 5′- | 100 |
| | | gggucguuagcucaguugguagagcaguugacucu | |
| | | uaaucaauuggucgcagguucgaauccugcacgacc | |
| | | cacca-3′ | |
| Met | CAU | 5′- | 101 |
| | | ggcuacguagcucaguugguuagagcacaucacuca | |
| | | uaaugauggggucacagguucgaaucccgucguagc | |
| | | cacca-3′ | |
| Phe | GAA | 5′- | 102 |
| | | gcccggauagcucagucgguagagcaggggauugaa | |
| | | aauccccguguccuugguucgauuccgaguccgggc | |
| | | acca-3′ | |
| Pro | CGG | 5′- | 103 |
| | | cggugauuggcgcagccugguagcgcacuucguucg | |
| | | ggacgaaggggucggagguucgaauccucuaucacc | |
| | | gacca-3′ | |
| Ser | CGA | 5′- | 104 |
| | | ggagagaugccggagcggcugaacggaccggucucg | |
| | | aaaaccggaguaggggcaacucuaccggggguucaa | |
| | | aucccccucucuccgcca-3′ | |
| Thr | CGU | 5′- | 105 |
| | | gccgauauagcucaguugguagagcagcgcauucgu | |
| | | aaugcgaaggucguagguucgacuccuauuaucggc | |
| | | acca-3′ | |
| Trp | CCA | 5′- | 106 |
| | | aggggcguaguucaauugguagagcaccggucucca | |
| | | aaaccggguguugggaguucgagucucuccgccccu | |
| | | gcca-3′ | |
| Tyr | GUA | 5′- | 107 |
| | | ggugggguucccgagcggccaaagggagcagacugu | |
| | | aaaucugccgucaucgacuucgaagguucgaauccu | |
| | | ucccccaccacca-3′ | |
| Val | GAC | 5′- | 108 |
| | | gcguccguagcucaguugguuagagcaccaccuuga | |
| | | cauggugggggucggugguucgaguccacucggac | |
| | | gcacca-3′ | |

Each tRNA was separately charged by the flexizyme with charging yields ranging from 20-60% (see, FIG. 8A).

FIGS. 8A-B present flexizyme-charging yields of 21 tRNAs with their cognate proteinogenic amino acids, showing the charging yield determined after ethanol precipitation (FIG. 8A), and the charging yield determined after HPLC purification of 14 flexizyme-charged tRNAs. N/A, purification of flexizyme-charged tRNAs not performed (FIG. 8B), wherein the reversible N-pentenoylation was performed for gly-tRNA^{G}lY to facilitate the purification as reported previously.

The flexizyme-charged tRNAs were mixed at a molar ratio according to the abundance of their cognate codons on the mRNA before being added to the aaRS-free translation system to a final concentration ranging from 170-520 μM (Table 3). The inventors designed and in vitro transcribed five distinct mRNA sequences that allowed Watson-Crick base pairing to the anticodon of flexizyme-charged tRNAs, and the aaRS-free translated short peptides were evaluated by matrix-assisted laser desorption ionization-time of flight mass spectrometry (MALDI-TOF MS) to test the translation fidelity (see, FIGS. 4C-G).

The MALDI-TOF MS results showed that all 21 flexizyme-charged tRNAs accurately decoded the mRNAs with up to -200-fold molar excess over the ribosome (e.g., 414 μM tRNAs v. s. 2 μM ribosome with mRNA #5). In the control experiments with uncharged tRNAs and free amino acids (see, FIGS. 4C-G), no peptide products were detected, thus minimizing contamination concerns of aaRS and charged tRNAs from ribosome preparation.

Notably, the inventors encoded a short message “MITRNACHARGINGSYSTEM” (SEQ ID No. 123) into mRNA #6 (see, FIG. 4G) and successfully translated the full-length information-carrying peptide.

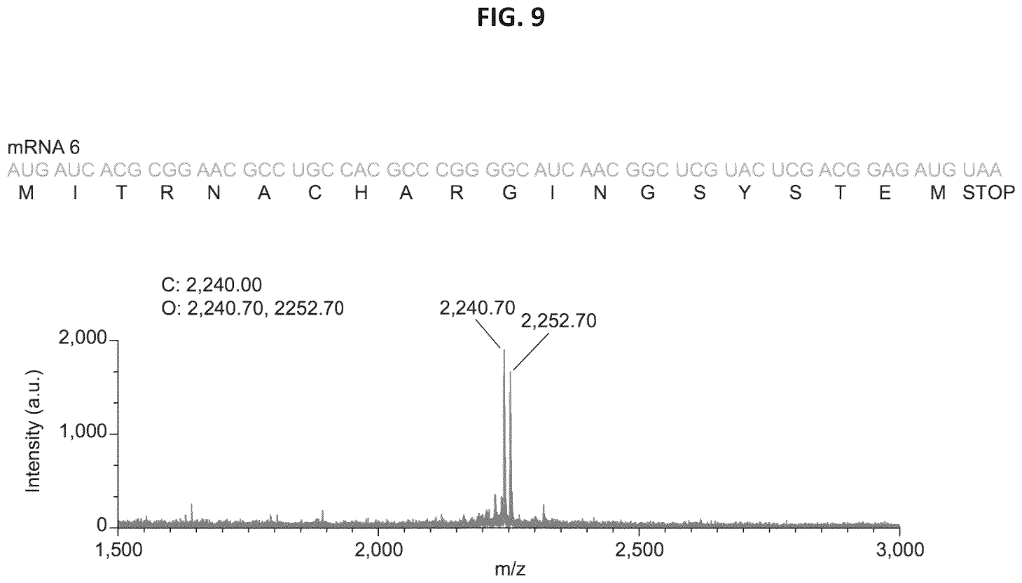

However, when the total tRNA concentration was increased to 520 μM, an additional+12 Da peak was detected (see, FIG. 9), potentially due to mRNA misdecoding resulting from the high tRNA concentration and use of unmodified tRNAs for translation.

FIG. 9 presents MALDI-TOF MS analysis of aaRS-free translated mRNA #6, showing that with a higher total tRNA concentration (520 μM) in the aaRS-free translation system, a mistranslated product was observed with a M.W. of 2,252.7 Da, whereas the correctly translated product had a M.W. of 2,240.7 Da. a.u., arbitrary units; C, O: calculated and observed m/z values, respectively.

### Example 8

### AaRS-Free Translation of Protein Enzymes

The successful translation of short peptides led to the aaRS-free translation of protein enzymes exclusively with in vitro transcribed, unmodified tRNAs charged by the flexizyme system. Two small enzymes, the 130-aa chicken lysozyme and the 169-aa Gaussia lucifierase, were chosen as models. Neither of the enzymes are native to E. coli and thus minimizes contamination concerns from ribosome preparation.

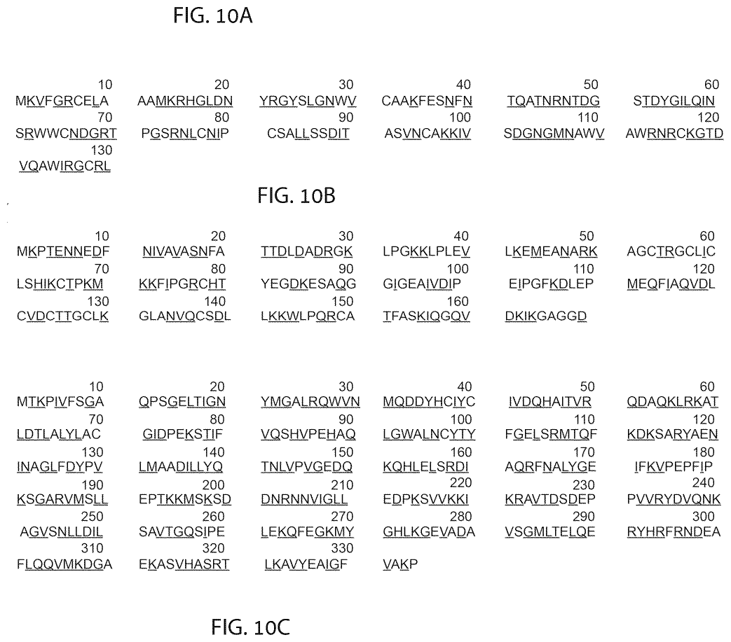

FIGS. 10A-C present the amino acid sequences of aaRS-free translated protein enzymes: chicken lysozyme (FIG. 10A), Gaussia luciferase (FIG. 10B), and E. coli TrpRS (FIG. 10C), whereas positions translated by the flexizyme-charged tRNAs were purified either by ethanol precipitation or by HPLC (underlined).

A subset (underlined amino acids in FIGS. 10A-B) of the 21 flexizyme-charged tRNAs were purified by HPLC to reduce Mg²⁺carryover, and the individual charging yields after HPLC purification were determined by acid PAGE (see, FIG. 8B), resulting in an overall charging yield of about 40%. The total tRNA concentration of about 330 μM for chicken lysozyme and about 430 M for Gaussia luciferase (Table 3) was approximately 10- to 20-fold higher than those used in other in vitro translation systems [Terasaka, N., Hayashi, G., Katoh, T., and Suga, H. (2014). An orthogonal ribosome-tRNA pair via engineering of the peptidyl transferase center. Nat. Chem. Biol. 10, 555-557; Iwane, Y., Hitomi, A., Murakami, H., Katoh, T., Goto, Y., and Suga, H. (2016). Expanding the amino acid repertoire of ribosomal polypeptide synthesis via the artificial division of codon boxes. Nat. Chem. 8, 317-325; and Cui, Z., Stein, V., Tnimov, Z., Mureev, S., and Alexandrov, K. (2015). Semisynthetic tRNA complement mediates in vitro protein synthesis. J. Am. Chem. Soc. 137, 4404-4413].

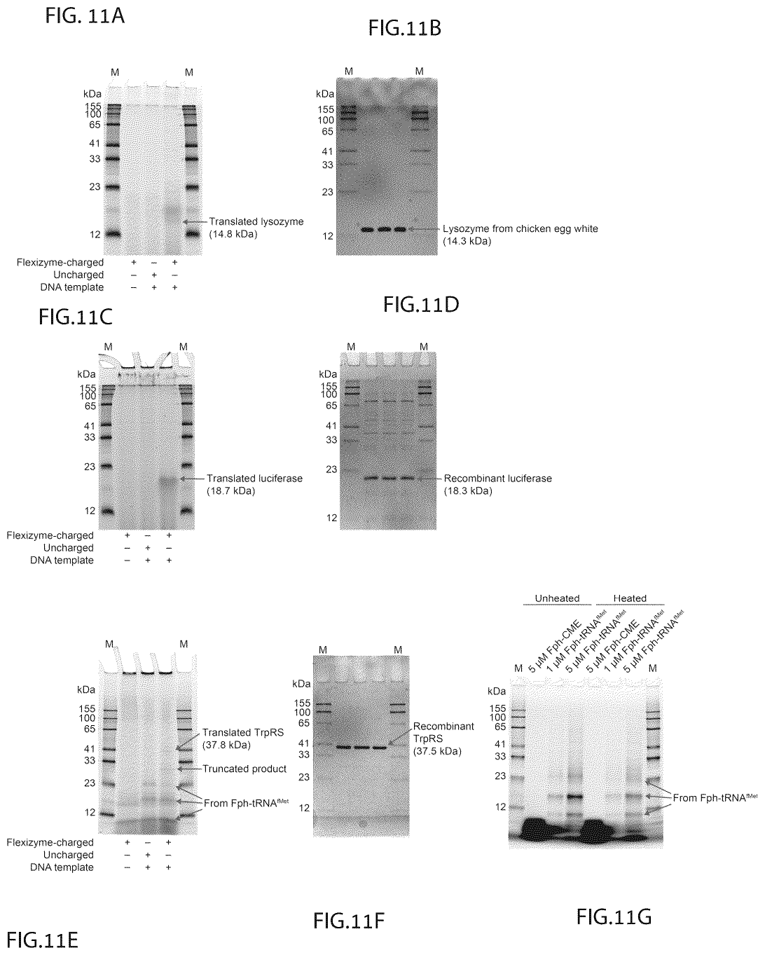

The aaRS-free translation of the full-length proteins was tested using the FAM-labeled Fph-tRNA^{fMet} reporter. Analyzed by sodium dodecyl sulfate-polyacrylamide gel electrophoresis (SDS-PAGE), the fluorescently labeled product bands were consistent with the molecular weight of the chicken lysozyme and Gaussia luciferase (14.8 kDa and 18.7 kDa, respectively), and the mobility of the product bands was also similar to that of commercial chicken lysozyme and recombinant Gaussia luciferase, respectively (see, FIGS. 11A-D).

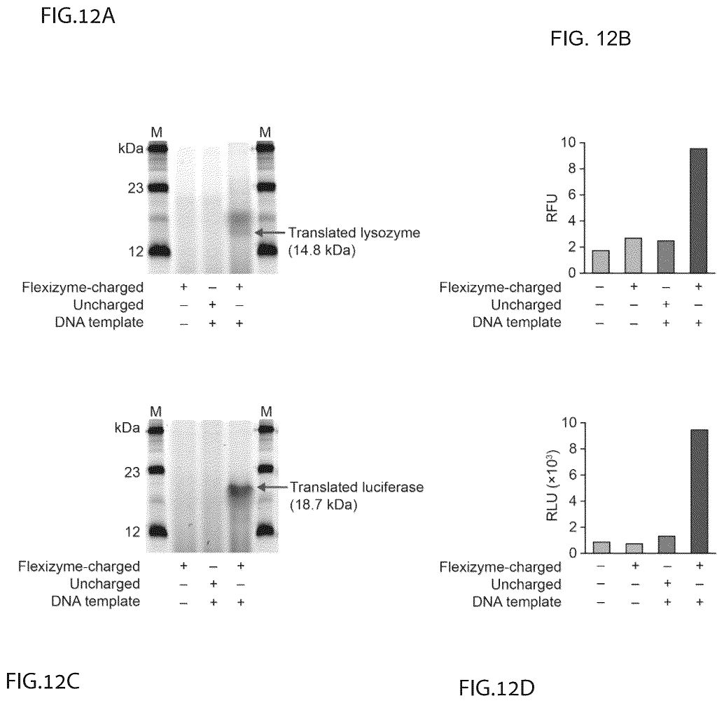

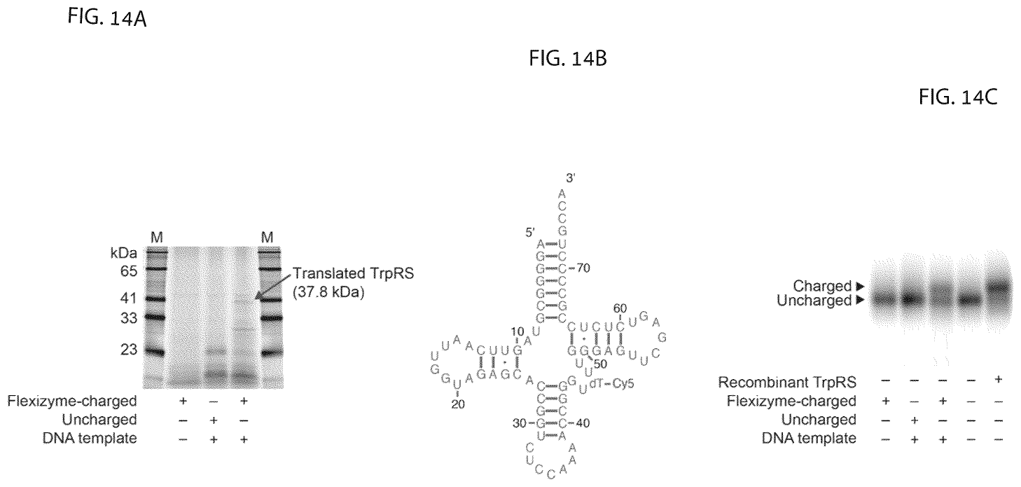

FIGS. 11A-G present SDS-PAGE analysis of aaRS-free translated protein enzymes, showing the entire gel image shown in FIG. 12A (FIG. 11A), a samples of 400 ng commercial chicken lysozyme purified from chicken egg white that were analyzed in 15% SDS-PAGE, and stained by Coomassie Brilliant Blue (FIG. 11B), the entire gel image shown in FIG. 12C (FIG. 11C), samples of 400 ng recombinant Gaussia luciferase, expressed and purified from E. coli strain BL21 that were analyzed 15% SDS-PAGE, and stained by Coomassie Brilliant Blue (FIG. 11D), the entire gel image shown in FIG. 14A (FIG. 11E), samples of 300 ng recombinant E. coli TrpRS, expressed and purified from E. coli strain BL21 that were analyzed 15% SDS-PAGE, and stained by Coomassie Brilliant Blue (FIG. 11F), and samples of 5 μM Fph-CME, 1 μM Fph-tRNA^{fMet}, and 5 μM of Fph-tRNA^{fMet} that were analyzed by 15% SDS-PAGE with or without being heated to 98° C. for 3 min, and scanned by Typhoon FLA 9500 under Cy2 mode (FIG. 11G), wherein M is a benchmark fluorescent protein standard.

In comparison, the product bands were absent in the control experiments lacking DNA templates, or with uncharged tRNAs and free amino acids, respectively (see, FIG. 12A and FIG. 12C).

FIGS. 12A-D present results of experimental proof of concept of aaRS-free translation of protein enzymes, according to some embodiments of the present invention, showing aaRS-free translation of N-terminal FAM-labeled chicken lysozyme, analyzed by 15% SDS-PAGE, and scanned by Typhoon FLA 9500 under Cy2 mode (M represents a benchmark fluorescent protein standard) (FIG. 12A), enzymatic assay of crude aaRS-free translated chicken lysozyme, with fluorescently labeled bacterial (Micrococcus lysodeikticus) cell wall materials as substrates (FIG. 12B), aaRS-free translation of N-terminal FAM-labeled Gaussia luciferase, analyzed by 15% SDS-PAGE, and scanned by Typhoon FLA 9500 under Cy2 mode (FIG. 12C), and enzymatic assay of crude aaRS-free translated Gaussia luciferase, with coelenterazine as substrate (FIG. 12D)(RFU, relative fluorescence unit. RLU, relative luminescence unit).

These results suggested that aaRS-free translation was sufficiently processive to accomplish the synthesis of full-length proteins before the flexizyme-charged tRNAs were hydrolyzed. Attempts to characterize the aaRS-free translated proteins from the excised product bands using liquid chromatography-tandem mass spectrometry (LC-MS/MS) were unsuccessful due to ribosomal protein contamination; however, this was addressed by translating a larger protein with a molecular weight more different from those of the ribosomal proteins, as described herein. Next, the FAM-labeled Fph-tRNA^{fMet} was replaced with unlabeled Met-tRNA^{fMet} for translation initiation and performed enzymatic assays to test whether the translated proteins can fold correctly in vitro and possess their corresponding catalytic activities. The results showed that after incubation for up to 24 hr in the folding buffers, the aaRS-free translated enzymes carried out the catalysis of their corresponding substrates: the chicken lysozyme released FAM-labeled cell debris and the Gaussia luciferase emitted bioluminescence, respectively (see, FIG. 12B and FIG. 12D), whereas the control experiments lacking DNA templates, or with uncharged tRNAs and free amino acids, did not generate detectable signals, thus minimizing contamination concerns of auto-fluorescence or contaminating luminescence from the aaRS-free translation system.

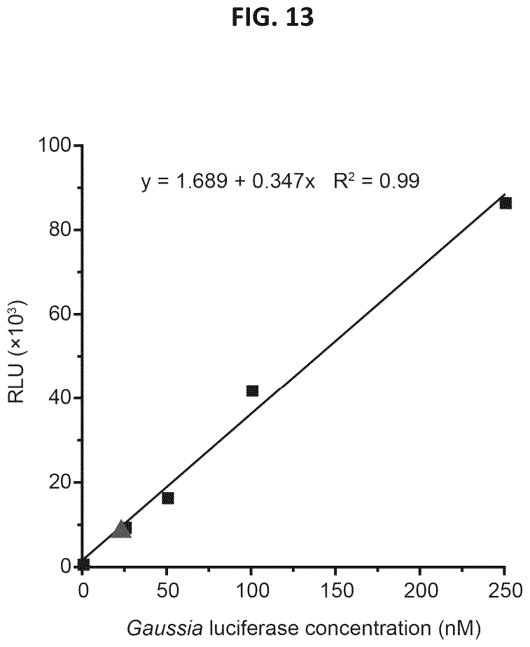

Comparing the emitted bioluminescence of the aaRS-free translated Gaussia luciferase with known standards of recombinant luciferase suggested a translation yield of about 25 nM (see, FIG. 13), which was about 80-fold lower than the maximal yield of the aaRS-free translation of a 7-aa peptide (see, FIG. 4B), likely as a result of the lower availability of flexizyme-charged tRNAs for each translated codon, as well as the limited folding efficiency of the Gaussia luciferase with multiple disulfide bonds.

FIG. 13 presents yield estimate values of aaRS-free translated Gaussia luciferase, wherein the standard curve plotted using 0, 25 nM, 50 nM, 100 nM, and 250 nM recombinant Gaussia luciferase (denoted by squares), and the yield of the translated Gaussia luciferase was estimated to be −25 nM (denoted by a triangle).

### Example 9

### AaRS-Free Translation of aaRS

The inventors sought to explore the possibility for the aaRS-free translation system to produce functional aaRS itself, an important step in establishing a self-reproducing translation apparatus. To that end, the 334-aa E. coli TrpRS was used as a model. A large portion (14 out of 21 in total) of the in vitro transcribed flexizyme-charged tRNAs were purified by HPLC to reduce Mg²⁺carryover (underlined amino acids in FIG. 10A), resulting in an overall charging yield of about 42% and total tRNA concentration of about 170 μM (see, Table 3).

The inventors used the FAM-labeled Fph-tRNA^{f}me reporter to test the translation of the full-length protein, and a product band indicative of the 334-aa E. coli TrpRS (37.8 kDa) was observed by SDS-PAGE (the mobility of the fluorescently labeled protein band was similar to that of recombinant TrpRS) (see, FIG. 11E and FIG. 11F), whereas this band was absent in the control experiments lacking DNA templates, or with uncharged tRNAs and free amino acids (see, FIG. 14A).

FIGS. 14A-C presents aaRS-free translation of TrpRS, showing aaRS-free translation of N-terminal FAM-labeled E. coli TrpRS, analyzed by 15% SDS-PAGE, and scanned by Typhoon FLA 9500 under Cy2 mode (M represents a benchmark fluorescent protein standard (FIG. 14A), sequence and secondary structure of internally Cy5-labeled tRNA^{Trp} (FIG. 14B), and enzymatic assay of crude aaRS-free translated TrpRS, with Cy5-tRNA^{Trp} as substrate, analyzed by 8% acid PAGE, and scanned by Typhoon FLA 9500 under Cy5 mode (FIG. 14C).

Also observed were several faster migrating bands, which may correspond to the truncated TrpRS translation products and unused Fph-tRNAMet (see, FIG. 11G).

To further confirm the aaRS-free translation of TrpRS, the protein content from the excised product band was analyzed using LC-MS/MS, and identified 4 non-overlapping peptide segments from E. coli TrpRS, resulting in a sequence coverage of about 16%. In comparison, no peptides corresponding to E. coli TrpRS were detected in the control experiment with uncharged tRNAs and free amino acids, suggesting that the detected TrpRS was not from endogenous aaRS contamination (Table 8).

Table 8 presents the detected peptide sequence of aaRS-free translated E. coli TrpRS by LC-MS/MS (aaRS-free translation with DNA template for E. coli TrpRS)

TABLE 8

| | SEQ | Position | | | MH+ | AM | RT |
|---|---|---|---|---|---|---|---|
| Segment sequence | ID No. | in protein | XCorr | Charge | [Da] | [ppm] | [min] |
| KATLDTLALYLAcGIDPEK | 109 | 58-76 | 3.60 | 3 | 2092.11179 | 5.84 | 61.22 |
| AVYEAIGFVAKP | 110 | 323-334 | 3.44 | 2 | 1264.70085 | 5.69 | 44.37 |
| AVTDSDEPPVVR | 111 | 223-234 | 3.15 | 2 | 1284.65019 | 5.53 | 24.40 |
| FNALYGEIFK | 112 | 164-173 | 2.72 | 2 | 1201.63152 | 5.23 | 49.75 |

To further test the tRNA-aminoacylating activity of the aaRS-free translated TrpRS, an internally Cy5-labeled tRNA substrate (Cy5-tRNA^{Trp}, see, FIG. 14B) was designed and synthesized.

Installation of a Cy5 label would allow in situ detection of the charged Cy5-tRNA^{Trp} without interference from other charged tRNA species. Using Met-tRNA^{fMet} for translation initiation, and after the TrpRS was translated, Cy5-tRNA^{Trp} was added along with tryptophan and adenosine triphosphate (ATP) to the aaRS-free translation system. The aaRS-free translated TrpRS successfully charged tryptophan onto Cy5-tRNA^{Trp}, whereas in the control experiments lacking DNA templates, or with uncharged tRNAs and free amino acids, no Cy5-tRNA^{Trp} charging was observed (see, FIG. 14C), suggesting that the observed Cy5-tRNA^{Trp} charging activity was unlikely due to endogenous aaRS contamination from ribosome preparation or residual flexizyme activities.

### Example 10

### AaRS-Free Charging of Mirror-Image tRNAs

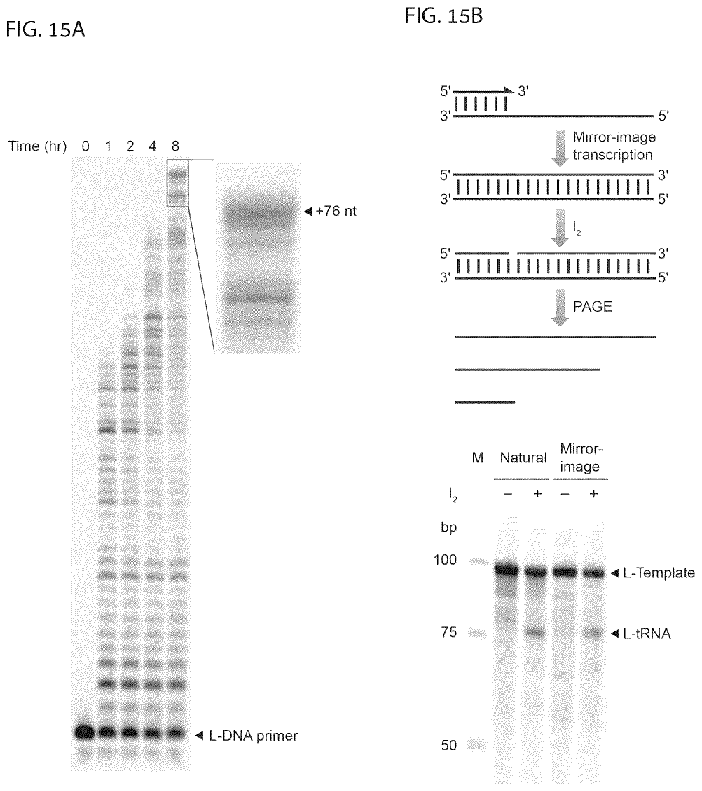

As a proof of concept experiment to test the charging of mirror-image L-tRNAs with mirror-image D-amino acids by a synthetic mirror-image L-flexizyme (see, FIG. 3A), the present inventors applied a previously established mirror-image gene transcription system based on the mirror-image version of a designed mutant of the Sulfolobus solfataricus P2 DNA polymerase IV (Dpo4) to transcribe the mirror-image tRNAs (see, FIG. 15A).

FIGS. 15A-B present results of the transcription of mirror-image tRNA^{Lys} by D-Dpo4-5m-Y12S, showing the extension of a 5′-FAM labeled L-universal primer on an L-ssDNA template, polymerized by the synthetic D-Dpo4-5m-Y12S polymerase, and the reaction aliquots that were terminated at different time points and analyzed by 12% denaturing PAGE gel in 7 μM urea (FIG. 15A), and showing mirror-image transcription and I2-mediated cleavage of the tRNA^{LYs} transcript, analyzed by 10% denaturing PAGE gel in 7 μM urea, and stained by SYBR-Green II by Thermo Fisher Scientific, MA, U.S. (FIG. 15B).

To avoid the high cost of synthesizing 21 different L-RNA primers, the inventors applied a universal primer for the transcription of mirror-image tRNAs (see, FIG. 15B).

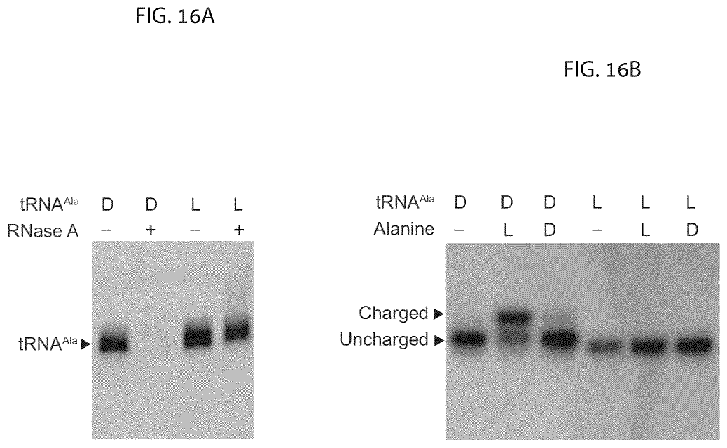

The universal primer was modified near the 3′ end by phosphorothioate so that the fully extended primers were efficiently cleaved by 12 via a previously reported mechanism, generating full-length mirror-image tRNAs (see, FIG. 15B), which were, as expected, resistant to natural RNase A digestion and unable to be charged by natural aaRS (see, FIGS. 16A-B).

FIGS. 16A-B present results of the biochemical characterization of enzymatically transcribed natural and mirror-image tRNAs, showing RNase A digestion of enzymatically transcribed D- and L-tRNA^{Ala} (FIG. 16A), and AaRS-catalyzed aminoacylation of enzymatically transcribed D- and L-tRNA^{Ala} (FIG. 16B).

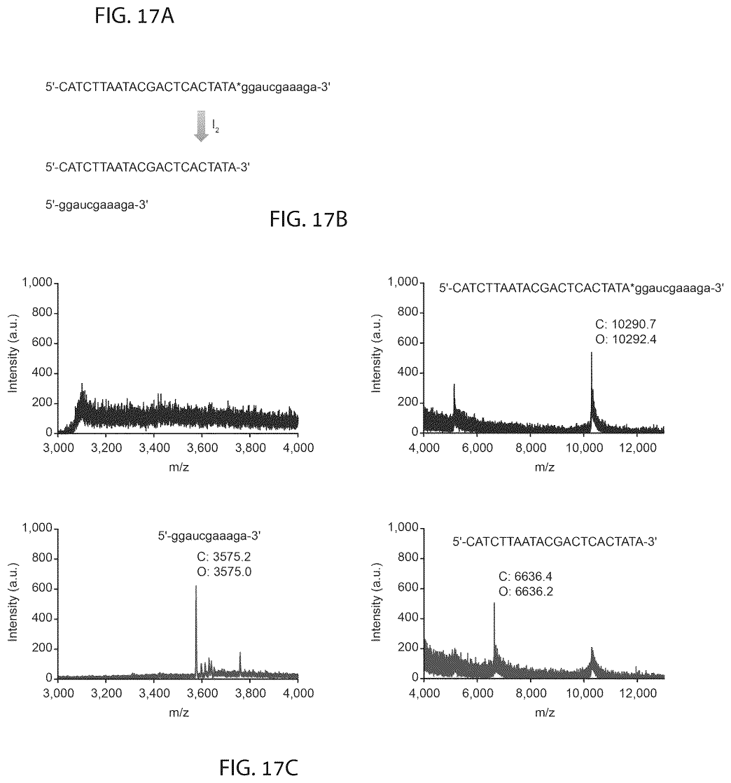

The I₂-mediated cleavage generates RNA with hydroxyl-terminated 5′-end, as verified by MALDI-TOF MS (see, FIGS. 17A-C).

FIGS. 17A-C present MALDI-TOF MS analysis of I₂-mediate cleavage, showing synthetic DNA-RNA chimeric oligo cleaved at the phosphorothioate modification site by 12 (FIG. 17A), MALDI-TOF MS spectrum of the uncleaved oligo under negative linear mode (FIG. 17B), MALDI-TOF MS spectrum of I₂-cleaved oligo under negative linear mode (m/z >4000) and negative reflectron mode (m/z<4000) (FIG. 17C), wherein the upper-case letters denote DNA nucleotides, lower-case letters denote RNA nucleotides, “*” denotes phosphorothioate modification. a.u., arbitrary units; C, O, calculated and observed m/z values, respectively. Next, a chemically synthesized 46-nt L-flexizyme (dinitro-flexizyme) was successfully used to charge 4 representative D-amino acids (lysine, alanine, glycine, and phenylalanine) that belong to different amino acid categories (polar (Lys), nonpolar (Ala), achiral (Gly), and aromatic (Phe), respectively) to their cognate mirror-image tRNAs, with similar efficiencies comparable to those of the natural system (see, FIGS. 3B-E).

### Example 11

### Translation of Complete or Partial Unnatural Peptides Using Cation-Depleted Flexizyme-Charged tRNAs

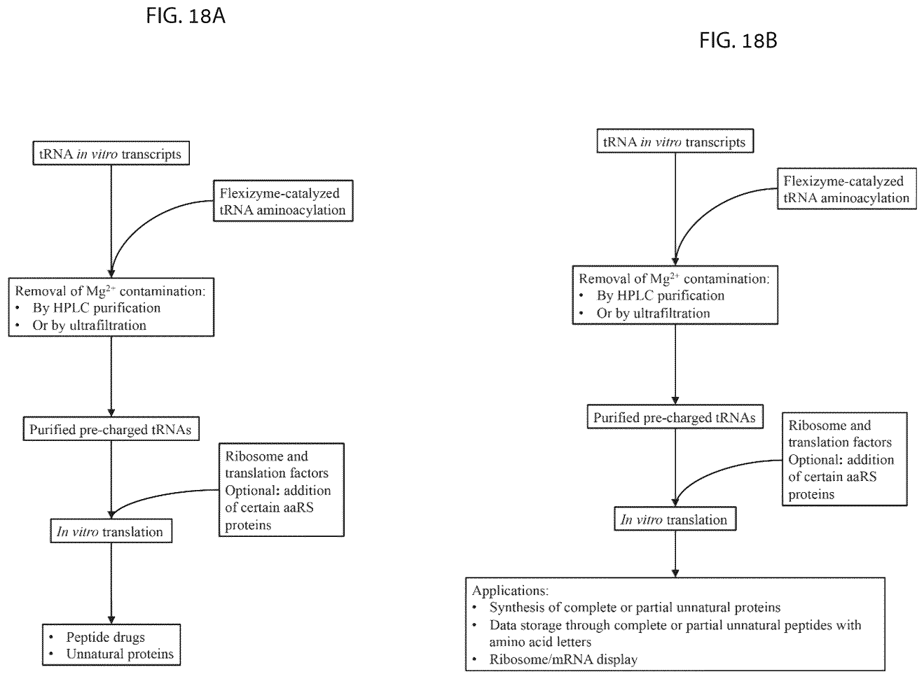

It was reasoned that the flexizyme system can be used to incorporate multiple unnatural amino acids for peptide translation, used in conjunction with or without other aaRS proteins. The provisions of the present invention allows to test whether unnatural peptides could be translated using the cation-depleted flexizyme-charged tRNAs or at least a preparation of flexizyme-charged tRNAs wherein the concentration of Mg⁺² is reduced essentially to minimal level possible, and whether purification by means such as HPLC and ultrafiltration, and concentrating the cation-depleted flexizyme-charged tRNAs could increase translation yield especially for the difficult-to-translate peptides such as complete or partial unnatural peptides. In the translation system, aaRS proteins may be added to enhance the charging of certain tRNAs not being charged by flexizymes (see, FIGS. 18A-B).

FIGS. 18A-B present flow charts translation of complete or partial unnatural peptides using cation-depleted flexizyme-charged tRNAs, showing translation of peptide drugs and unnatural proteins using the cation-depleted flexizyme-charged tRNAs in in vitro translation systems (see, FIG. 18A), and translation of complete or partial unnatural proteins, data storage, and ribosome/mRNA display using the cation-depleted flexizyme-charged tRNAs in in vitro translation systems (see, FIG. 18B).

In this experiment, unnatural amino acids are first charge onto unmodified tRNAs. The unnatural amino acids may include but not limited to D-amino acids and 3-amino acids, such as D-Phe, D-His, D-Cys, D-Ala, D-Ser, D-Met, D-Thr, D-Tyr, N-chloroacetyl-D-Tyr, D-Trp, N-chloroacetyl-D-Trp, L-O-homomethionine (P-hMet), L-O-homoglutamine (0-hGln), L-O-homophenylglycine (0-hPhg), 2-aminocyclohexanecarboxylic acid (2-ACHC) or 2-aminocyclopentanecarboxylic acid (2-ACPC). The flexizyme-charged tRNAs are purified by a technique including but not limited to HPLC to reduce cation contamination. Other purification techniques may include ultrafiltration and dialysis. The flexizyme-charged tRNAs are then concentrated to 100 to 500 μM and used as substrates for in vitro translation. The translation products are analyzed by MALDI-TOF MS and Tricine-SDS-PAGE.

As a proof-of-concept, a peptide drug is translated using the aaRS-free translation system (see, FIG. 18A).

The amino acid sequence of the peptide drug is AcyFAYDRR(2-ACHC)LSNN(2-ACHC)RNYcG-NH₂ (SEQ ID No. 124), where the first amino acid is an acetyl-D-Tyr, the penultimate amino acid is a D-Cys, which spontaneously forms a cyclic bond with the acetyl-D-Tyr residue. This peptide was previously shown to inhibit human factor XIIa. The translation products is analyzed by MALDI-TOF MS.

As another proof-of-concept, a protein enzyme, such as the 169-aa Gaussia luciferase, is translated using the cation-depleted flexizyme-charged tRNAs (see, FIG. 18A), including but not limited to tRNA^{Asn}, tRNA^{Ile}, and tRNA^{LYs}. The other tRNAs will be charged by recombinant aaRS proteins. In addition, an unnatural amino acid, the fluorescein labeled phenylalanine (Fph), is charged onto the initiator tRNA^{Met} by flexizyme. The translation products is analyzed by measuring bioluminescence, as well as SDS-PAGE. Because the Fph residue will make Gaussia luciferase fluoresce on the SDS-PAGE gel, the purity of the translated Gaussia luciferase can therefore be readily determined based on its fluorescence. This application is useful for high-throughput analysis of translation purity without the need for radioisotope and the laborious protein purification procedures.

The translation of complete or partial unnatural peptides using cation-depleted flexizyme-charged tRNAs used in conjunction with or without other aaRS proteins may find applications in the selection of peptide drugs, in conjunction with selection schemes such as ribosome display and mRNA display, as well as data storage through complete or partial unnatural peptides with amino acid letters (see, FIG. 18B).

### Example 12

### Charging of Enzymatically Transcribed L-tRNA Using L-Flexizyme

FIGS. 19A-B present 8% acid PAGE photographs and analysis of the experimental proof-of-concept of charging fully functional enzymatically transcribed L-tRNA molecules with pre-activated amino-acids, wherein FIG. 19A shows the results charging enzymatically transcribed L-tRNA and FIG. 19B shows the results charging synthetically generated L-tRNA.

As can be seen in FIGS. 19A-B, a band shift is revealed as the enzymatically transcribed L-tRNA becomes charged (FIG. 19A), whereas in the case of L-flexizyme charging of a pre-activated amino acid onto L-tRNA prepared by a commercial synthesizer, no band shift was observed and the charged L-tRNA molecules cannot be distinguished from the uncharged tRNA molecules, presumably due to poor quality of the synthetically prepared tRNAs.

This experiment clearly proves the benefits of obtaining enzymatically transcribed L-tRNA molecules, and that also show clearly the benefits of using L-flexizyme and obtaining D-enzymes that can enzymatically transcribe L-RNA molecules.

### Example 13

### Translation of Peptides Including Two or Three Consecutive D-Phenylalanine

To validate the effect of increasing the concentrations of cation-depleted tRNAs and whether it could improve the translation yield of challenging unnatural amino acids such as D-amino acids, the inventors attempted to translate a short peptide (mRNA #7): Fph-KKK^{D}FDFDYKDDDDK (SEQ ID No. 127), of which fluorescein-labeled L-phenylalanine (Fph) and L-lysine (K) were charged onto their cognate tRNAs by flexizyme, whereas L-aspartic acid (D) and L-tyrosine (Y) were charged onto their cognate tRNAs by aaRS (AspRS and TryRS, respectively).

Table 9 below presents tRNA sequences for in vitro translation of mRNA #7 to mRNA #10.

TABLE 9

| tRNA | Anticodon | Sequence | SEQ ID No. |
|---|---|---|---|
| Asp | GUC | 5′- | 113 |
| | | ggagcgguaguucagucgguuagaauaccugccugu | |
| | | cacgcagggggucgcggguucgagucccguccguuc | |
| | | cgcca-3′ | |
| fMet | CAU | 5′- | 114 |
| | | gggggguggagcagccugguagcucgucgggcuca | |
| | | uaacccgaagaucgucgguucaaauccggcccccgca | |
| | | acca-3′ | |
| GluE2 | CUA | 5′- | 115 |
| | | guccccuucgucuagaggcccaggacaccgcccucua | |
| | | acggcgguaacagggguucgaauccccuaggggacg | |
| | | cca-3′ | |
| Lys | CUU | 5′- | 116 |
| | | gggucguuagcucaguugguagagcaguugacucu | |
| | | uaaucaauuggucgcagguucgaauccugcacgacc | |
| | | cacca-3′ | |
| Phe | GAA | 5′- | 117 |
| | | gcccggauagcucagucgguagagcaggggauugaa | |
| | | aauccccguguccuugguucgauuccgaguccgggc | |
| | | acca-3′ | |
| Tyr | GUA | 5′- | 118 |
| | | ggugggguucccgagcggccaaagggagcagacugu | |
| | | aaaucugccgucaucgacuucgaagguucgaauccu | |
| | | ucccccaccacca-3′ | |

D-phenylalanine (^{D}F) was charged by flexizyme onto an engineered tRNA, tRNA^{G}UE²cu_{A}(see, Table 9), with sequence optimized for D-amino acids incorporation (^{D} PhetRNA^{G}oE²cu_{A}), following the teaching of Katoh, T. et al. [“Consecutive Elongation of D-Amino Acids in Translation”, Cell Chemical Biology, 2017, 24, pp. 46-54]. This peptide contains two consecutive D-phenylalanine, which was previously shown to be difficult to translate with less than 15% yield compared with the peptide of same sequence but contained two consecutive L-phenylalanine [Achenbach, J. et al., “Outwitting EF-Tu and the ribosome: translation with D-amino acids”, NucleicAcids Research, 2015, 43, pp. 5687-5698]. The inventors designed and in vitro transcribed mRNA #7 that allowed Watson-Crick base pairing to the anticodon of flexizyme-charged tRNAs (see, Table 10).

Table 10 below presents DNA template sequences for in vitro translation of mRNA #7 to mRNA #10.

TABLE 10

| | | SEQ |
|---|---|---|
| DNA templates | Sequence | ID No. |
| DNA template for | 5′- | 119 |
| mRNA #7 | GGCGTAATACGACTCACTATAGGGT | |
| | TAACTTTAAGAAGGAGATATACCAA | |
| | TGAAGAAGAAGTTCTTCTTCGACTA | |
| | CAAGGACGACGACGACAAGTAAGCT | |
| | TCG-3′ | |
| DNA template for | 5′- | 120 |
| mRNA #8 | GGCGTAATACGACTCACTATAGGGT | |
| | TAACTTTAAGAAGGAGATATACCAA | |
| | TGAAGAAGAAGTAGTAGGACTACAA | |
| | GGACGACGACGACAAGTAAGCTTC | |
| | G-3′ | |
| DNA template for | 5′- | 121 |
| mRNA #9 | GGCGTAATACGACTCACTATAGGGT | |
| | TAACTTTAAGAAGGAGATATACCAA | |
| | TGAAGAAGAAGTAGTAGTAGGACTA | |
| | CAAGGACGACGACGACAAGTAAGCT | |
| | TCG-3′ | |
| DNA template for | 5′- | 122 |
| mRNA #10 | GGCGTAATACGACTCACTATAGGGT | |
| | TAACTTTAAGAAGGAGATATACCAA | |
| | TGAAGAAGAAGTAGTAGTAGGACTA | |
| | CAAGGACGACGACGACAAGTAAGCT | |
| | TCG-3′ | |

The inventors have added 20 μM or 200 μM cation-depleted ^{D}Phe-tRNA^{Gluhu E2}_{CUA} for in vitro translation. For both translation reactions, the overall Mg²⁺carryover by charged-tRNAs was controlled within the herein-proposed limits of Mg²⁺tolerance for in vitro translation systems (<100 mM Mg²⁺). The inventors also translated mRNA #8 (Fph-^{L}K^{L}K^{L}K-^{L}F^{L}F^{L}F-^{L}D^{L}Y^{L}K^{L}D^{L}D^{L}D^{L}D^{L}K (SEQ ID No. 129) (see, Table 10) in parallel using flexizyme-charged tRNA^{Phe} (^{L}Phe-tRNA^{Phe}) as a control.

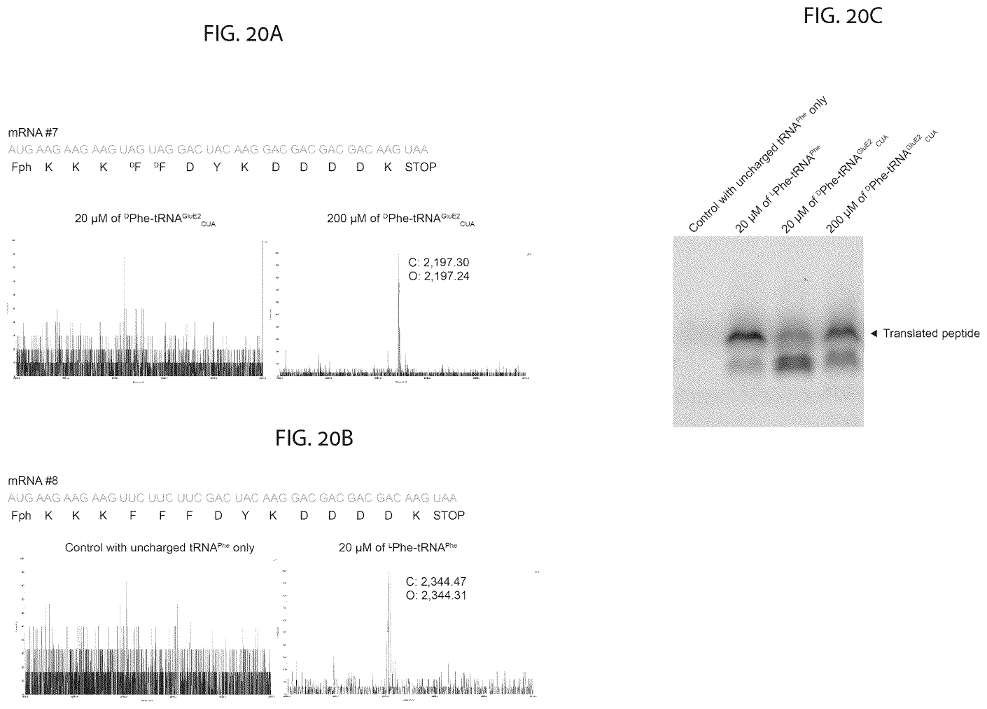

Translation reactions were incubated at 37° C. for 2 hours, and were analyzed by MALDI-TOF MS and 20% Tricine-SDS-PAGE. The MALDI-TOF MS results show accurate incorporation of two consecutive D-phenylalanine in mRNA #7 (FIG. 20A), but the mass peak could only be detected in samples with 200 μM ^{D}Phe-tRNA^{Gluhu E2}_{CUA} but not in those with 20 μM ^{D}Phe-tRNA^{Gluhu E2}_{CUA} (FIG. 20A), whereas in the control experiments, an accurate mass peak could be detected in samples with 20 μM ^{L}Phe-tRNA^{Phe} but not in those with uncharged tRNA^{Phe} only. Furthermore, the Tricine-SDS-PAGE results show that the translation yield of mRNA #7 with 200 μM ^{D}Phe-tRNA^{GluE2}_{CUA} was about 2-fold higher than that with 20 μM ^{D}Phe-tRNA^{Gluhu E2}_{CUA} and was similar to the control with 20 μM ^{L}Phe-tRNA^{Phe}

FIGS. 20A-C present the result of the in vitro translation of a short peptide containing two consecutive D-phenylalanine, wherein FIG. 20A shows MALDI-TOF-MS analysis of translated short peptides from mRNA #7, FIG. 20B shows MALDI-TOF-MS analysis of translated short peptides from mRNA #8, and FIG. 20C shows Tricine-SDS-PAGE analysis of translation products of mRNA #7 or mRNA #8 with uncharged tRNAPhe only (mRNA #7), 20 μM LPhe-tRNAPhe (mRNA #7), 20 μM Dphe-tRNAGluE2CUA (mRNA #8), or 200 μM Dphe-tRNAGluE2_{CUA} (mRNA #8), scanned by Typhoon FLA 9500 under Cy2 mode.

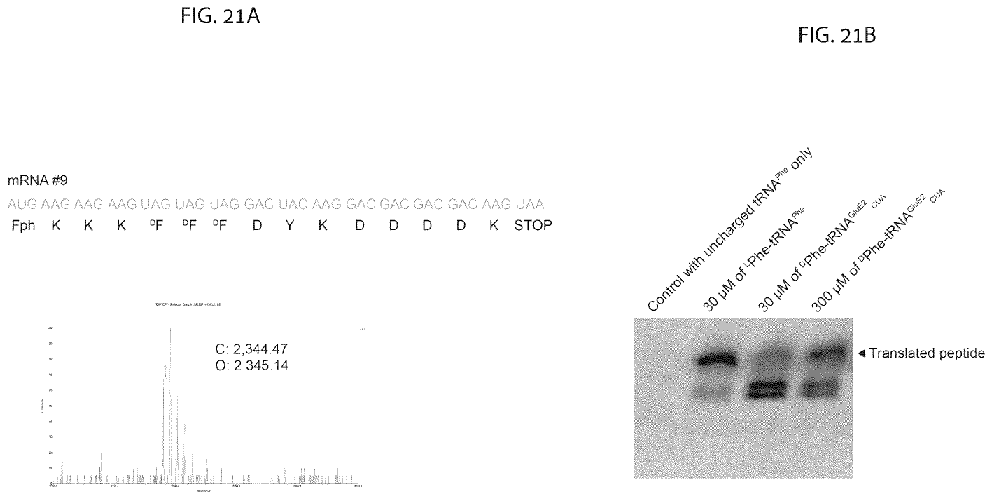

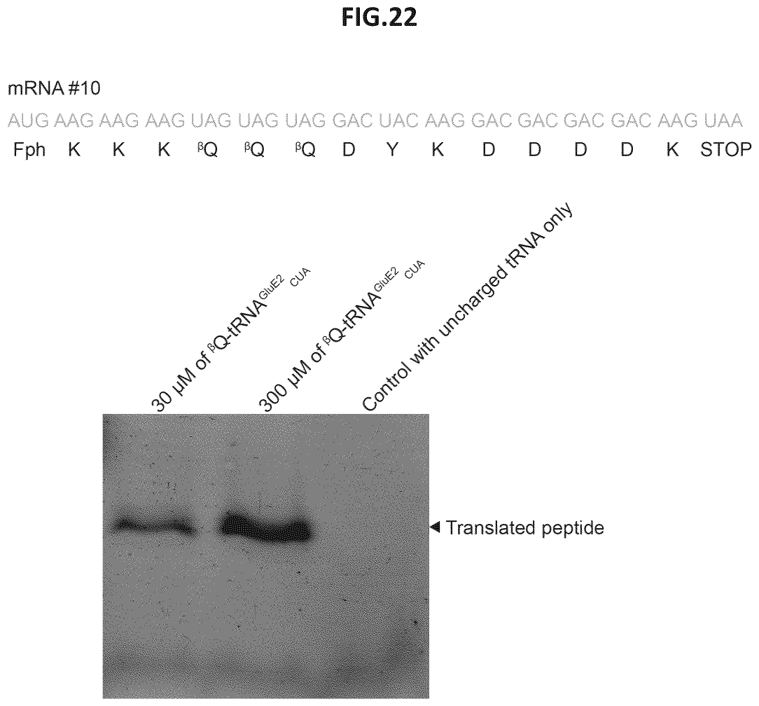

Encouraged by the successful translation of mRNA #7 with two consecutive D-phenylalanine, the inventors translated mRNA #9 into a short peptide Fph-KKK^{D}F^{D}F^{D}FDYKDDDDK (SEQ ID No. 127) with three consecutive D-phenylalanine (see, Table 10). Previous attempts to translate short a peptide with three consecutive D-phenylalanine showed less than 5% yield compared with the peptide of same sequence but contained three consecutive L-phenylalanine [Achenbach, J. et al., 2015]. The inventors added 30 μM or 300 μM cation-depleted ^{D}Phe-tRNAGluE2_{CUA} for in vitro translation, and analyzed the translation reaction by MALDI-TOF MS and 20% Tricine-SDS-PAGE. For both translation reactions, the overall Mg2+carryover by charged-tRNAs was controlled within the herein-proposed limits of Mg²⁺tolerance for in vitro translation systems (<100 mM Mg²⁺). The MALDI-TOF MS results show accurate incorporation of three consecutive D-phenylalanine in mRNA #9 (FIG. 21A). Furthermore, the Tricine-SDS-PAGE results show that the translation yield of mRNA #9 with 300 μM ^{D}Phe-tRNAGluE2CUA was about 2-fold higher than that with 30 μM ^{D}Phe-tRNAGluE2cUA and was similar to the control with 30 μM ^{L}Phe-tRNAPhe (FIG. 21B).

FIGS. 21A-B present the result of the in vitro translation of a short peptide containing three consecutive D-phenylalanine, wherein FIG. 21A shows MALDI-TOF-MS analysis of translated short peptides from mRNA #9, and FIG. 21B shows Tricine-SDS-PAGE analysis of translation products of mRNA #9 with uncharged tRNAPhe only, 30 μM LPhe-tRNAPhe, 30 μM ^{D}Phe-tRNAGluE2_{CUA}, or 300 μM ^{D}Phe-tRNAGluE2_{CUA}, scanned by Typhoon FLA 9500 under Cy2 mode.

Taken together, these results suggest that by increasing the concentrations of cation-depleted flexizyme-charged tRNAs from about 20-30 μM to about 200-300 μM, the incorporation efficiencies of D-amino acids (up to three consecutive D-phenylalanine) was significantly improved.

### Example 14

### Translation of Peptides Including Three Consecutive β-Amino Acids

To test if increasing the concentrations of cation-depleted tRNAs could improve the translation yield of β-amino acids, the inventors have attempted to translate a short peptide (mRNA #10): Fph-KKK^{βl Q}^{βl Q}^{βl QDYKDDDDK (SEQ ID No.} 129) (see, Table 10). The inventors added 30 μM or 300 μM cation-depleted ^{β}Q-tRNAGluE2_{CUA} for in vitro translation, and analyzed the translation reaction by 20% Tricine-SDS-PAGE.

In both translation reactions, the overall Mg²⁺carryover by charged-tRNAs was controlled within the herein-proposed limits of Mg²⁺tolerance for in vitro translation systems (less than 100 mM Mg²⁺). The translation products were purified by ANTI-FLAG M2 magnetic beads (Sigma), so that full-length translation products were separated from those truncated translation products.

The Tricine-SDS-PAGE results show that the translation yield of mRNA #10 with 300 μM βGln-tRNAGluE2CUA was slightly higher than that with 30 μM ^{β}Gln-tRNAGluE2CUA (FIG. 22).

FIG. 22 presents the results of the in vitro translation of a short peptide containing three consecutive β-Gln, showing the Tricine-SDS-PAGE analysis of translation products of mRNA #10 with uncharged tRNA only, 30 μM βGln-tRNAGluE2_{CUA}, or 300 μM βGln-tRNAGluE2CUA, scanned by Typhoon FLA 9500 under Cy2 mode.

Taken together, these results suggest that by increasing the concentrations of cation-depleted flexizyme-charged tRNAs from about 30 μM to about 300 μM, the incorporation efficiencies of B-amino acids (up to three consecutive B-Gln) was improved.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A system for producing a protein, comprising:
an mRNA molecule encoding the protein;
a plurality of charged tRNA molecules; and
a cell-free translation mix,
wherein a concentration of Mg²⁺, in the system is less than 100 mM.

2. The system of claim 1, essentially devoid of an aminoacyl tRNA synthetase (aaRS).

3-4. (canceled)

5. The system of claim 1, wherein at least one tRNA molecule of said plurality of charged tRNA molecules is charged by a ribozyme.

6. The system of claim 5, wherein said tRNA molecule is charged with an unnatural amino acid residue, wherein said unnatural amino acid residue is a D-amino acid residue.

7. The system of claim 6, wherein said unnatural amino acid residue is a D-amino acid residue.

8. The system of claim 7, wherein said tRNA molecule comprises L-ribonucleic acid residues (L-tRNA).

9. The system of claim 8, wherein said L-tRNA is prepared using a D-polymerase.

10. The system of claim 9, wherein said D-polymerase is a mirror-image protein of Dpo4 (D-Dpo4).

11. (canceled)

12. The system of claim 7, wherein said ribozyme comprises L-ribonucleic acid residues.

13. (canceled)

14. A method of producing a protein using the system of claim 1, comprising:
providing said plurality of charged tRNA molecules having no more than said concentration of Mg²⁺; and
contacting said plurality of charged tRNA molecules with said mRNA molecule encoding the protein in said cell-free translation mix, to thereby obtain the protein.

15. The method of claim 14, wherein said providing comprises, prior to said contacting, adjusting said concentration of Mg²⁺.

16. (canceled)

17. The method of claim 14, wherein said providing further comprises adjusting a concentration of said charged tRNA molecules to greater than 2-fold of a concentration of a charged tRNA in protein translation systems that include aaRS.

18. (canceled)

19. A method of charging an L-tRNA with a D-amino acid, comprising:
preparing the L-tRNA molecule using a D-polymerase;
providing an activated D-amino acid;
providing an L-aminoacylation ribozyme; and
contacting said L-tRNA, said L-aminoacylation ribozyme and said activated D-amino acid to thereby obtain a D-amino acid-charged L-tRNA molecule.

20. (canceled)

21. The method of claim 19, wherein said L-aminoacylation ribozyme is an L-flexizyme.

22. An L-flexizyme comprising of L-ribonucleotide residues.

23. The L-flexizyme of claim 22, comprising at least 50% L-ribonucleotide residues.

24-25. (canceled)

26. A protein prepared by the method of claim 14.

27. The protein of claim 26, selected from the group consisting of a protein that comprises at least one non-canonical amino acid residue, a protein that comprises at least one D-amino acid residue, an L-protein and a D-protein.

28. The protein of claim 27, selected from the group consisting of chicken lysozyme, Gaussia luciferase, and E. coli TrpRS.

29. (canceled)

30. The protein of claim 26, encoded by mRNA #6.

31. (canceled)
